(19) United States
(12) Patent Application Publication
(10) Pub. No.: US 2019/0083420 A1
(43) Pub. Date: Mar. 21, 2019

(54) METHODS OF USING (2R, 6R)-HYDROXYNORKETAMINE AND (2S, 6S)-HYDROXYNORKETAMINE IN THE TREATMENT OF DEPRESSION, ANXIETY, ANHEDONIA, FATIGUE, SUICIDAL IDEATION, AND POST TRAUMATIC STRESS DISORDERS

(71) Applicants: The Uinited States of America, as represented by the Secretary, Department of Health and Human Serv, Bethesda, MD (US); University of Maryland, Baltimore, MD (US)

(72) Inventors: Irving Wainer, Washington, DC (US); Carlos Zarate, Germantown, MD (US); Ruin Moaddel, Bel Air, MD (US); Todd Gould, Elkridge, MD (US); Panos Zanos, Baltimore, MD (US); Craig Thomas, Gaithersburg, MD (US); Patrick Morris, Columbia, MD (US)

(21) Appl. No.: 16/088,294

(22) PCT Filed: Mar. 27, 2017
(86) PCT No.: PCT/US2017/024238

§ 371 Date: Sep. 25, 2018

Related U.S. Application Data
(60) Provisional application No. 62/313,317, filed on Mar. 25, 2016.

(51) Int. Cl. A61K 31/135; A61P 25/24

(52) U.S. Cl. CPC A61K 31/135; A61P 25/24

(57) ABSTRACT

Disclosed is a method of treating Psychotic Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, Unspecified Anxiety Disorder, or fatigue the method including administering a pharmaceutical composition containing an effective amount of an active agent, wherein the active agent is purified (2R,6R)-hydroxynorketamine, purified (2S,6S)-hydroxynorketamine, or a combination thereof, or a pharmaceutically acceptable salt thereof, together with a pharmaceutically acceptable carrier to a patient in need of such treatment.

### CROSS-REFERENCE TO RELATED APPLICATION

This application claims priority to U.S. Provisional Patent Application No. 62/313,317, filed on Mar. 25, 2016, in the United States Patent and Trademark Office, and all the benefits accruing therefrom under 35 U.S.C. § 119, the content of which is incorporated herein in its entirety by reference.

### STATEMENT OF GOVERNMENT SUPPORT

This invention was made with government support under Grant Number NH099345 awarded by the National Institutes of Health. The United States government has certain rights in the invention.

### BACKGROUND

Ketamine, a drug currently used in human anesthesia and veterinary medicine, has been shown in clinical studies to be effective in the treatment of several conditions, including treatment-resistant bipolar depression, major depressive disorder, anhedonia, fatigue, and suicidal ideation.

However, ketamine is only approved for use as an anesthetic. Use of the drug for other indications is hindered by unwanted central nervous system (CNS) effects. Approximately 30% of patient population does not respond to ketamine treatment. Additionally, ketamine treatment is associated with serious side effects due to the drug's anesthetic properties and abuse potential. The mechanism of action for ketamine in depression is not known, which provides uncertainty as to whether it would be possible to generate ketamine analogs which retain antidepressant activity but avoid undesired side effects.

Ketamine analogs have potential advantages over standard antidepressants, as the time to efficacy of ketamine is rapid and takes effect within hours or minutes, unlike selective serotonin reuptake inhibitors (SSRIs) and other standard of care antidepressants from different chemical classes (e.g., serotonin and norepinephrine reuptake inhibitors (SNRIs), monoamine oxidase inhibitors, tricyclic antidepressants, noradrenergic and specific serotonergic antidepressants which require several weeks to have an effect. Further, there are patients who respond to the antidepressant effects of ketamine but do not respond to SSRIs or other antidepressants.

Thus, the need for therapeutics which exhibit the therapeutic properties of ketamine with efficacy in a higher percentage of patients, reduced anesthetic properties and reduced abuse liability exists. The present disclosure fulfills this need and provides additional advantages set forth herein.

### FIELD OF THE DISCLOSURE

This disclosure demonstrates that (2R,6R)-hydroxynorketamine (2R,6R-HNK) and (2S,6S)-hydroxynorketamine (2S,6S-HNK) can be used in the treatment of CNS disorders and conditions, including depression, anxiety, anhedonia, fatigue, suicidal ideation, and post traumatic stress disorders. The disclosure provides methods of treatment including use of pharmaceutical preparations containing the above mentioned compounds. The disclosure provides methods of treating various CNS disorders by administering purified (2R,6R)-HNK or (2S,6S)-HNK to patients in need of such treatment.

### SUMMARY

In a first aspect the disclosure provides a method of treating Psychotic Depression, Major Depressive Disorder, Bipolar Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, Anhedonia, Post Traumatic Stress Disorder, Unspecified Anxiety Disorder, or fatigue, including fatigue related to mental or medication conditions (e.g, Chronic Fatigue Syndrome, fatigue associated with cancer or other medical conditions or medications to treatment these disorders or conditions), and equivalent disorders or conditions as specified by the DSM 5, IC-10, and IC-11, and maladaptive functions of RDoc domains such as negative valence systems, positive valence systems, cognitive systems, systems for social processes, and arousal/regulatory systems, the method including administering a pharmaceutical composition containing an effective amount of an active agent, wherein the active agent is purified (2R,6R)-hydroxynorketamine, purified (2S,6S)-hydroxynorketamine, a prodrug thereof, or a pharmaceutically acceptable salt of any of the foregoing, or a combination of any of the foregoing, together with a pharmaceutically acceptable carrier, which may include modifiers including buffers, tonicity adjusters and stability adjusters, to a patient in need of such treatment.

### BRIEF DESCRIPTION OF DRAWINGS

FIG. 1. The role of NMDA receptor and metabolism in the antidepressant actions of ketamine Graphs of immobility time (sec) versus dose (mg/kg) for 1a, (R,S)-ketamine (KET), desipramine and 1b, MK-801 in the forced-swim test 1- and 24-hours post-treatment. 1c, Graphs of Graph of latency to feed (sec) versus dose (mg/kg) for novelty-suppressed feeding. 1d, Graph of escape failures versus dose (mg/kg) for learned helplessness paradigms 1e, Graph of immobility time (sec) versus dose (mg/kg) for MK-801 and R,S-ketamine (racemic). 1f, Simplified diagram of (R,S)-KET's metabolism. 1g, Graph of immobility time (sec) vs dose for (mg/kg) showing effects of (R,S)-KET and d-(R,S)-KET in the forced-swim test 1- and 24-hours post-administration. Graphs of drug brain levels (μg/kg) versus time post-injection (min) for 1h, KET, nor-KET and 1j, (2S,6S;2R,6R)-hydroxynorketamine (HNK) following administration. In this and all following figures *'s indicate data are means±S.E.M. *p<0.05, **p<0.01, ***p<0.001.

FIG. 2. The antidepressant actions of ketamine's metabolite (2R,6R)-HNK are mediated via a non-NMDA receptor-dependent mechanism. (2a-2c), Brain levels of 2a, KET, 2b, nor-KET and 2c, (2S,6S;2R,6R)-hydroxynorketamine (HNK) following administration of (R,S)-KET and 6,6-dideuteroketamine ((R,S)-d2-KET). (2d-2e), Effects of (R,S)-KET and (R,S)-d2-KET in the 2d, 1- and 24-hours forced-swim test and the 2e, learned helplessness test. (2f-2g), Compared to (2S,6S)-HNK, (2R,6R)-HNK manifested greater potency and longer-lasting antidepressant-like effects in the 2f, forced-swim test and 2g, learned helplessness paradigms 2h, (2R,6R)-HNK reversed social interaction deficits induced by chronic social defeat stress.

FIG. 3. Activation of AMPA receptors is necessary for the antidepressant effects of (2R,6R)-HNK. 3a, Representative spectrograms for 10-min prior (baseline) and 1-hour after administration of (R,S)-ketamine or (2R,6R)-HNK (indicated by a dashed line). 3b, Normalized gamma power changes following administration of (R,S)-KET, (2R,6R)-HNK, or vehicle (3c, 3d). Pre-treatment with the AMPA receptor inhibitor NBQX 10 minutes prior to (R,S)-ketamine (KET) and (2R,6R)-hydroxynorketamine (HNK) prevented their antidepressant-like actions in the 3d, 1-hour or 3d, 24-hours forced-swim test. (3e-3f) Effects of (R,S)-KET and (2R,6R)-HNK on levels of GluR1 and GluR2 proteins in synaptoneurosomes of hippocampus 3e, 1-hour and 3f, 24-hours post-treatment.

FIG. 4. (2R,6R)-HNK lacks ketamine-related side effects. (4a, 4b), After recording baseline activity for 1 hour, mice received drug (marked by a vertical dashed line) and locomotor activity was monitored for another 1 hour. 4a, Administration of (2S,6S)-hydroxynorketamine (HNK) dose-dependently changed locomotor activity, while administration of 4b, (2R,6R)-HNK did not. 4c, (2S,6R)-HNK, but not 4d, (2R,6R)-HNK, induced motor incoordination in the rotarod paradigm. Unlike (R,S)-KET, (2R,6R)-HNK administration did not induce 4e, pre-pulse inhibition deficits, (4f, 4g), (R,S)-KET-associated discriminative stimulus. Data are means±S.E.M. *p<0.05, **, p<0.01, ***p<0.001, KET vs saline (SAL); for panel 4c, * (R,S)-KET, # (2S,6S)-HNK.

FIG. 5. Ketamine's in vivo metabolic transformations. Ketamine is metabolised in vivo via P450 enzymatic transformations. (i) (R,S)-Ketamine (KET) is selectively demethylated to give (R,S)-norketamine (norKET). (ii) NorKET can be then dehydrogenated to give (R,S)-dehydronorketamine (DHNK). (iii) Alternatively, norKET can be hydroxylated to give the hydroxynorketamines (HNKs). (iv) (R,S)-KET can also be hydroxylated at the 6-position to give either the E-6-hydroxyketamine ((2S,6R;2R,6S)-HK)) or Z-6-hydroxyketamine ((2S,6S;2R,6R)-HK)). (v) Demethylation of (2S,6R;2R,6S)-HK yields the production of (2S,6R;2R,6S)-hydroxynorketamine (HNK). (vi) Demethylation of (2S,6S;2R,6R)-HK further gives (2S,6S;2R,6R)-hydroxynorketamine (HNK).

FIG. 6. Circulating levels of ketamine and its metabolites following i.p. administration in mice. 6a, Plasma and 6b, brain levels of ketamine (KET) and its metabolites following administration of (R,S)-KET (10 mg/kg) in mice. (6c-6e) Brain levels of 6c, KET, 6d, norketamine (norKET) and 6e, hydroxynorketamine (HNK) following administration of (S)- and (R)-KET. 6f, Chemical structure of (R,S)-6,6-dideuteroketamine ((R,S)-d2-KET).

FIG. 7. Extended Data FIG. 3. Ketamine, but not MK-801, reverses social defeat stress-induced social avoidance. 7a, Chronic social defeat stress and social interaction/avoidance test timeline. (7b-7c), A single injection of (R,S)-ketamine (KET), but not MK-801, reversed social defeat stress-induced social avoidance behaviors in mice, without affecting 7d, locomotor activity or e, total number of compartmental crosses in the social interaction apparatus. Data are means±S.E.M. ***p<0.001. SAL, saline.

FIG. 8. Locomotor effects of (R,S)-ketamine, (R,S)-6,6-dideuteroketamine, (2S,6S)-hydroxynorketamine and (2R,6R)-hydroxynorketamine in the open-field test. After recording baseline activity for 60 min, animals received drug (marked by a vertical dashed line) and locomotor activity was monitored for another 1 hour. (8a, 8b), (R,S)-ketamine (KET) and (R,S)-6,6-dideuteroketamine ((R,S)-d₂-KET) were equally potent in inducing a hyper-locomotor response at the dose of 10 mg/kg. (8c, 8d), Administration of (R,S)-KET (10 mg/kg), induced hyper-locomotor responses equally in both male and female mice. Data are means±S.E.M. *p<0.05, **p<0.01. SAL, saline.

FIG. 9. Acute and long-lasting antidepressant-like and anti-anhedonic effects of (2R,6R)-hydroxynorketamine 9a, A single injection of (2S,6S)-hydroxynorketamine (HNK) induced antidepressant-like effects in the learned helplessness at the dose of 75 mg/kg. 9b, A single injection of (2R,6R)-HNK resulted in dose-dependent antidepressant-like responses at the doses of 5-75 mg/kg. 9c, Despite the greater antidepressant efficacy of (2R,6R)-HNK, administration of (2S,6S)-HNK (HNK) results in higher brain hydroxynorketamine levels compared to (2R,6R)-HNK. (9d-9e), (2R,6R)-HNK manifested dose-dependent antidepressant-like effects in the 9d, novelty-suppressed feeding and 9e, forced-swim test 1- and 24-hours post-injection. 9f, Similar to (R,S)-ketamine (KET), the antidepressant-like effects of (2R,6R)-HNK persisted for at least 3 days post-treatment. 9g, A single administration of (2R,6R)-HNK reversed chronic corticosterone-induced decreases in sucrose preference. 9h, A single administration of (2R,6R)-HNK reversed chronic corticosterone-induced decrease in female urine sniffing preference, specifically in mice that developed an anhedonic phenotype. (9i-9j) Administration of (2R,6R)-HNK was not associated with changes in 9i, locomotor activity or 9j, total compartmental crosses in the social interaction test following chronic social defeat stress; SAL, saline.

FIG. 10. Administration of the AMPA receptor inhibitor NBQX, 30 min prior to the 24-hour forced-swim test prevented the antidepressant effects of both (R,S)-KET and (2R,6R)-HNK. Data are means±S.E.M. *p<0.05, **p<0.01, ***p<0.001. Abbreviations: NBQX, 2,3-dihydroxy-6-nitro-7-sulfamoyl-benzo[f]quinoxaline-2,3-dione; SAL, saline; SLM, stratum lacunosum-moleculare; SO, stratum oriens; SP, stratum pyramidale; SR, stratum radiatum.

FIG. 11. Administration of the AMPA receptor antagonist, NBQX, prevents (2R,6R)-HNK-induced increases in gamma oscillations in vivo. 11a, Administration of (R,S)-ketamine (KET), but not (2R,6R)-hydroxynorketamine (HNK), increased locomotor home-cage activity of mice. Neither (R,S)-KET, nor (2R,6R)-HNK altered cortical 11b, alpha, 11c, beta, 11d, delta or 11e, theta oscillations in vivo. (11f-11k) Pre-treatment with the AMPA receptor antagonist, NBQX, did not change the 11f, locomotor activity, 11g, alpha, 11h, beta, 11j, delta or 11k, theta oscillations, but it 11i, prevented (2R,6R)-HNK-induced increases of gamma oscillations in vivo. Data are means±S.E.M. NBQX, 2,3-dihydroxy-6-nitro-7-sulfamoyl-benzo[f]quinoxaline-2,3-dione; SAL, saline.

FIG. 12. Effects of (2R,6R)-hydroxynorketamine on synaptoneurosome protein and protein phosphorylation levels. A single administration of (R,S)-ketamine (KET, 10 mg/kg) or (2R,6R)-hydroxynorketamine (HNK, 10 mg/kg) (12a, 12b), did not alter synaptoneurosome levels of mTOR or phosphorylated mTOR 1- or 24-hours post-injection (12c, 12d), but it did decrease phosphorylation of eEF2, 1-hour and 24 hours post-injection, and (12e, 12f), increased mBDNF levels 24 hours post-administration in the hippocampus of mice. Administration of (R,S)-KET or (2R,6R)-HNK (12g, 12h), did not alter synaptoneurosome levels of GluR1/GluR2, (12i, 12j), mTOR/phosphorylated mTOR, (12k, 12l), eEF2/phosphorylated eEF2 or (12m, 12n), proBDNF/mBDNF in the prefrontal cortex of mice. The values for the phosphorylated forms of proteins were normalized to phosphorylation-independent levels of the same protein. Phosphorylation-independent levels of proteins were normalized to GAPDH. Data are means±S.E.M, and was normalized to the saline-treated control group for each protein. *p<0.05. Abbreviations: eEF2, eukaryotic translation elongation factor 2; GAPDH, Glyceraldehyde 3-phosphate dehydrogenase; mBDNF, mature brain-derived neurotrophic factor; mTOR, mammalian target of rapamycin; proBDNF, pro-brain-derived neurotrophic factor; SAL, saline.

FIG. 13. Effects of (2R,6R)-hydroxynorketamine administration on startle amplitude and drug discrimination response rate. 13a, Startle amplitude in the pre-pulse inhibition task was not affected by administration of (R,S)-ketamine (KET) or (2R,6R)-hydroxynorketamine (HNK). (13b, 13c), Response rate of overall lever pressing per sec in the drug discrimination paradigm was not changed by administration of 13b, (R,S)-KET, (2R,6R)-HNK or 13c, phencyclidine (PCP).

FIG. 14. Single crystal X-ray structure of (2S,6S)-(+)-hydroxynorketamine hydrochloride.

FIG. 15. Single crystal X-ray structure of (2R,6R)-(−)-hydroxynorketamine hydrochloride.

### DETAILED DESCRIPTION

### Terminology

Compounds disclosed herein are described using standard nomenclature. Unless defined otherwise, all technical and scientific terms used herein have the same meaning as is commonly understood by one of skill in the art to which this disclosure belongs.

The terms “a” and “an” do not denote a limitation of quantity, but rather denote the presence of at least one of the referenced item.

The term “chiral” refers to molecules, which have the property of non-superimposability of the mirror image partner.

“Stereoisomers” are compounds, which have identical chemical constitution, but differ with regard to the arrangement of the atoms or groups in space.

A “Diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization or chromatography, using, for example via HPLC.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

Where a compound exists in various tautomeric forms, the invention is not limited to any one of the specific tautomers, but rather includes all tautomeric forms.

The disclosure includes compounds having all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

An “active agent” means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms.

“Depressive symptoms” include low mood, diminished interest in activities, psychomotor slowing or agitation, changes in appetite, poor concentration or indecisiveness, or other cognitive symptoms associated with depression, excessive guilt or feelings of worthlessness, low energy or fatigue, and suicidal ideations may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders, personality disorders, and symptoms such as anhedonia. The longitudinal course of the disorder, the history and type of symptoms, and etiologic factors help distinguish the various forms of mood disorders from each other.

“Depression symptom rating scale” refers to any one of a number of standardized questionnaires, clinical instruments, or symptom inventories utilized to measure symptoms and symptom severity in depression. Such rating scales are often used in clinical studies to define treatment outcomes, based on changes from the study's entry point(s) to endpoint(s). Such depression symptoms rating scales include, but are not limited to, The Quick Inventory of Depressive-Symptomatology Self-Report (QIDS-SR₁₆), the Beck Depression Inventory (BDI), the 17-Item Hamilton Rating Scale of Depression (HRSD₁₇), the 30-Item Inventory of Depressive Symptomatology (IDS-C₃₀), or The Montgomery-Asperg Depression Rating Scale (MADRS). Such ratings scales may involve patient self-report or be clinician rated. A 50% or greater reduction in a depression ratings scale score over the course of a clinical trial (starting point to endpoint) is typically considered a favorable response for most depression symptoms rating scales. “Remission” in clinical studies of depression often refers to achieving at, or below, a particular numerical rating score on a depression symptoms rating scale (for instance, less than or equal to 7 on the HRSD₁₇; or less than or equal to 5 on the QIDS-SR₁₆; or less than or equal to 10 on the MADRS).

“Anxiety symptom rating scale” refers to any one of a number of standardized questionnaires, clinical instruments, or symptom inventories utilized to measure symptoms and symptom severity in anxiety. Such rating scales are often used in clinical studies to define treatment outcomes, based on changes from the study's entry point(s) to endpoint(s). Such anxiety symptoms rating scales include, but are not limited to, State-Trait Anxiety Inventory (STAI), the Hamilton Anxiety Rating Scale (HAM-A), the Beck Anxiety Inventory (BAT), and the Hospital Anxiety and Depression Scale-Anxiety (HADS-A). Such ratings scales may involve patient self-report or be clinician rated. A 50% or greater reduction in a depression or anxiety ratings scale score over the course of a clinical trial (starting point to endpoint) is typically considered a favorable response for most depression and anxiety symptoms rating scales. “Remission” in clinical studies of depression often refers to achieving at, or below, a particular numerical rating score on a depression symptoms rating scale (for instance, less than or equal to 39 on the STAI; or less than or equal to 9 on the BAI; or less than or equal to 7 on the HADS-A).

“Anhedonia rating scale” refers to any one of a number of standardized questionnaires, clinical instruments, or symptom inventories utilized to measure severity of anhedonia. Such anhedonia symptoms rating scales include, but are not limited to, Shaith-Hamilton Pleasure Scale (SHAPS and SHAPS-C) and the Temporal Experience of Pleasure Scale (TEPS).

“Fatigue rating scale” refers to any one of a number of standardized questionnaires, clinical instruments, or symptom inventories utilized to measure presence and severity of fatigue. Such fatigue symptoms rating scales include the 7 item NIH-Brief Fatigue Inventory (NIH-BFI), the 13 item Functional Assessment of Chronic Illness Therapy-Fatigue (FACIT-F), and the 7 item Patient Reported Outcomes Measurement Information System (PROMIS)—fatigue short form, and the 27 item multidimensional revised Piper Fatigue Scale (rPFS).

“Suicidal ideation rating scale” refers to any one of a number of standardized questionnaires, clinical instruments, or symptom inventories utilized to measure severity of suicide ideation. Such suicidal ideation symptoms rating scales include, but are not limited to, Scale for Suicidal Ideation (SSI), the Suicide Status Form (SSF), or the Columbia Suicide Severity Rating Scale (C-S SRS).

A “patient” means any human or non-human animal in need of medical treatment. Medical treatment can include treatment of an existing condition, such as a disease or disorder, prophylactic or preventative treatment in patients known to be at risk for experiencing symptoms of anxiety or depression, or diagnostic treatment. In some embodiments the patient is a human patient.

“Pharmaceutical compositions” are compositions comprising at least one active agent, such as a (2S,6S)-HNK, (2R,6R)-HNK, or a salt, hydrate, or prodrug thereof, and at least one other substance, such as a carrier.

The term “carrier” applied to pharmaceutical compositions of the invention refers to a diluent, excipient, or vehicle with which an active compound is administered.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use.

“Pharmaceutically acceptable salts” are derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ-COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

“Prodrug” means any compound that becomes compound of the invention when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of the invention.

The term “therapeutically effective amount” or “effective amount” means an amount effective, when administered to a human or non-human patient, to provide any therapeutic benefit. A therapeutic benefit may be an amelioration of symptoms, e.g., an amount effective to decrease the symptoms of a depressive disorder or pain. A therapeutically effective amount of a compound is also an amount sufficient to provide a significant positive effect on any indicia of a disease, disorder or condition, e.g., an amount sufficient to significantly reduce the frequency and severity of depressive symptoms or pain. A significant effect on an indicia of a disorder or condition includes a statistically significant in a standard parametric test of statistical significance such as Student's T-test, where p<0.05; though the effect need not be significant in some embodiments.

### Chemical Description

It is disclosed herein that a ketamine metabolite Z-6-hydroxynorketamine (2,6-HNK) is critical for ketamine's antidepressant, anxiolytic, anti-anhedonic, and other behavioral effects. (2R,6R)-2-amino-2-(2-chlorophenyl)-6-hydroxycyclohexanone ((2R,6R)-hydroxynorketamine (HNK)) exerts rapid and sustained antidepressant, anxiolytic, and anhedonic effects. This compound has the structure

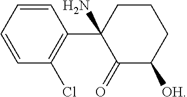 [SMILES: N[C@@]1(c2ccccc2Cl)CCC[C@@H](O)C1=O]

(2R,6R)-2-amino-2-(2-chlorophenyl)-6-hydroxycyclohexanone ((2R,6R)-hydroxynorketamine (HNK)) also exhibits antidepressant, anxiolytic, anti-anhedonic effects. This compound has the structure

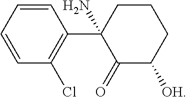 [SMILES: N[C@]1(c2ccccc2Cl)CCC[C@H](O)C1=O]

The terms “purified HNK,” “purified 2,6-HNK,” “purified 2R,6R-HNK,” and “ipurified 2S,6S-HNK” are used in the specification and claims to indicate that the HNK is administered rather than ketamine, which would then generate HNK by its metabolism. The activity of α-amino-3-hydroxy-5-methyl-4-isoxazole propionic acid (AMPA) receptors rather than the NMDA receptor inhibition is believed to be associated with this outcome. It is further shown that (2R,6R)-HNK lacks psychotomimetic effects, locomotor effects, discoordination, and addictive potential. Details of the experiments and results supporting these showings can be found in the Examples section.

### Prodrugs

2,6-HNK prodrugs are also useful in the methods of treatment disclosed herein. 2,6-HNK prodrugs include ester conjugates of the 6-hydroxy group of 2,6-HNK and amine conjugates of the 2,6-HNK amino group.

For example the disclosure includes the following prodrugs and their pharmaceutically acceptable salts.

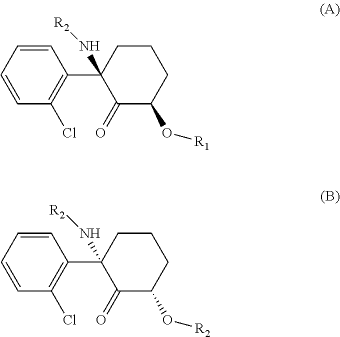

In prodrugs (A) and (B) the variables R₁ and R₂ carry the following definitions:

R₁ is hydrogen and R₂ is -A₂B₂ or R₁ is -A₁B₁ and R₂ is hydrogen.

-A₁B₁ is a group in which A₁ is —(C═O)—, —(C═O)O—, —(C═O)NHR, —(C═O)NRR, —S(O)₂, —S(O)₃, —P(O)₃, and B₁ is C₁-C₈alkyl, C₂-C₈alkenyl, C₂-C₈alkynyl, (carbocycle)C₀-C₄alkyl or (heterocycle)C₀-C₄alkyl, each of which is substituted with from 0 to 4 substituents independently chosen from halogen, hydroxyl, amino, cyano, C₁-C₄alkyl, C₁-C₄alkoxy, C₁-C₆alkylester, mono- and di-(C₁-C₄alkyl)amino, (C₃-C₇cycloalkyl)C₀-C₂alkyl, (heterocycloalkyl)C₀-C₂alkyl, C₁-C₂haloalkyl, and C₁-C₂haloalkoxy.

-A₂B₂ is a group in which A₂ is a bond, —(C═O)—, —(C═O)O—, —(C═O)NHR₆, —(C═O)NRR, —S(O)₂, —S(O)₃, —P(O)₃, B₂ is H, C₁-C₈alkyl, C₂-C₈alkenyl, C₂-C₈alkynyl, C₂-C₆alkanoyl, (carbocycle)C₀-C₄alkyl, (heterocycle)C₀-C₄alkyl, or an amino acid or dipeptide covalently bound to A₂ by its C-terminus, each of which is substituted with from 0 to 4 substituents independently chosen from halogen, hydroxyl, amino, cyano, C₁-C₄alkyl, C₁-C₄alkoxy, C₁-C₆alkylester, mono- and di-(C₁-C₄alkyl)amino, (C₃-C₇cycloalkyl)C₀-C₂alkyl, (heterocycloalkyl)C₀-C₂alkyl, C₁-C₂haloalkyl, and C₁-C₂haloalkoxy.

R is independently chosen at each occurrence from hydrogen and C₁-C₆alkyl.

In certain embodiments of prodrugs (A) and (B) have the definitions below.

(1) R₂ is -A₂B₂ where A₂ is a bond, —(C═O)O—, —S(O)₂-, —(S═O)NR—, or —(C═O)NR—, B₂ is C₁-C₆alkyl, C₂-C₄alkanoyl, (phenyl)C₀-C₂alkyl, (C₃-C₇cycloalkyl)C₀-C₄alkyl, (heterocycloalkyl)C₀-C₂alkyl, (5- or 6-membered heteroaryl)C₀-C₂alkyl, or an amino acid covalently bound to A₂ by its C-terminus, each of which is substituted with from 0 to 4 substituents independently chosen from halogen, hydroxyl, amino, cyano, C₁-C₄alkyl, C₁-C₄alkoxy, C₁-C₆alkylester, mono- and di-(C₁-C₄alkyl)amino, C₁-C₂haloalkyl, and C₁-C₂haloalkoxy.

(2) A₂ is a bond or —(C═O)O— and B₂ is C₂-C₆alkyl, (phenyl)C₀-C₂alkyl, or (C₃-C₇alkyl)C₀-C₄alkyl, each of which is substituted with from 0 to 4 substituents independently chosen from halogen, hydroxyl, amino, cyano, C₁-C₄alkyl, C₁-C₄alkoxy, and mono- and di-(C₁-C₄alkyl)amino.

(3) A₁ is —(C═O)— and B₁ is C₁-C₆alkyl, (phenyl)C₀-C₄alkyl, (C₃-C₇cycloalkyl)C₀-C₄alkyl, (heterocycloalkyl)C₀-C₂alkyl, or (5- or 6-membered heteroaryl)C₀-C₂alkyl, each of which is substituted with from 0 to 4 substituents independently chosen from halogen, hydroxyl, amino, cyano, C₁-C₄alkyl, C₁-C₄alkoxy, C₁-C₆alkylester, mono- and di-(C₁-C₄alkyl)amino, (C₃-C₇cycloalkyl)C₀-C₂alkyl, (heterocycloalkyl)C₀-C₂alkyl, C₁-C₂haloalkyl, and C₁-C₂haloalkoxy.

(4) A₁ is —(C═O)— and B₁ is C₁-C₆alkyl, (phenyl)C₀-C₂alkyl, or (heterocycloalkyl)C₀-C₂alkyl, each of which is substituted with from 0 to 2 substituents independently chosen from halogen, hydroxyl, amino, cyano, C₁-C₄alkyl, C₁-C₄alkoxy, mono- and di-(C₁-C₄alkyl)amino, (C₃-C₇cycloalkyl)C₀-C₂alkyl, and (heterocycloalkyl)C₀-C₂alkyl.

Ester conjugate prodrugs of 2,6-HNK may be prepared as follows. The ester conjugate prodrugs shown in this table may be used in the methods of treatment disclosed herein.

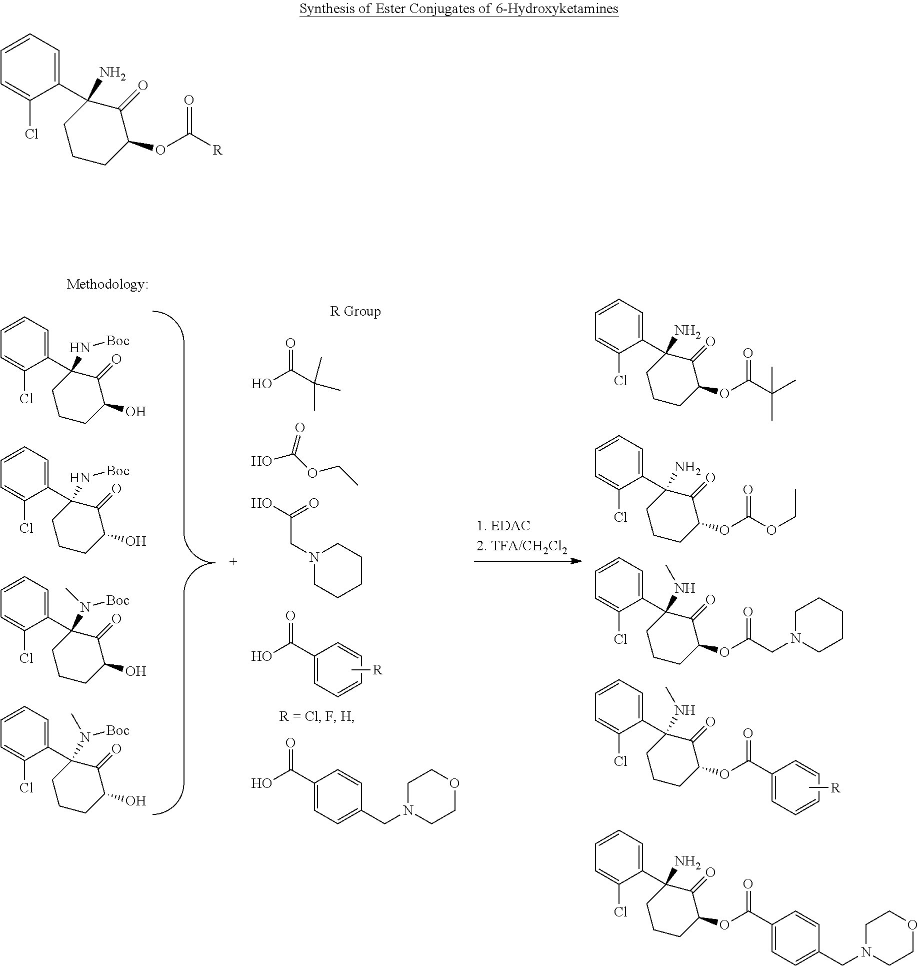

### Antidepressant and Anxiolytic Activity of (2S,6S)-HNK and (2R,6R)-HNK

This disclosure demonstrates the unique antidepressant effects of 2,6-HNK, particularly 2R,6R-HNK, and implicates a non-NMDAR inhibition-dependent mechanism. These findings reveal that 2,6-HNK, e.g., (2R,6R)-HNK, produces antidepressant-like behavioral effects, which require the activation of AMPA receptors. Considering the lack of side effects, and the favorable physiochemical properties of HNKs, these findings have establish the pharmacological effects of 2,6-HNK, e.g., 2R,6R-HNK. The disclosure also includes human and in vivo animal data showing 2,6-HNK, e.g., (2R,6R)-HNK, efficacy humans or in models of anxiety, anhedonia, suicidal ideation post-traumatic stress disorder, obsessive compulsive disorder, fatigue, and depression.

### Animal Methods

Male CD-1 mice (8-10 weeks old, Charles River Laboratories, Mass., USA) were housed in groups of four-five per cage with a constant 12-hour light/dark cycle (lights on/off at 07:00/19:00). Food and water were available ad libitum. Mice acclimatized to the new environment for seven days prior to the start of the experiments. For the whole-cell NMDA current electrophysiological recordings, male Sprague-Dawley rats (housed three per cage; Charles River, Wilmington, Mass.) were used. EPSC recording were done from rats at postnatal day 24-25. All experimental procedures were approved by the University of Maryland, Baltimore Animal Care and Use Committee and were conducted in full accordance with the National Institutes of Health Guide for the Care and Use of Laboratory Animals.

### Forced-Swim Test

Mice were tested in the FST 1 hour and/or 24 hours post-injection. During the FST, mice were subjected to a 6-min swim session in clear Plexiglass cylinders (30 cm height×20 cm diameter) filled with 15 cm of water (23±1° C.). The FST was performed in normal light conditions (800 Lux). Sessions were recorded using a digital video-camera. Immobility time, defined as passive floating with no additional activity other than that necessary to keep the animal's head above water, was scored for the last 4 min of the 6-min test by a trained observer blind to the treatment.

### Open Field Test

Mice were placed into individual open-field arenas (50 cm length×50 cm width×38 cm height; San Diego Instruments, San Diego, Calif., USA) for a 60-min habituation period. Mice were then injected with the respective drug and assessed for locomotor activity for another 60 min. Distance travelled was analyzed using TopScan v2.0 (CleverSys, Inc, Reston, Va., USA).

### Novelty-Suppressed Feeding

Mice were singly housed and food-deprived for twenty-four hours in freshly-made home-cages. Two normal chow diet pellets were placed on a square food platform (10×10 cm) in the center of an open-field arena (40×40 cm). Thirty or sixty min after drug administration, mice were introduced into a corner of the arena. The time needed for the mice to take a bite of food was recorded over a 10 min period by a trained observer blind to the treatment groups. After the test, the mice were returned to their home cage containing pre-weighed food pellets, and latency to bite the food as well as consumption was recorded for a period of 10 min.

### Learned Helplessness

The LH paradigm consisted of three different phases, i.e., inescapable shock training, LH screening, and the LH test. For the inescapable shock portion of the test (Day 1), the animals were placed in one side of two-chambered shuttle boxes (34 cm height×37 cm width×18 cm depth; Coulbourn Instruments, PA, USA), with the door between the chambers closed. Following a five-min adaptation period, 120 inescapable foot-shocks (0.45 mA, 15 sec duration, randomized average inter-shock interval of 45 sec) were delivered through the grid floor. During the screening session (Day 2), the mice were placed in one of the two chambers of the apparatus for 5 min. A shock (0.45 mA) was then delivered, and the door between the two chambers was raised simultaneously. Crossing over into the second chamber terminated the shock. If the animal did not cross over, the shock terminated after 3 sec. A total of 30 screening trials of escapable shocks were presented to each mouse with an average of 30 sec delay between each trial. Mice that developed helplessness behavior (>5 escape failures during the last 10 screening shocks) were administered with the respective drug 24 hours following screening (Day 3). During the LH test phase (Day 4), the animals were placed in the shuttle boxes and, after a 5-min adaptation period, a 0.45 mA shock was delivered concomitantly with door opening for the first five trials, followed by a 2-sec delay for the next 40 trials. Crossing over to the second chamber terminated the shock. If the animal did not cross over to the other chamber, the shock was terminated after 24 sec. A total of 45 trials of escapable shock were presented to each mouse with 30 sec inter-trial intervals. The number of escape failures was recorded for each mouse.

### Chronic Social Defeat Stress and Social Interaction

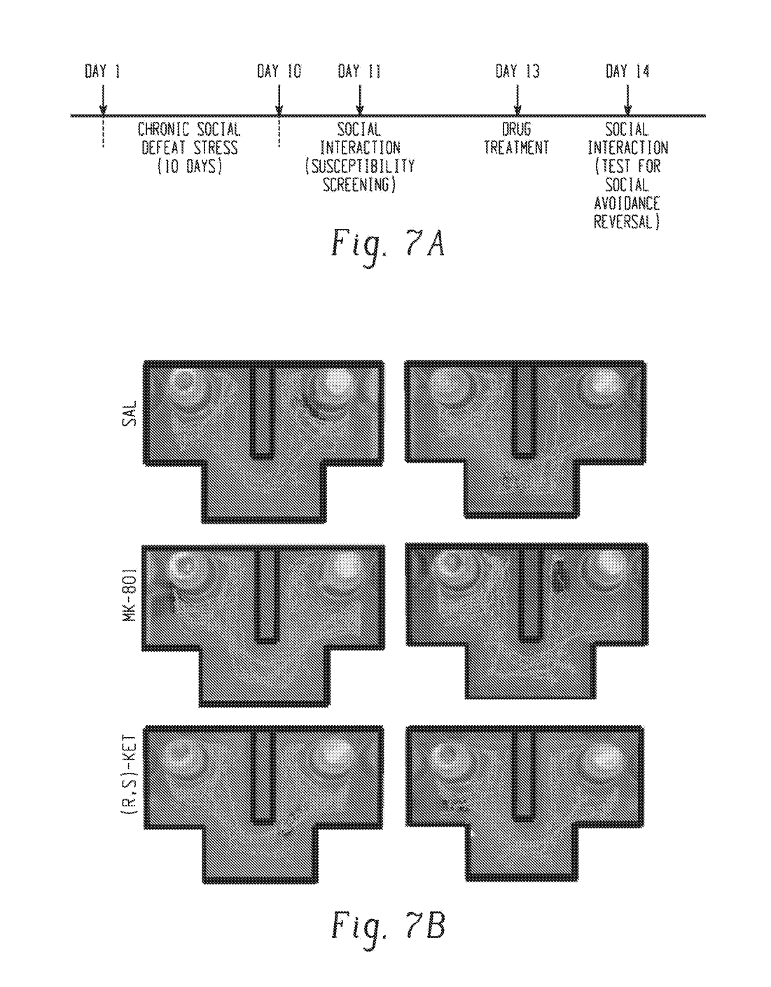

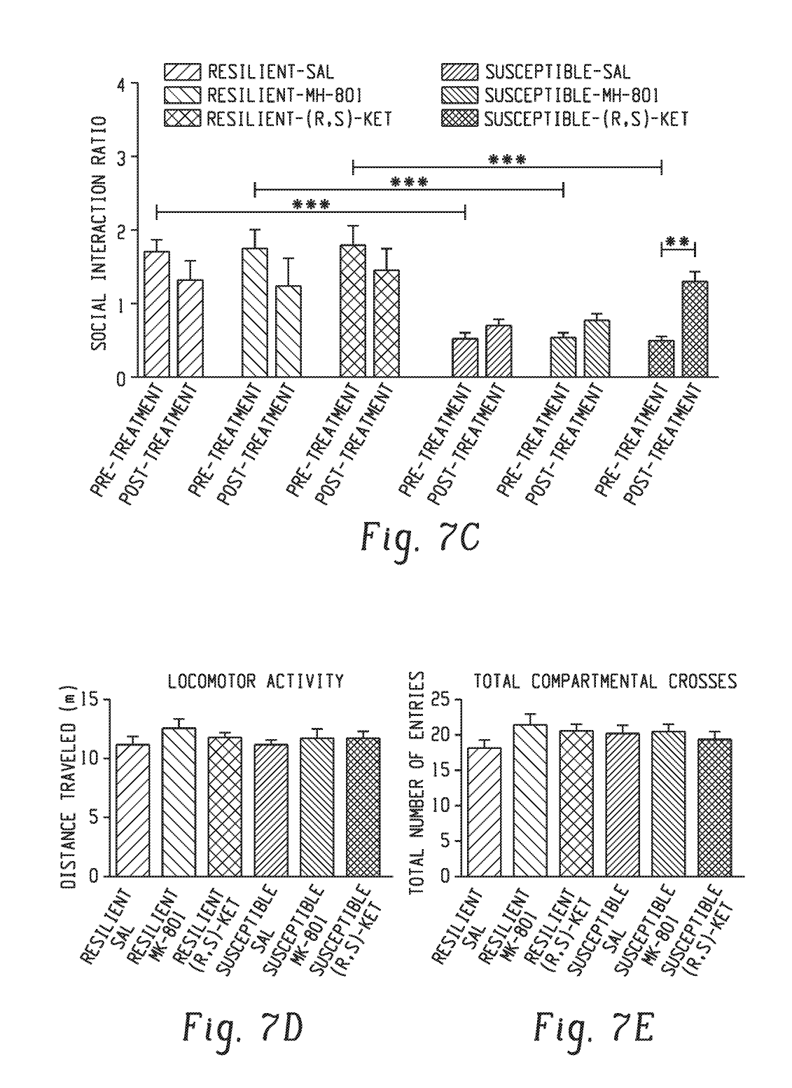

Male C57BL/6J mice underwent a 10-day chronic social defeat stress paradigm. Briefly, experimental mice were introduced to the home cage (43 cm length×11 cm width×20 cm height) of a resident aggressive retired CD-1 breeder, prescreened for aggressive behaviors, for 10 min. Following this physical attack phase, mice were transferred and housed in the opposite side of the resident's cage divided by a Plexiglas perforated divider, in order to maintain continuous sensory contact. This process was repeated for 10 days. Experimental mice were introduced to a novel aggressive CD-1 mouse each day. On day 11, test mice were screened for susceptibility in a social interaction/avoidance choice test. The social interaction apparatus consisted of a rectangular three-chambered box (mouse conditioned-place preference chamber; Stoelting Co., Wood Dale, Ill., USA), see FIG. 7b) comprised of two equal sized end-chambers and a smaller central chamber. The social interaction/avoidance choice test consisted of two 5-min phases. During the habituation phase, mice explored the empty apparatus. During the test phase, two small wire cages (Galaxy Cup, Spectrum Diversified Designs, Inc., Streetsboro, Ohio, USA), one containing a “stranger” CD-1 mouse and the other one empty, were placed in the far corners of each chamber. The time spent interacting (nose within close proximity of the cage) with the “stranger” mouse versus the empty cage was analysed using TopScan video tracking software (CleverSys, Reston, Va.). Locomotor activity (total distance moved over 5 min) and number of total crosses into and out of the central chamber were also measured. The social interaction ratio was calculated by dividing the time spent interacting with the “stranger” by the time spent with the empty cage. Mice having a social interaction ratio higher than 1.0 were considered resilient and mice with a social interaction ratio lower than 1.0 were considered susceptible. On day 13 resilient and susceptible mice received an i.p. injection of either saline, (R,S)-KET (20 mg/kg; chosen based upon dose previously effective in C57BL/6J mice³¹), MK-801 (0.1 mg/kg) or (2R,6R)-HNK (20 mg/kg). Mice were re-tested for social interaction/avoidance on day 15 (24 hours following treatment).

### Pre-Pulse Inhibition

Mice were individually tested in acoustic startle boxes (SR-LAB, San Diego Instruments). Following drug administration, mice were placed in the startle chamber for a 30-min habituation period. The experiment started with a further 5-min adaptation period during which the mice were exposed to a constant background noise (67 dB), followed by five initial startle stimuli (120 dB, 40 msec duration each). Subsequently, animals were exposed to five different trial types: pulse alone trials (120 dB, 40 msec duration), three pre-pulse trials of 76, 81 and 86 dB of white noise bursts (20 msec duration) preceding a 120 dB pulse by 100 msec, and background (67 dB) no-stimuli trials. Each of these trials was randomly presented five times. Ketamine's dose selection (30 mg/kg) was based on a dose-response study we performed in a previous study. The percentage pre-pulse inhibition (% PPI) was calculated using the following formula: [(magnitude on pulse alone trial—magnitude on pre-pulse+pulse trial)/magnitude on pulse alone trial]×100.

### Chronic Corticosterone-Induced Anhedonia Tests

### Sucrose Preference Test

For assessing the baseline sucrose preference, mice were singly housed for 24 hours and presented with two identical bottles containing either tap water or 1% sucrose solution. Following baseline sucrose measurement, mice were re-grouped housed (5 mice per cage) and treated for 4 weeks with corticosterone (25 μg/ml equivalent) given in water bottles. Prior to initiation of any behavioral measurements, animals were weaned off corticosterone treatment; 3 days corticosterone 12.5 μg/ml and 3 days corticosterone 6.25 μg/ml, followed by 1 week of complete withdrawal from the drug. Mice were subsequently singly-housed in freshly-made home cages and provided with two bottles containing either tap water or 1% sucrose solution. Twenty-four hours later, mice that developed anhedonia phenotype (<55% sucrose preference) were treated with saline or (2R,6R)-HNK (10 mg/kg) and sucrose preference measured after an additional 24 hours.

### Female Urine Sniffing Test

A separate cohort of mice were treated with the same chronic corticosterone administration paradigm as described above, and 24 hours later assessed for female urine sniffing preference as a measure of hedonic behavior. Mice were singly-housed in freshly-made home cages for a habituation period of 10 min. Subsequently, one plain cotton tip was secured on the center of the cage wall and mice were allowed to sniff and habituate to the tip for a period of 30 min. Then, the plain cotton tip was removed and replaced by two cotton tip applicators one infused with fresh female mouse estrus urine and the other with fresh male mouse urine. These applicators were presented at the same time and secured at the two corners of the cage wall. Sniffing time for both the female and male urine was scored by a trained observer for a period of three minutes. Twenty-four hours later, mice that developed anhedonia phenotype (<55% female urine preference; susceptible phenotype), as well as mice that did not develop anhedonia phenotype (>65% female urine preference; resilient phenotype) were treated with either saline or (2R,6R)-HNK (10 mg/kg) and re-tested for female urine preference 24 hours later.

### Rotarod

The rotarod test was conducted to compare the effects of ketamine, (2S,6S)-HNK and (2R,6R)-HNK on motor coordination. The experiment consisted of two phases: training phase (4 days) and a test phase (1 day). On each of the training days five trials (trial time: 3 min) were conducted with an inter-trial interval of two min Mice were individually placed on the rotarod apparatus (HTC Life Science; Woodland Hills, Calif., USA) and the rotor (3.75 inch diameter) accelerated from 5-20 RPM over a period of three minutes. Latency to fall was recorded for each trial. Animals with an average of <100 sec of latency to fall during the last training day were excluded from the experiment. On the test day (day 5), mice received (i.p.) injections of saline, (R,S)-KET (10 mg/kg), (2S,6S)-HNK (25 or 125 mg/kg) or (2R,6R)-HNK (2.5 or 125 mg/kg) and were tested in the rotating rod 5-. 10-, 15-, 20-, 30- and 60-min post-injection using the same procedure described for the training days.

### Drug Discrimination

Mice were food restricted until they reached 85% of their initial body weight and were maintained at 85% throughout the duration of the experiment Animals were trained to lever press for food (20 mg sucrose pellets; TestDiet, St. Luis, Mo., USA) in standard two lever-operant conditioning chambers (Coulbourn Instruments, Whitehall, Pa., USA), under a fixed-ratio 5 of reinforcement (FR5) in daily 30-min sessions. When stable responding was succeeded over 3 consecutive sessions (average of 40 training sessions), mice were trained to discriminate ketamine (10 mg/kg) from saline (7.5 ml/kg) under a double alternation schedule (e.g., ketamine, ketamine, saline, saline). The subjects received either ketamine (10 mg/kg; i.p.) or saline (7.5 ml/kg) 15 minutes prior to the start of the 30-minute session. Responding to the correct lever resulted in the delivery of a reward, while incorrect responding reset the FR for correct lever-responding. Drug discrimination test sessions were conducted when mice reached the following criteria: (1) first FR5 completed on the correct lever, and (2)≥85% correct lever responding over the entire session. During the test sessions mice were administered with saline (7.5 ml/kg), ketamine (10 mg/kg), phencyclidine (PCP; 3 mg/kg) or (2R,6R)-HNK (10 and 50 mg/kg). At this stage completion of a FR5 on either lever resulted in the delivery of food reward. Recording of responses and pellet delivery were controlled and calculated by an automated computer system (Graphic State v3.1; Coulbourn Instruments, Whitehall, Pa., USA).

### Electroencephalogram (EEG) Experiments

### Surgery

EEG experiments were performed according to Raver et al., (Neuropsychopharmacology, 38, 2338-2347 (2013)) with minor modifications. Mice were anesthetized with isoflurane and kept under anesthesia throughout the surgery. An F20-EET radio-telemetric transmitter (Data Sciences International, Minneapolis, Minn.) was implanted subcutaneously and its leads implanted over the dura above the frontal cortex (1.7 mm anterior to bregma) and the cerebellum (6.4 mm posterior to bregma). Animals recovered from surgery for 7 days before recordings.

### EEG Recordings

Mice were singly housed and acclimated to the behavioral room for 24 hours prior to EEG recordings. EEGs were recorded using the Dataquest A.R.T. acquisition system (Data Sciences International) with frontal EEG recordings referenced to the cerebellum. Baseline EEG (10 min) recordings were followed by an i.p. injection of saline, ketamine (10 mg/kg) or (2R,6R)-HNK (10 mg/kg) and 40 min of post-injection recordings.

### In Vivo Data Analysis

ECoGs were analyzed using custom-written MATLAB scripts (Version 2012a, Mathworks, Mass.) and the mtspecgramc routine in the Chronux Toolbox (http://chronux.org; Mitra and Bokil, 2008). Oscillation power in each bandwidth (δ=1-3 Hz; θ=4-7 Hz; α=8-12 Hz; β=13-29 Hz; γ=30-80 Hz) was computed in 10 min bins from spectrograms for each animal.

### Tissue Distribution and Clearance Measurements of Ketamine and Metabolites

Mice were euthanized by a 30-sec exposure to 3% isoflurane and decapitated at 10, 30, 60, 240 or 480 minutes following drug administration. Trunk blood was collected in EDTA-containing tubes and centrifuged at 8000 rpm for 6 min (4° C.). Plasma was collected and stored at −80° C. until analysis. Whole brains were simultaneously collected, rinsed with phosphate-buffered saline, immediately frozen in dry ice and stored at −80° C. until analysis.

The concentrations of ketamine and its metabolites in plasma and brain tissue were determined by achiral liquid chromatography-tandem mass spectrometry. For plasma samples, the calibration standards for (R,S)-ketamine, (R,S)-norketamine, (2R,6R;2S,6S)-HNK and (R,S)-DHNK ranged from 10,000 ng/ml to 19 ng/ml. The quantification of (R,S)-ketamine, (R,S)-norketamine, (R,S)-DHNK, and the HNK stereoisomers was accomplished by calculating area ratios using D₄-ketamine (10 μl of 10 μg/ml solution) as the internal standard. Whole brains were suspended in 990 μl of water:methanol (3:2, v/v), D₄-ketamine (10 μl of 10 μg/ml) added and the resulting mixture homogenized on ice with a polytron homogenizer and centrifuged at 21,000×g for 30 min. The supernatant was collected and processed using 1 ml Oasis HLB solid phase extraction cartridges (Waters Corp., Waltham, Mass.). The cartridges were preconditioned with 1 ml of methanol, followed by 1 ml of water and then 1 ml ammonium acetate [10 mM, pH 9.5]. The supernatants were added to the cartridges, followed by 1 ml of water and the compounds were eluted with 1 ml of methanol. The eluent was transferred to an autosampler vial for analysis. QC standards for the analysis of (R,S)-ketamine, (R,S)-norketamine, (R,S)-DHNK and (2R,6R;2S,6S)-HNK ranged from 10,000 ng/ml to 19 ng/ml, and quantification was accomplished using D4-(R,S)-ketamine as the internal standard. QC standards were prepared daily by adding 10 μl of the appropriate standard solution and 10 μl of internal standard solution (100 ng/ml) to methanol.

### Chemical Description

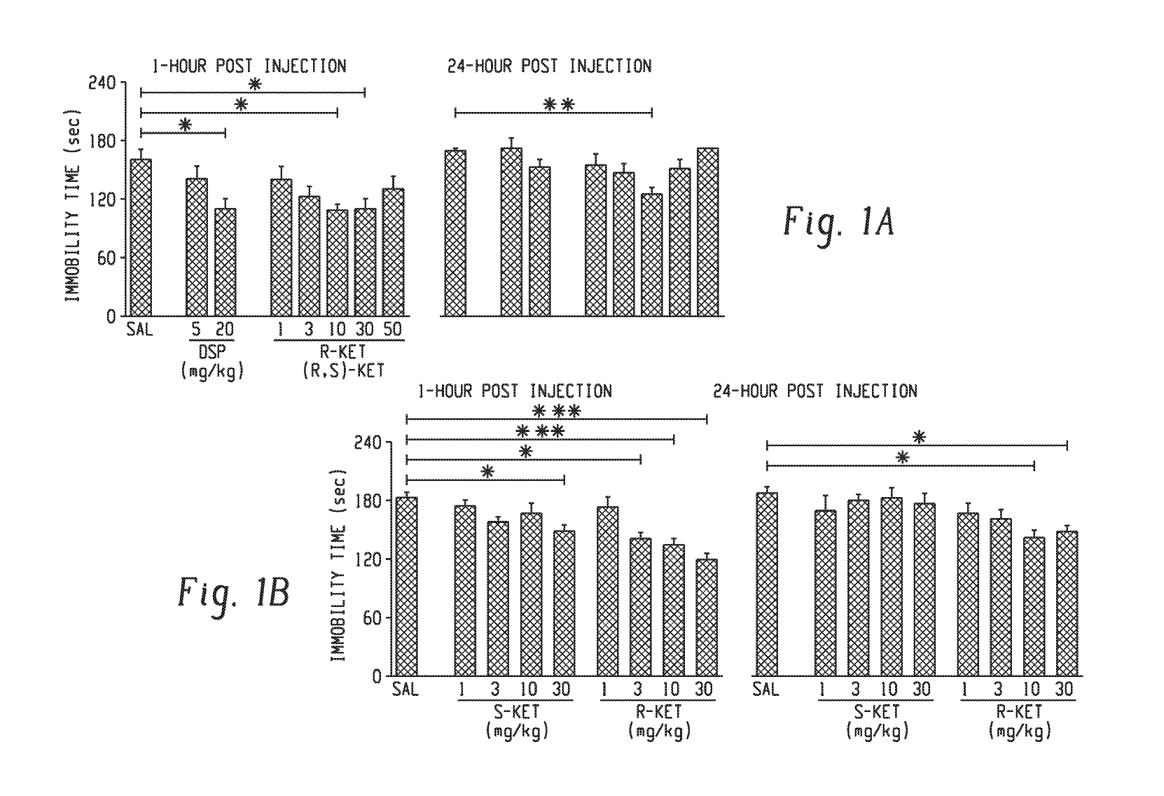

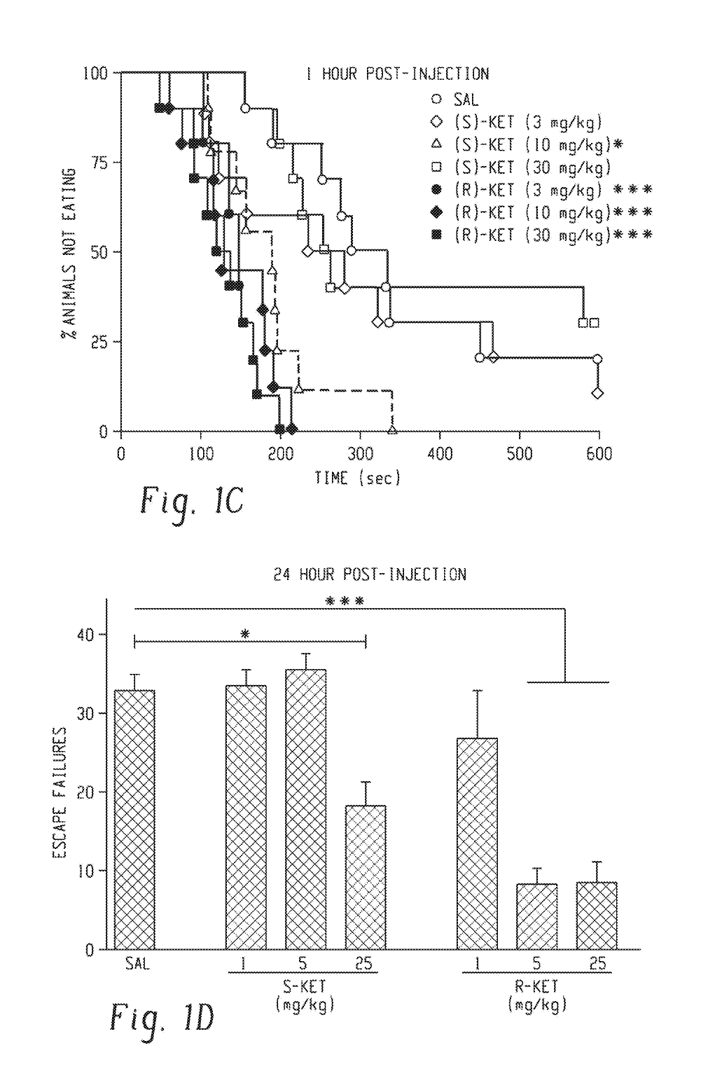

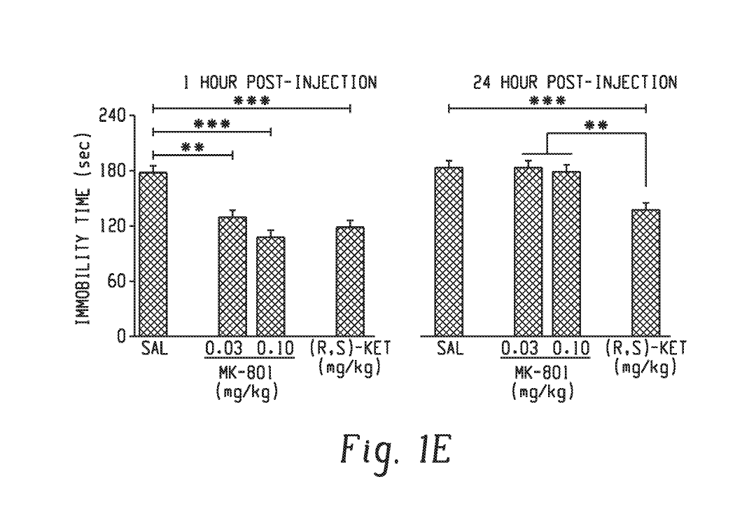

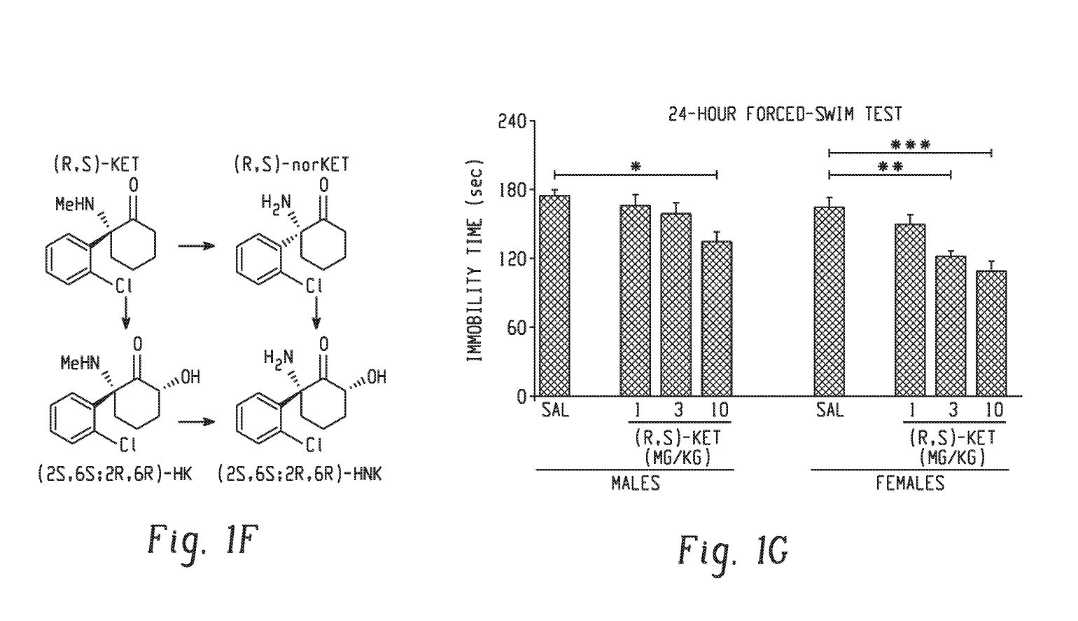

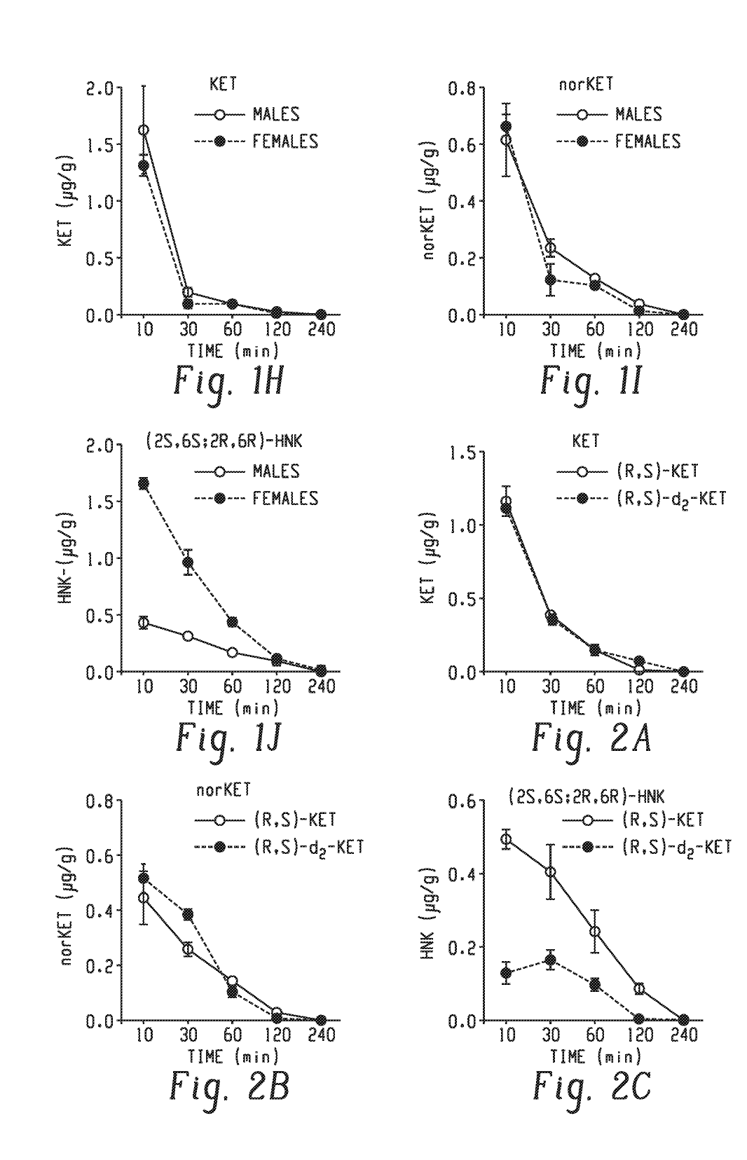

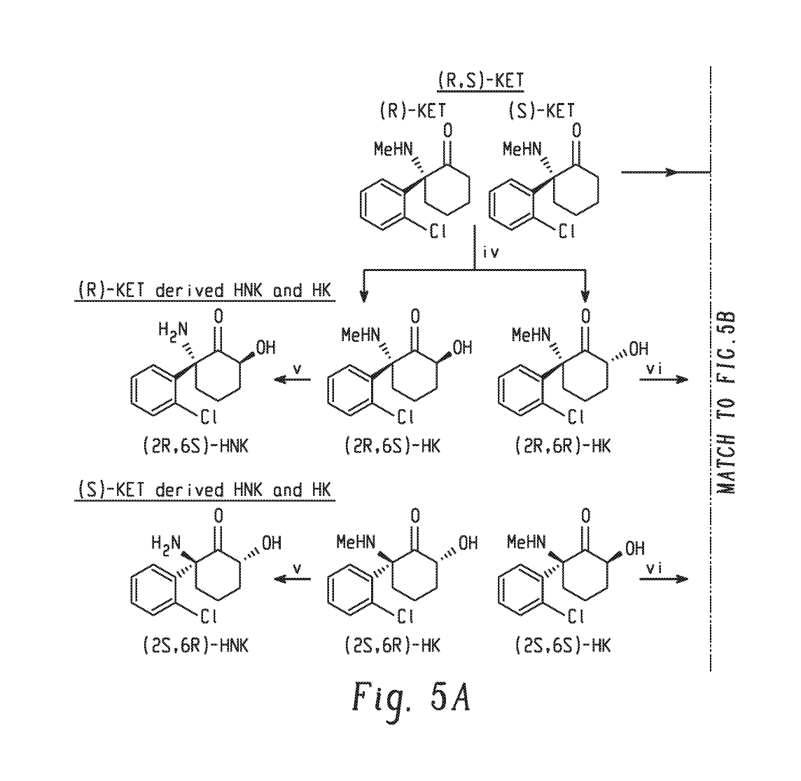

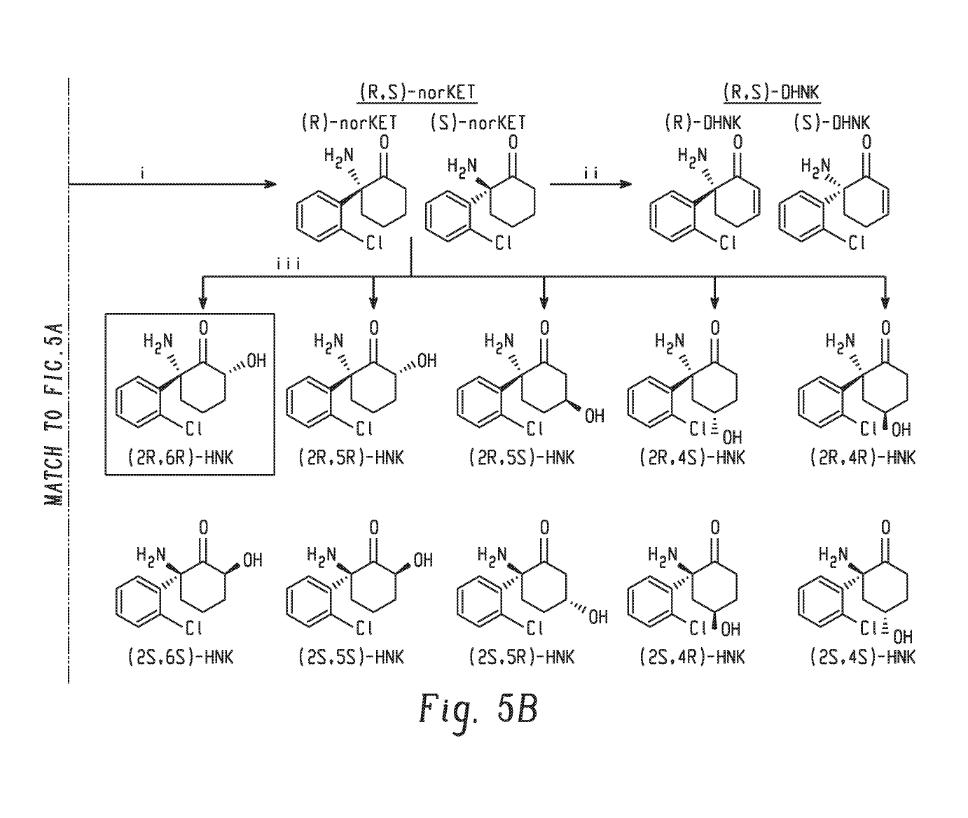

As shown in FIG. 1f and 5 ketamine is metabolized in vivo via P450 enzymatic transformations. (i) (R,S)-Ketamine (KET) is selectively demethylated to give (R,S)-norketamine (norKET). (ii) NorKET can be then dehydrogenated to give (R,S)-dehydroxynorketamine (DHNK). (iii) Alternatively, norKET can be hydroxylated to give the hydroxynorketamines (HNK). (iv) (R,S)-KET can also be hydroxylated at the 6-position to give either the E-6-hydroxyketamine ((2S,6R;2R,6S)-HK)) or Z-6-hydroxyketamine ((2S,6S;2R,6R)-HK)). (v) Demethylation of (2S,6S;2R,6R)-HK yields the production of (2S,6S;2R,6S)-hydroxynorketamine (HNK). (vi) Demethylation of (2S,6S;2R,6R)-HK further gives (2S,6S;2R,6R)-hydroxynorketamine (HNK). Abbreviations: DHNK, dehydroxynorketamine; HK, hydroxyketamine; HNK, hydroxynorketamine; KET, ketamine.

The structure of racemic (2,6)-hydroxynorketamine was reported by Leung and Baillie (J. Med. Chem., (1986) 29: 2396-2399). This compound is also known as (Z)-6-hydroxynorketamine.

The structure of (2R,6R)-hydroxynorketamine, also known by its IUPAC name, (2R,6R)-2-amino-2-(2-chlorophenyl)-6-hydroxycyclohexanone, is

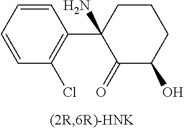 [SMILES: N[C@@]1(c2ccccc2Cl)CCC[C@@H](O)C1=O]

The structure of (2S,6S)-hydroxynorketamine, also known by its IUPAC name (2S,6S)-2-amino-2-(2-chlorophenyl)-6-hydroxycyclohexanone, is

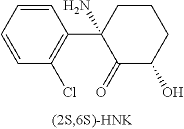 [SMILES: N[C@]1(c2ccccc2Cl)CCC[C@H](O)C1=O]

The disclosure includes all stereoisomers of hydroxynorketamine and dihydronorketamine

(2S,6S)-hydroxynorketamine and (2R,6R)-hydroxynorketamine are prepared according to the following synthetic schemes. In the discussion below the intermediates leading to (2R,6R-HNK) are given the numbers 2A, 3A, 4A, 5A, and 6A.

### Synthetic Route for (2S,6S)-HNK

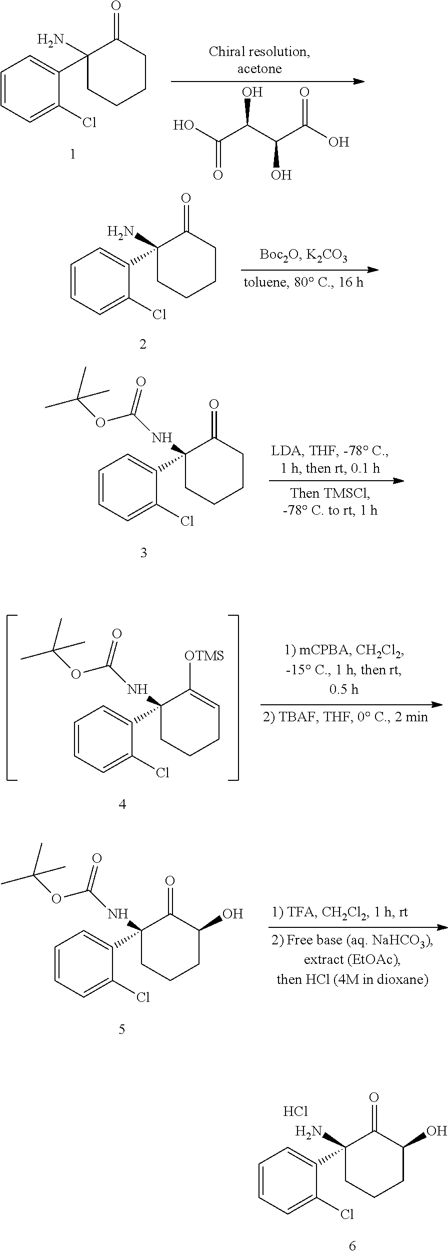

### Synthetic Route for 6,6-dideuteroketamine Hydrochloride

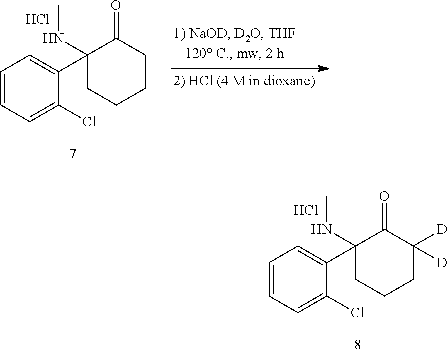 [SMILES: CNC1(c2ccccc2Cl)CCCCC1=O ; Cl ; [2H]C1([2H])CCCC(NC)(c2ccccc2Cl)C1=O ; Cl]

### Synthesis of 2R,6R-HNK and 2S,6S-HNK

### Chiral Resolution of (S)−)-Norketamine (2)

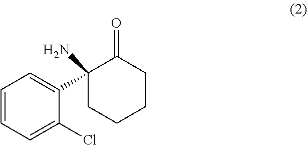 [SMILES: N[C@]1(c2ccccc2Cl)CCCCC1=O]

Racemic norketamine (22.7 grams, 101 mmol) (Cayman Chemicals, Ann Arbor, Mich., USA, prepared as described in Hong, S. C.& Davisson, J. N., J. Pharm. Sci. (1982) 71: 912-914) was dissolved in methanol (58 mL) and (2S,3S)-(D)-(−)-tartaric acid (17.1 grams) in methanol (227 mL) was added. The reaction was stirred at room temperature for 16 hours. The solvent was partially removed by rotary evaporation. 2-Butanone was added (100 mL) and the solvent was further removed by rotary evaporation to give the solid norketamine D-tartrate. The solid material was dissolved in 6.0 L of refluxing acetone. The reaction mixture was filtered, and allowed to cool to room temperature without stirring for two days. Fine needle-like low density crystals were collected to give 6.0 grams of S-norketamine D-tartrate. The filtrate was saved for later isolation of the other enantiomer. The (S)-norketamine D-tartrate was recrystallized from hot acetone a further three times to improve the enantiopurity, resulting in 3.2 grams of the (S)-norketamine D-tartrate. The optical rotation was measured and compared to literature values to confirm the absolute stereochemistry, while enantiomeric excess was determined to be >97% by chiral HPLC. S-Norketamine D-tartrate was then converted into the free base by treatment with aqueous sodium hydroxide and extraction with ethyl acetate. The organic phase was taken and the solvent removed by rotary evaporation to give (S)-norketamine (2) as a white crystalline solid. ¹H NMR spectra matched reported spectra. The free base was formed by treatment of the tartrate salt with 1N aqueous sodium hydroxide, extraction with ethyl acetate, and removal of the organic solvent by rotary evaporation.

Chiral HPLC: 97% ee. (Chiralpak AD, 60% ethanol in hexanes, 1 mL/min, rt: 5.01 min.)

[α]D²⁰: (+)-55° (c1.0, H₂O, D-tartrate salt) compared to (+)-57 degrees (c 2.0, H₂O, D-tartrate salt).

### Chiral Resolution of (R)-Norketamine (2A)

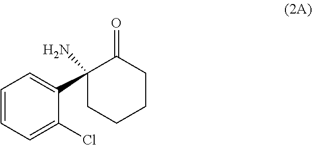 [SMILES: N[C@@]1(c2ccccc2Cl)CCCCC1=O]

(R)-Norketamine (2A) was produced in an analogous fashion to that of (S)-norketamine, except that (2R,3R)-(L)-(+)-tartaric acid was used as a chiral resolution agent instead of (2S,3S)-(D)-(−)-tartaric acid. Chiral HPLC: 98% ee. (Chiralpak AD, 60% ethanol in hexanes, 1 mL/min, rt: 6.83 min.) [a[_{D}²⁰: (−)-53° (c 1.0, H₂O, L-tartrate salt)

### Synthesis of (S)-tert-Butyl (1-(2-chlorophenyl)-2-oxocyclohexyl)carbamate (3)

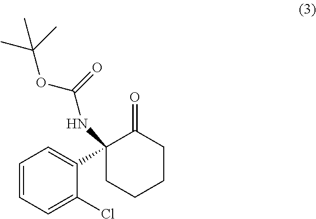 [SMILES: CC(C)(C)OC(=O)N[C@]1(c2ccccc2Cl)CCCCC1=O]

To a solution of (S)-norketamine (2) (1.85 g, 8.27 mmol) in toluene (100 mL) was added potassium carbonate (3.43 g, 24.8 mmol) and BOC-anhydride (2.71 g, 12.4 mmol). The reaction was heated to 80° C. and stirred for 16 hours. The reaction was then cooled, extracted with ethyl acetate and washed with water. The organic layer was taken and the solvent removed in vacuo to give the crude product. Purification by silica gel chromatography (0% to 60% ethyl acetate in hexanes) gave the final product (3) as a white solid.

¹H NMR (400 MHz, CDCl₃) δ 7.83 (d, J=8.0 Hz, 1H), 7.42-7.28 (m, 2H), 7.28-7.13 (m, 1H), 6.59 (s, 1H), 3.83 (d, J=14.3 Hz, 1H), 2.45-2.36 (m, 1H), 2.36-2.25 (m, 1H), 2.04 (ddq, J=11.5, 5.5, 3.0 Hz, 1H), 1.89-1.56 (m, 4H), 1.29 (s, 9H).

¹³C NMR (101 MHz, CDCl₃) δ 209.0, 153.4, 135.1, 133.7, 131.5, 130.9, 129.2, 126.2, 79.0, 67.1, 39.4, 38.4, 30.8, 28.2, 22.3.

HRMS (ESI+): Expected 346.1186 [M+Na]⁺ (C₁₇H₂₂ClNO₃Na). Observed 346.1180. [α]D²⁰: (+)-39.5° (C═1.0, CH₂Cl₂).

### Synthesis of (R)-tert-Butyl (1-(2-chlorophenyl)-2-oxocyclohexyl)carbamate (3A)

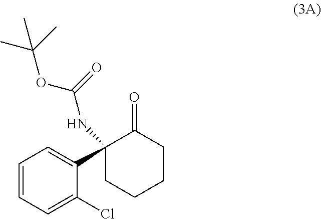 [SMILES: CC(C)(C)OC(=O)N[C@@]1(c2ccccc2Cl)CCCCC1=O]

The title compound was prepared in an analogous fashion to (S)-tert-butyl (1-(2-chlorophenyl)-2-oxocyclohexyl)carbamate (3), utilizing (R)-norketamine instead of (S)-norketamine.

¹H NMR (400 MHz, CDCl₃) δ 7.85 (d, J=8.0 Hz, 1H), 7.34 (dd, J=8.0, 1.4 Hz, 2H), 7.30-7.21 (m, 1H), 6.61 (s, 1H), 3.84 (d, J=14.4 Hz, 1H), 2.47-2.37 (m, 1H), 2.38-2.29 (m, 1H), 2.09-2.02 (m, 1H), 1.86-1.62 (m, 4H), 1.31 (s, 9H).

¹³C NMR (101 MHz, CDCl₃) δ 209.0, 153.4, 135.0, 133.7, 131.5, 130.8, 129.2, 126.2, 79.0, 67.1, 39.4, 38.4, 30.8, 28.2, 22.3.

HRMS (ESI+): Expected 346.1186 [M+Na]⁺ (C₁₇H₂₂ClNO₃Na). Observed 346.1188. [α]D²⁰: (−)-60.7° (c1.0, CH₂Cl₂).

### Synthesis of tert-Butyl ((1S,3S)-1-(2-chlorophenyl)-3-hydroxy-2-oxocyclohexyl)carbamate

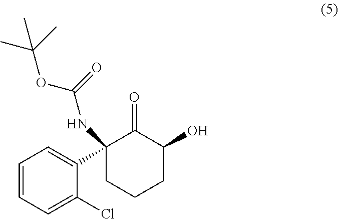 [SMILES: CC(C)(C)OC(=O)N[C@]1(c2ccccc2Cl)CCC[C@H](O)C1=O]

A solution of (S)-tert-butyl (1-(2-chlorophenyl)-2-oxocyclohexyl)carbamate 3 (6.5 grams, 20.1 mmol) in THF (100 mL), was cooled to −78° C. under a nitrogen atmosphere. Lithium diisopropylamide (2.0 M in THF/heptane/ethylbenzene, 26 mL, 2.6 eq. 52.2 mmol) was added by syringe. The reaction was stirred 1 hour at −78° C., then allowed to warm to room temperature for 5 minutes. The reaction was cooled to −78° C., and chlorotrimethylsilane (5.7 grams, 2.6 eq., 52.2 mmol) was added as a neat liquid by syringe. The reaction was stirred for 30 minutes at −78° C., and then allowed to warm to room temperature over 30 minutes. The reaction was then quenched by being poured into aqueous saturated ammonium chloride. Ethyl acetate was added to the resulting mixture, the organic phase was separated and the solvent was removed by rotary evaporation to give the crude enol ether 4 as a solid which was immediately used without further purification. The enol ether 4 (7.8 grams) was dissolved in dichloromethane (100 mL) and cooled to −15° C. (ice-lithium chloride), under a nitrogen atmosphere. 3-Chloroperbenzoic acid (5.0 grams, 1.1 eq.) was then added as a solid. The reaction was stirred for 1 hour at −15° C., then the temperature was raised to room temperature and an additional 100 mL of dichloromethane was added. The reaction was stirred a further 0.5 hours. The reaction was then quenched by being poured into a 50/50 mixture of saturated aqueous sodium thiosulfate and saturated aqueous sodium bicarbonate. The reaction was extracted into dichloromethane and the solvent removed by rotary evaporation. Then tetrahydrofuran (100 mL) was added to the crude material. The reaction was cooled to −5° C., and tetrabutylbutyl ammonium fluoride (1.0 M in THF, 25 mL, 1.2 eq. was added). The reaction was stirred for 2 minutes, before being quenched by addition to saturated aqueous sodium bicarbonate. Extraction into ethyl acetate, followed by removal of the solvent by rotary evaporation gave the crude final product 5. Purification by silica gel chromatography (0% to 70% ethyl acetate in hexanes), gave the purified final product as a solid.

¹H NMR (400 MHz, CDCl₃) δ 7.80 (d, J=7.9 Hz, 1H), 7.34 (ddd, J=8.8, 7.1, 1.4 Hz, 2H), 7.29-7.18 (m, 1H), 6.60 (s, 1H), 4.12 (dd, J=11.8, 6.7 Hz, 1H), 3.87 (d, J=14.3 Hz, 1H), 3.38 (s, 1H), 2.36 (ddq, J=13.1, 6.5, 3.2 Hz, 1H), 1.74 (ddt, J=7.8, 5.7, 2.8 Hz, 2H), 1.69-1.59 (m, 1H), 1.59-1.40 (m, 1H), 1.30 (s, 9H).

¹³C NMR (100 MHz, CDCl₃) δ 209.9, 153.3, 134.1, 133.8, 131.4, 131.0, 129.7, 126.3, 79.4, 72.4, 66.7, 40.4, 38.8, 28.2, 19.6.

HRMS (ESI+): Expected 362.1135 [M+Na]⁺ (C₁₇H₂₂ClNO₄Na). Observed 362.1134. [α]_{D}²⁰: (+)-60.7° (c 1.0, CHCl₃).

### Synthesis of tert-Butyl ((1R,3R)-1-(2-chlorophenyl)-3-hydroxy-2-oxocyclohexyl)carbamate (5A)

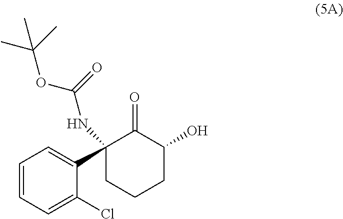 [SMILES: CC(C)(C)OC(=O)N[C@@]1(c2ccccc2Cl)CCC[C@@H](O)C1=O]

The title compound was prepared in an analogous fashion to (tert-butyl ((1S,3S)-1-(2-chlorophenyl)-3-hydroxy-2-oxocyclohexyl)carbamate 5 by utilizing (R)-tert-butyl (1-(2-chlorophenyl)-2-oxocyclohexyl)carbamate instead of the S-enantiomer.

¹H NMR (400 MHz, CDCl₃) δ 7.80 (d, J=7.9 Hz, 1H), 7.34 (dd, J=8.5, 6.9 Hz, 2H), 7.32-7.21 (m, 1H), 6.60 (s, 1H), 4.12 (ddd, J=11.5, 8.9, 6.3 Hz, 1H), 3.92-3.83 (m, 1H), 3.37 (d, J=6.5 Hz, 1H), 2.36 (ddq, J=13.0, 6.5, 3.2 Hz, 1H), 1.74 (dq, J=6.4, 3.2, 2.5 Hz, 2H), 1.63 (dq, J=16.8, 9.2, 8.2 Hz, 1H), 1.59-1.40 (m, 1H), 1.30 (s, 9H).

¹³C NMR (100 MHz, CDCl₃) δ 209.9, 153.3, 134.1, 133.8, 131.4, 131.0, 129.7, 126.3, 79.4, 72.4, 66.7, 40.4, 38.8, 28.2, 19.5.

HRMS (ESI+): Expected 362.1135 [M+Na]⁺ (C₁₇H₂₂ClNO₄Na). Observed 362.1134. [α]_{D}²⁰: (−)-63.7° (c1.0, CHCl₃).

### Synthesis of (2S,6S)-2-amino-2-(2-chlorophenyl)-6-hydroxycyclohexanone hydrochloride ((2S,6S)-(+)-hydroxynorketamine hydrochloride) (6)

### HCl

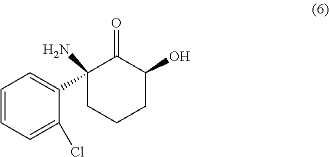 [SMILES: N[C@]1(c2ccccc2Cl)CCC[C@H](O)C1=O]

To a solution of tert-butyl ((1S,3S)-1-(2-chlorophenyl)-3-hydroxy-2-oxocyclohexyl)carbamate 5 (4.85 grams) in dichloromethane (10 mL) was added trifluoroacetic acid (11.0 mL, 10 eq.). The reaction was stirred at room temperature for 1 hour. The solvent and trifluoroacetic acid (TFA) were then removed by rotary evaporation. The resulting TFA salt was dissolved in water, washed with a 50/50 mixture of saturated aqueous sodium bicarbonate and saturated aqueous potassium carbonate solution, and extracted with ethyl acetate (2×) to give the free base. The ethyl acetate was removed by rotary evaporation. Ethyl acetate (4 mL) was added and HCl in dioxane (4.0 M, 6.0 mL) was added. A white solid immediately precipitated. The suspension was agitated for 30 seconds and then the solid was filtered off and dried under vacuum to give the desired final product.

¹H NMR (400 MHz, MeOD) δ 7.92-7.81 (m, 1H), 7.66-7.50 (m, 3H), 4.28 (dd, J=11.7, 6.6 Hz, 1H), 3.19 (dd, J=14.0, 3.0 Hz, 1H), 2.30 (dddd, J=12.2, 6.6, 4.1, 2.3 Hz, 1H), 1.80-1.70 (m, 2H), 1.68-1.52 (m, 2H).

¹³C NMR (100 MHz, MeOD): δ 206.8, 134.0, 132.1, 131.6, 130.5, 130.0, 128.3, 73.0, 67.0, 38.4, 37.1, 18.7.

Chiral HPLC: 98.3% ee (Chiralpak AD column, 60% ethanol in hexanes, 1.0 mL/min, rt=6.0 min.)

HRMS (ESI+): Expected 240.0786 [M+H]⁺ (C₁₂H₁₅ClNO₂). Observed 240.0782. [α]_{D}²⁰: (+)-95° (c 1.0, H₂O).

### Synthesis of (2R,6R)-2-amino-2-(2-chlorophenyl)-6-hydroxycyclohexanone hydrochloride ((2R,6R)-(−)-hydroxynorketamine hydrochloride) (6A)

### HCl

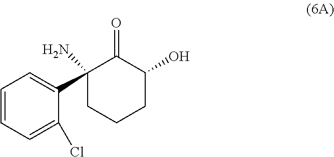 [SMILES: N[C@@]1(c2ccccc2Cl)CCC[C@@H](O)C1=O]

The title compound was prepared in an analogous fashion to that of (2S,6S)-(+)-hydroxynorketamine hydrochloride (6) by utilizing tert-butyl ((1R,3R)-1-(2-chlorophenyl)-3-hydroxy-2-oxocyclohexyl)carbamate instead of the S,S-enantiomer.

¹H NMR (400 MHz, MeOD): δ 7.94-7.83 (m, 1H), 7.62-7.53 (m, 3H), 4.29 (dd, J=11.6, 6.7 Hz, 1H), 3.19 (dd, J=14.0, 3.0 Hz, 1H), 2.30 (dddd, J=12.2, 6.6, 4.1, 2.3 Hz, 1H), 1.99-1.82 (m, 2H), 1.82-1.56 (m, 2H) ppm.

¹³C NMR (100 MHz, MeOD): δ 206.8, 134.0, 132.1, 131.6, 130.5, 130.1, 128.3, 73.3, 67.0, 38.4, 37.2, 18.7 ppm.

Chiral HPLC: 98.3% ee (Chiralpak AD column, 60% ethanol in hexanes, 1.0 mL/min, rt=7.9 min)

HRMS (ESI+): Expected 262.0605 [M+Na]⁺ (C₁₂H₁₄ClNO₂Na). Observed 262.0605 [α]D²⁰: (−)-92° (C═1.0, H₂O).

### Synthesis of 2-(2-Chlorophenyl)-6,6-Dideutero-2-(Methylamino)Cyclohexanone Hydrochloride (6,6-Dideuteroketamine Hydrochloride) (8)

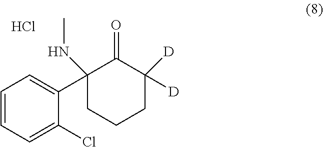 [SMILES: [2H]C1([2H])CCCC(NC)(c2ccccc2Cl)C1=O ; Cl]

Sodium deuteroxide (30% in deuterium oxide, 3.0 mL) was added to a solution of racemic ketamine hydrochloride (0.80 grams, 2.9 mmol) in a mixture of tetrahydrofuran (8.0 mL) and deuterium oxide (3.0 mL). The reaction was heated by microwave irradiation in a sealed vial to 120° C. for 2 hours. The reaction was cooled, extracted with ethyl acetate and washed with saturated aqueous sodium bicarbonate. The organic phase was taken and the solvent removed by rotary evaporation to give the crude product. Purification by reverse phase liquid chromatography (5% to 95% acetonitrile in water with 0.1% trifluoroacetic acid) gave the purified TFA salt. The free base was formed and isolated by washing the TFA salt with saturated aqueous sodium bicarbonate and extraction with ethyl acetate. The HCl salt was formed by the addition of HCl (4.0 M in dioxane), and filtration of the resulting white solid, to provide the title compound as a white solid.

¹H NMR (400 MHz, MeOD): δ 7.94-7.88 (m, 1H), 7.66-7.57 (m, 3H), 3.41-3.34 (m, 1H), 2.38 (s, 3H), 2.27-2.20 (m, 1H), 1.93-1.83 (m, 2H), 1.83-1.69 (m, 2H).

¹³C NMR (100 MHz, MeOD): δ 208.6, 136.1, 134.1, 133.6, 133.5, 129.9, 129.4, 73.8, 40.3 (septet, J_{C-D}=21 Hz, 1C), 37.6, 31.2, 28.1, 23.0.

HRMS (ESI+): Expected 240.1119 [M+H]+, (C₁₃H₁₅D₂ClNO). Observed 240.1120

### X-Ray Crystallography of (2S,6S)-Hydroxynorketamine Hydrochloride

The single crystal X-ray diffraction studies were carried out on a Bruker Kappa APEX-II CCD diffractometer equipped with Mo K_{α} radiation (λ=0.71073 Å). Crystals of the subject compound were grown by slow evaporation of a 50/50 Dichloroethane/Methanol solution. A 0.227×0.215×0.106 mm piece of a colorless block was mounted on a Cryoloop with Paratone oil. Data were collected in a nitrogen gas stream at 100(2) K using ϕ and ω̅ scans. Crystal-to-detector distance was 40 mm and exposure time was 5 seconds per frame using a scan width of 2.0°. Data collection was 100% complete to 25.00° in θ. A total of 9466 reflections were collected covering the indices, -9<=h<=9, -9<=k<=9, -14<=1<=14. 2949 reflections were found to be symmetry independent, with a Rᵢₙₜ of 0.0376. Indexing and unit cell refinement indicated a primitive, monoclinic lattice. The space group was found to be P2₁. The data were integrated using the Bruker SAINT software program and scaled using the SADABS software program. Solution by direct methods (SHELXT) produced a complete phasing model consistent with the proposed structure.

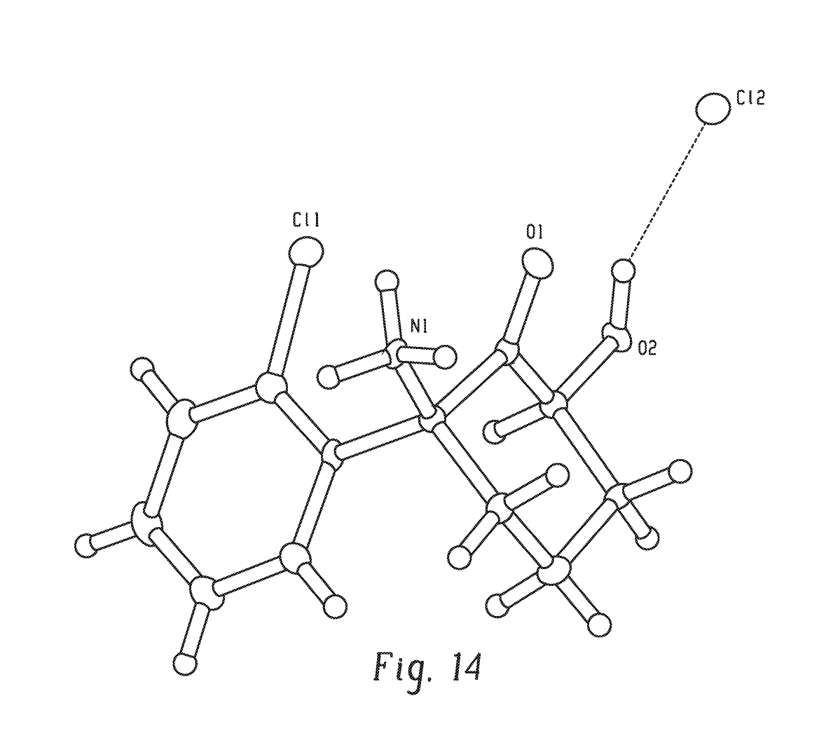

All non-hydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. All other hydrogen atoms (H-bonding) were located in the difference map. Their relative positions were restrained using DFIX commands and their thermals freely refined. The absolute stereochemistry of the molecule was established by anomalous dispersion using the Parson's method with a Flack parameter of -0.001. A depiction of the crystal structure is shown in FIG. 14. Crystallographic data are summarized in Tables 1-6.

TABLE 1
Crystal data and structure refinement for
(2S,6S)-hydroxynorketamine hydrochloride

| Property | Result | |
|---|---|---|
| Temperature | 100.0 K | |
| Wavelength | 0.71073 Å | |
| Crystal system | Monoclinic | |
| Space group | P 1 21 1 | |
| Unit cell dimensions | a = 7.3493(8) Å | α = 90°. |
| | b = 7.4846(8) Å | β = 96.866(3)°. |
| | c = 11.3404(12) Å | γ = 90°. |

| | |
|---|---|
| Volume | 619.32(12) Å³ |
| Z | 2 |
| Density (calculated) | 1.481 Mg/m³ |
| Absorption coefficient | 0.513 mm⁻¹ |
| F(000) | 288 |
| Crystal size | 0.227 × 0.215 × 0.106 mm³ |
| Crystal color, habit | Colorless Block |
| Theta range for data collection | 1.809 to 28.411° |
| Index ranges | −9 <= h <= 9, −9 <= k <= 9, |
| | −14 <= l <= 14 |
| Reflections collected | 9466 |
| Independent reflections | 2949 [R(int) = 0.0376] |
| Completeness to | 100.0% |
| theta = 25.000° | |
| Absorption correction | Semi-empirical from equivalents |
| Max. and min. transmission | 0.0962 and 0.0677 |
| Refinement method | Full-matrix least-squares on F² |
| Data/restraints/parameters | 2949/5/170 |
| Goodness-of-fit on F² | 1.075 |
| Final R indices [I > | R1 = 0.0239, wR2 = 0.0624 |
| 2sigma(I)] | |
| R indices (all data) | R1 = 0.0245, wR2 = 0.0629 |
| Absolute structure parameter | 0.00(2) |
| Extinction coefficient | n/a |
| Largest diff. peak and hole | 0.287 and −0.204 e.Å⁻³ |

TABLE 2
Atomic coordinates (×10⁴) and equivalent isotropic
displacement parameters (Å² × 10³) for (2S,6S)-
hydroxynorketamine hydrochloride. U(eq) is defined as one third
of the trace of the orthogonalized U^{ij} tensor.

| | X | y | z | U(eq) |
|---|---|---|---|---|
| Cl(1) | 6563(1) | 1930(1) | 1363(1) | 22(1) |
| O(1) | 5226(2) | 2952(2) | 3850(1) | 19(1) |
| O(2) | 1922(2) | 4022(2) | 2743(1) | 19(1) |
| N(1) | 8564(2) | 4290(2) | 3690(2) | 16(1) |
| C(1) | 5225(2) | 4235(3) | 3197(2) | 15(1) |
| C(2) | 3480(2) | 5092(2) | 2626(2) | 16(1) |
| C(3) | 3299(3) | 6901(3) | 3233(2) | 18(1) |
| C(4) | 4997(3) | 8055(3) | 3174(2) | 19(1) |
| C(5) | 6740(2) | 7066(3) | 3678(2) | 17(1) |
| C(6) | 6981(2) | 5272(3) | 3034(2) | 14(1) |
| C(7) | 7326(2) | 5480(3) | 1734(2) | 15(1) |
| C(8) | 7195(3) | 4052(3) | 939(2) | 17(1) |
| C(9) | 7583(3) | 4231(3) | −224(2) | 21(1) |
| C(10) | 8130(3) | 5875(3) | −621(2) | 24(1) |
| C(11) | 8284(3) | 7311(3) | 146(2) | 23(1) |
| C(12) | 7907(3) | 7117(3) | 1311(2) | 19(1) |
| Cl(2) | 376(1) | 481(1) | 3708(1) | 18(1) |

TABLE 3
Bond lengths [Å] and angles [°] for
(2S,6S)-hydroxynorketamine hydrochloride

| | Bond Length | | Bond Angle |
|---|---|---|---|
| Bond | (Å) | Bonds in Angle | (°) |
| Cl(1)—C(8) | 1.739(2) | C(2)—O(2)—H(2) | 113(2) |
| O(1)—C(1) | 1.213(3) | H(1A)—N(1)—H(1B) | 105(2) |
| O(2)—H(2) | 0.90(2) | H(1A)—N(1)—H(1C) | 109(2) |
| O(2)—C(2) | 1.417(2) | H(1B)—N(1)—H(1C) | 103(2) |
| N(1)—H(1A) | 0.937(19) | C(6)—N(1)—H(1A) | 110.7(17) |
| N(1)—H(1B) | 0.93(2) | C(6)—N(1)—H(1B) | 115.3(16) |
| N(1)—H(1C) | 0.94(2) | C(6)—N(1)—H(1C) | 112.4(16) |
| N(1)—C(6) | 1.496(2) | O(1)—C(1)—C(2) | 122.48(16) |
| C(1)—C(2) | 1.509(3) | O(1)—C(1)—C(6) | 122.31(18) |
| C(1)—C(6) | 1.536(2) | C(2)—C(1)—C(6) | 114.63(16) |
| C(2)—H(2A) | 1.0000 | O(2)—C(2)—C(1) | 112.02(15) |
| C(2)—C(3) | 1.532(3) | O(2)—C(2)—H(2A) | 109.1 |
| C(3)—H(3A) | 0.9900 | O(2)—C(2)—C(3) | 110.04(15) |
| C(3)—H(3B) | 0.9900 | C(1)—C(2)—H(2A) | 109.1 |
| C(3)—C(4) | 1.526(3) | C(1)—C(2)—C(3) | 107.38(16) |
| C(4)—H(4A) | 0.9900 | C(3)—C(2)—H(2A) | 109.1 |
| C(4)—H(4B) | 0.9900 | C(2)—C(3)—H(3A) | 109.3 |
| C(4)—C(5) | 1.529(3) | C(2)—C(3)—H(3B) | 109.3 |
| C(5)—H(5A) | 0.9900 | H(3A)—C(3)—H(3B) | 108.0 |
| C(5)—H(5B) | 0.9900 | C(4)—C(3)—C(2) | 111.40(15) |
| C(5)—C(6) | 1.548(3) | C(4)—C(3)—H(3A) | 109.3 |
| C(6)—C(7) | 1.534(3) | C(4)—C(3)—H(3B) | 109.3 |
| C(7)—C(8) | 1.394(3) | C(3)—C(4)—H(4B) | 109.4 |
| C(7)—C(12) | 1.401(3) | C(3)—C(4)—C(5) | 111.26(16) |
| C(8)—C(9) | 1.389(3) | H(4A)—C(4)—H(4B) | 108.0 |
| C(9)—H(9) | 0.9500 | C(5)—C(4)—H(4A) | 109.4 |
| C(9)—C(10) | 1.386(3) | C(5)—C(4)—H(4B) | 109.4 |
| C(10)—H(10) | 0.9500 | C(4)—C(5)—H(5A) | 109.1 |
| C(10)—C(11) | 1.379(3) | C(4)—C(5)—H(5B) | 109.1 |
| C(11)—H(11) | 0.9500 | C(4)—C(5)—C(6) | 112.43(16) |
| C(11)—C(12) | 1.389(3) | H(5A)—C(5)—H(5B) | 107.8 |
| C(12)—H(12) | 0.9500 | C(6)—C(5)—H(5A) | 109.1 |
| | | C(6)—C(5)—H(5B) | 109.1 |
| | | N(1)—C(6)—C(1) | 107.84(15) |
| | | N(1)—C(6)—C(5) | 108.54(15) |
| | | N(1)—C(6)—C(7) | 108.62(14) |
| | | C(1)—C(6)—C(5) | 103.68(14) |
| | | C(7)—C(6)—C(1) | 113.84(15) |
| | | C(7)—C(6)—C(5) | 114.01(16) |
| | | C(8)—C(7)—C(6) | 122.52(18) |
| | | C(8)—C(7)—C(12) | 116.72(18) |
| | | C(12)—C(7)—C(6) | 120.65(18) |
| | | C(7)—C(8)—Cl(1) | 121.42(15) |
| | | C(9)—C(8)—Cl(1) | 116.29(17) |
| | | C(9)—C(8)—C(7) | 122.29(19) |
| | | C(8)—C(9)—H(9) | 120.2 |
| | | C(10)—C(9)—C(8) | 119.6(2) |
| | | C(10)—C(9)—H(9) | 120.2 |
| | | C(9)—C(10)—H(10) | 120.3 |
| | | C(11)—C(10)—C(9) | 119.47(19) |
| | | C(11)—C(10)—H(10) | 120.3 |
| | | C(10)—C(11)—H(11) | 119.7 |
| | | C(10)—C(11)—C(12) | 120.5(2) |
| | | C(12)—C(11)—H(11) | 119.7 |
| | | C(7)—C(12)—H(12) | 119.3 |
| | | C(11)—C(12)—C(7) | 121.4(2) |
| | | C(11)—C(12)—H(12) | 119.3 |

TABLE 4
Anisotropic displacement parameters (Å² × 10³) for (2S,6S)-
hydroxynorketamine hydrochloride. The anisotropic displacement factor
exponent takes the form: −2π²[h² a*²U¹¹ + . . . + 2 h k a* b* U¹²]

| | U¹¹ | U²² | U³³ | U²³ | U¹³ | U¹² |
|---|---|---|---|---|---|---|
| Cl(1) | 27(1) | 16(1) | 22(1) | −3(1) | 3(1) | −2(1) |
| O(1) | 19(1) | 18(1) | 21(1) | 3(1) | 5(1) | 0(1) |
| O(2) | 13(1) | 20(1) | 23(1) | 2(1) | 3(1) | −1(1) |
| N(1) | 14(1) | 18(1) | 15(1) | 0(1) | 2(1) | 1(1) |
| C(1) | 16(1) | 15(1) | 14(1) | −4(1) | 4(1) | 1(1) |
| C(2) | 14(1) | 16(1) | 18(1) | 1(1) | 3(1) | −1(1) |
| C(3) | 17(1) | 17(1) | 21(1) | −2(1) | 3(1) | 4(1) |
| C(4) | 20(1) | 15(1) | 22(1) | −1(1) | 2(1) | 1(1) |
| C(5) | 18(1) | 15(1) | 18(1) | −2(1) | 1(1) | 1(1) |
| C(6) | 13(1) | 14(1) | 15(1) | −1(1) | 2(1) | 1(1) |
| C(7) | 12(1) | 18(1) | 16(1) | 2(1) | 1(1) | 2(1) |
| C(8) | 15(1) | 18(1) | 18(1) | 1(1) | 1(1) | 1(1) |
| C(9) | 19(1) | 28(1) | 16(1) | −2(1) | 1(1) | 4(1) |
| C(10) | 21(1) | 35(1) | 17(1) | 7(1) | 3(1) | 5(1) |
| C(11) | 18(1) | 27(1) | 24(1) | 8(1) | 4(1) | 1(1) |
| C(12) | 16(1) | 20(1) | 21(1) | 2(1) | 2(1) | −2(1) |
| Cl(2) | 20(1) | 16(1) | 18(1) | 0(1) | 1(1) | 1(1) |

TABLE 5
Hydrogen coordinates (×10⁴) and isotropic
displacement parameters (Å² × 10³) for
(2S,6S)-hydroxynorketamine hydrochloride.

| | x | y | z | U(eq) |
|---|---|---|---|---|
| H(2) | 2200(40) | 3010(30) | 3160(30) | 40(9) |
| H(1A) | 9650(30) | 4530(40) | 3360(20) | 23(6) |
| H(1B) | 8460(30) | 3060(30) | 3690(20) | 19(6) |
| H(1C) | 8730(40) | 4570(40) | 4506(19) | 23(6) |
| H(2A) | 3575 | 5291 | 1764 | 19 |
| H(3A) | 2209 | 7535 | 2840 | 22 |
| H(3B) | 3116 | 6706 | 4074 | 22 |
| H(4A) | 4882 | 9168 | 3631 | 23 |
| H(4B) | 5086 | 8387 | 2338 | 23 |
| H(5A) | 6695 | 6831 | 4533 | 20 |
| H(5B) | 7815 | 7836 | 3604 | 20 |
| H(9) | 7474 | 3232 | −745 | 25 |
| H(10) | 8397 | 6012 | −1416 | 29 |
| H(11) | 8650 | 8442 | −124 | 27 |
| H(12) | 8047 | 8115 | 1832 | 23 |

TABLE 6
Hydrogen bonds for (2S,6S)-hydroxynorketamine
hydrochloride 3 [Å and °].

| D-H . . . A | d(D-H) | d(H . . . A) | d(D . . . A) | <(DHA) |
|---|---|---|---|---|
| O(2)—H(2) . . . | 0.90(2) | 2.44(3) | 3.1317(16) | 133(3) |
| Cl(2) | | | | |
| N(1)—H(1A) . . . | 0.937(19) | 1.92(2) | 2.814(2) | 158(2) |
| O(2)#1 | | | | |
| N(1)—H(1B) . . . | 0.93(2) | 2.39(2) | 3.1460(19) | 139(2) |
| Cl(2)#1 | | | | |
| N(1)—H(1C) . . . | 0.94(2) | 2.16(2) | 3.0925(18) | 168(2) |
| Cl(2)#2 | | | | |

Symmetry transformations used to generate equivalent atoms:
#1 x + 1, y, z
#2 −x + 1, y + 1/2, −z + 1

### X-Ray Crystallography of (2R,6R)-Hydroxynorketamine Hydrochloride

The single crystal X-ray diffraction studies were carried out on a Bruker Kappa APEX-II CCD diffractometer equipped with Mo K_{α} radiation (λ=0.71073 Å). Crystals of the subject compound were grown by slow evaporation of an isopropanol solution. A 0.157×0.131×0.098 mm piece of a colorless block was mounted on a Cryoloop with Paratone oil. Data were collected in a nitrogen gas stream at 100(2) K using A 0.157×0.131×0.098 mm piece of a colorless block was mounted on a Cryoloop with Paratone oil. Data were collected in a nitrogen gas stream at 100(2) K using ϕ and ω̅ scans. Crystal-to-detector distance was 40 mm and exposure time was 3 seconds per frame using a scan width of 2.0°. Data collection was 100% complete to 25.00° in θ. A total of 7618 reflections were collected covering the indices, -9<=h<=9, -9<=k<=9, -14<=1<=14. 2927 reflections were found to be symmetry independent, with a Rᵢₙₜ of 0.0350. Indexing and unit cell refinement indicated a primitive, monoclinic lattice. The space group was found to be P2₁. The data were integrated using the Bruker SAINT software program and scaled using the SADABS software program. Solution by direct methods (SHELXT) produced a complete phasing model consistent with the proposed structure.

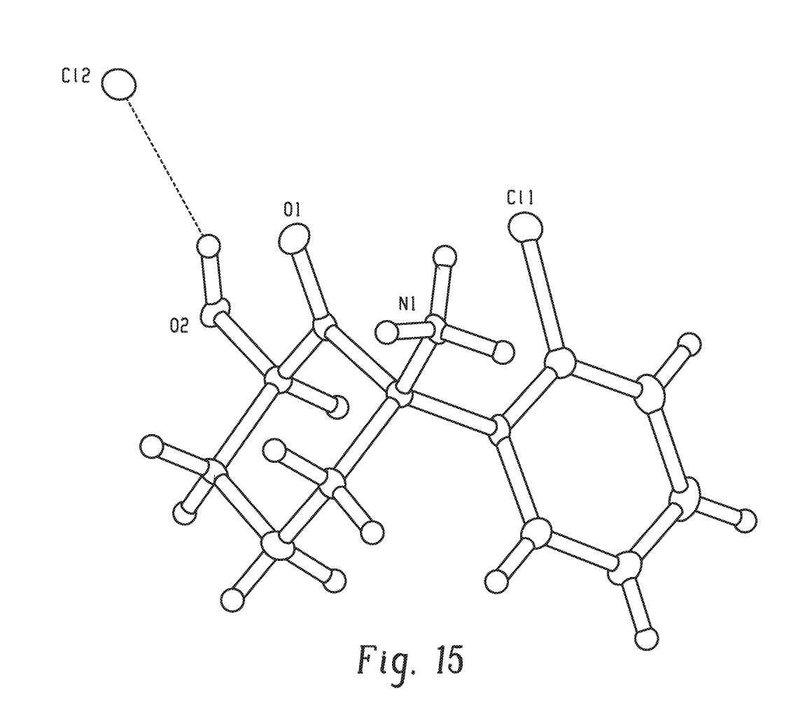

All non-hydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. All other hydrogen atoms (H-bonding) were located in the difference map. Their relative positions were restrained using DFIX commands and their thermals freely refined. The absolute stereochemistry of the molecule was established by anomalous dispersion using the Parson's method with a Flack parameter of 0.023(32). A depiction of the crystal structure is shown in FIG. 15. Crystallographic data are summarized in Tables 7-12.

TABLE 7
Crystal data and structure refinement for
(2R,6R)-hydroxynorketamine hydrochloride

| Property | Result | |
|---|---|---|
| Temperature | 100.0 K | |
| Wavelength | 0.71073 Å | |
| Crystal system | Monoclinic | |
| Space group | P 1 21 1 | |
| Unit cell dimensions | a = 7.3549(6) Å | α = 90°. |
| | b = 7.4932(5) Å | β = 96.868(2)°. |
| | c = 11.3404(12) Å | γ = 90°. |

| | |
|---|---|
| Volume | 621.02(8) Å³ |
| Z | 2 |
| Density (calculated) | 1.477 Mg/m³ |
| Absorption coefficient | 0.511 mm⁻¹ |
| F(000) | 288 |
| Crystal size | 0.157 × 0.131 × 0.098 mm³ |
| Crystal color, habit | Colorless Block |
| Theta range for data collection | 1.807 to 28.290° |
| Index ranges | −9 <= h <= 9, −9 <= k <= 9, |
| | −14 <= l <= 14 |
| Reflections collected | 7618 |
| Independent reflections | 2927 [R(int) = 0.0350] |
| Completeness to | 100.0% |
| theta = 25.000° | |
| Absorption correction | Semi-empirical from equivalents |
| Max. and min. transmission | 0.0962 and 0.0687 |
| Refinement method | Full-matrix least-squares on F² |
| Data/restraints/parameters | 2927/5/170 |
| Goodness-of-fit on F² | 1.040 |
| Final R indices [I > | R1 = 0.0265, wR2 = 0.0659 |
| 2sigma(I)] | |
| R indices (all data) | R1 = 0.0280, wR2 = 0.0669 |
| Absolute structure parameter | 0.02(3) |
| Extinction coefficient | n/a |
| Largest diff. peak and hole | 0.283 and −0.201 e.Å⁻³ |

TABLE 8
Atomic coordinates (×10⁴) and equivalent isotropic
displacement parameters (Å² × 10³) for (2R,6R)-
hydroxynorketamine hydrochloride. U(eq) is defined as one third
of the trace of the orthogonalized U^{ij} tensor.

| | X | y | z | U(eq) |
|---|---|---|---|---|
| Cl(1) | 3437(1) | 8068(1) | 8636(1) | 20(1) |
| O1) | 4777(2) | 7045(2) | 6149(1) | 18(1) |
| O(2) | 8078(2) | 5975(2) | 7255(2) | 18(1) |
| N(1) | 1437(2) | 5707(3) | 6311(2) | 14(1) |
| C(1) | 4777(3) | 5763(3) | 6802(2) | 13(1) |
| C(2) | 6518(3) | 4905(3) | 7374(2) | 14(1) |
| C(3) | 6698(3) | 3100(4) | 6768(2) | 16(1) |
| C(4) | 5001(3) | 1942(3) | 6824(2) | 17(1) |
| C(5) | 3260(3) | 2934(3) | 6323(2) | 16(1) |
| C(6) | 3023(3) | 4721(3) | 6968(2) | 13(1) |
| C(7) | 2670(3) | 4523(3) | 8268(2) | 14(1) |
| C(8) | 2804(3) | 5944(3) | 9065(2) | 16(1) |
| C(9) | 2415(3) | 5767(4) | 10223(2) | 20(1) |
| C(10) | 1875(3) | 4126(4) | 10622(2) | 23(1) |
| C(11) | 1718(3) | 2687(3) | 9853(2) | 21(1) |
| C(12) | 2095(3) | 2883(4) | 8689(2) | 18(1) |
| Cl(2) | 9623(1) | 9516(1) | 6291(1) | 17(1) |

TABLE 9
Bond lengths [Å] and angles [°] for
(2R,6R)-hydroxynorketamine hydrochloride

| | Bond Length | | Bond Angle |
|---|---|---|---|
| Bond | (Å) | Bonds in Angle | (°) |
| Cl(1)—C(8) | 1.743(2) | C(2)—O(2)—H(2) | 114(2) |
| O(1)—C(1) | 1.214(3) | H(1A)—N(1)—H(1B) | 105(3) |
| O(2)—H(2) | 0.90(2) | H(1A)—N(1)—H(1C) | 105(3) |
| O(2)—C(2) | 1.419(3) | H(1B)—N(1)—H(1C) | 109(3) |
| N(1)—H(1A) | 0.92(2) | C(6)—N(1)—H(1A) | 115.0(18) |
| N(1)—H(1B) | 0.94(2) | C(6)—N(1)—H(1B) | 111.9(18) |
| N(1)—H(1C) | 0.95(2) | C(6)—N(1)—H(1C) | 110.2(17) |
| N(1)—C(6) | 1.502(3) | O(1)—C(1)—C(2) | 122.56(19) |
| C(1)—C(2) | 1.508(3) | O(1)—C(1)—C(6) | 122.52(19) |
| C(1)—C(6) | 1.539(3) | C(2)—C(1)—C(6) | 114.35(19) |
| C(2)—H(2A) | 1.0000 | O(2)—C(2)—C(1) | 111.90(18) |
| C(2)—C(3) | 1.530(3) | O(2)—C(2)—H(2A) | 109.2 |
| C(3)—H(3A) | 0.9900 | O(2)—C(2)—C(3) | 109.99(17) |
| C(3)—H(3B) | 0.9900 | C(1)—C(2)—H(2A) | 109.2 |
| C(3)—C(4) | 1.528(3) | C(1)—C(2)—C(3) | 107.32(18) |
| C(4)—H(4A) | 0.9900 | C(3)—C(2)—H(2A) | 109.2 |
| C(4)—H(4B) | 0.9900 | C(2)—C(3)—H(3A) | 109.3 |
| C(4)—C(5) | 1.531(3) | C(2)—C(3)—H(3B) | 109.3 |
| C(5)—H(5A) | 0.9900 | H(3A)—C(3)—H(3B) | 108.0 |
| C(5)—H(5B) | 0.9900 | C(4)—C(3)—C(2) | 111.61(18) |
| C(5)—C(6) | 1.546(3) | C(4)—C(3)—H(3A) | 109.3 |
| C(6)—C(7) | 1.535(3) | C(4)—C(3)—H(3B) | 109.3 |
| C(7)—C(8) | 1.393(3) | C(3)—C(4)—H(4A) | 109.4 |
| C(7)—C(12) | 1.401(3) | C(3)—C(4)—H(4B) | 109.4 |
| C(8)—C(9) | 1.385(3) | C(3)—C(4)—C(5) | 111.11(19) |
| C(9)—H(9) | 0.9500 | H(4A)—C(4)—H(4B) | 108.0 |
| C(9)—C(10) | 1.385(4) | C(5)—C(4)—H(4A) | 109.4 |
| C(10)—H(10) | 0.9500 | C(5)—C(4)—H(4B) | 109.4 |
| C(10)—C(11) | 1.383(4) | C(4)—C(5)—H(5A) | 109.1 |
| C(11)—H(11) | 0.9500 | C(4)—C(5)—H(5B) | 109.1 |
| C(11)—C(12) | 1.390(3) | C(4)—C(5)—C(6) | 112.40(18) |
| C(12)—H(12) | 0.9500 | H(5A)—C(5)—H(5B) | 107.9 |
| | | C(6)—C(5)—H(5A) | 109.1 |
| | | C(6)—C(5)—H(5B) | 109.1 |
| | | N(1)—C(6)—C(1) | 107.57(18) |
| | | N(1)—C(6)—C(5) | 108.39(17) |
| | | N(1)—C(6)—C(7) | 108.37(17) |
| | | C(1)—C(6)—C(5) | 103.73(16) |
| | | C(7)—C(6)—C(1) | 114.02(17) |
| | | C(7)—C(6)—C(5) | 114.42(19) |
| | | C(8)—C(7)—C(6) | 122.9(2) |
| | | C(8)—C(7)—C(12) | 116.8(2) |
| | | C(12)—C(7)—C(6) | 120.3(2) |
| | | C(7)—C(8)—Cl(1) | 121.18(17) |
| | | C(9)—C(8)—Cl(1) | 116.4(2) |
| | | C(9)—C(8)—C(7) | 122.4(2) |
| | | C(8)—C(9)—H(9) | 120.1 |
| | | C(8)—C(9)—C(10) | 119.7(2) |
| | | C(10)—C(9)—H(9) | 120.1 |
| | | C(9)—C(10)—H(10) | 120.3 |
| | | C(11)—C(10)—C(9) | 119.4(2) |
| | | C(11)—C(10)—H(10) | 120.3 |
| | | C(10)—C(11)—H(11) | 119.8 |
| | | C(10)—C(11)—C(12) | 120.4(2) |
| | | C(12)—C(11)—H(11) | 119.8 |
| | | C(7)—C(12)—H(12) | 119.4 |
| | | C(11)—C(12)—C(7) | 121.3(2) |
| | | C(11)—C(12)—H(12) | 119.4 |

TABLE 10
Anisotropic displacement parameters (Å² × 10³) for (2R,6R)-
hydroxynorketamine hydrochloride. The anisotropic displacement factor
exponent takes the form: −2π²[h² a*²U¹¹ + . . . + 2 h k a* b* U¹²]

| | U¹¹ | U²² | U³³ | U²³ | U¹³ | U¹² |
|---|---|---|---|---|---|---|
| Cl(1) | 26(1) | 15(1) | 20(1) | −3(1) | 3(1) | −2(1) |
| O(1) | 18(1) | 17(1) | 19(1) | 4(1) | 5(1) | 0(1) |
| O(2) | 12(1) | 19(1) | 22(1) | 3(1) | 2(1) | −1(1) |
| N(1) | 13(1) | 16(1) | 14(1) | −1(1) | 2(1) | 1(1) |
| C(1) | 13(1) | 14(1) | 13(1) | −3(1) | 4(1) | 0(1) |
| C(2) | 13(1) | 15(1) | 16(1) | 1(1) | 2(1) | −1(1) |
| C(3) | 15(1) | 15(1) | 19(1) | −1(1) | 2(1) | 5(1) |
| C(4) | 18(1) | 12(1) | 21(1) | −2(1) | 1(1) | 1(1) |
| C(5) | 16(1) | 16(1) | 16(1) | −3(1) | 1(1) | 0(1) |
| C(6) | 11(1) | 14(1) | 14(1) | 0(1) | 1(1) | 1(1) |
| C(7) | 12(1) | 18(1) | 14(1) | 2(1) | 1(1) | 1(1) |
| C(8) | 14(1) | 18(1) | 18(1) | 2(1) | 1(1) | 1(1) |
| C(9) | 18(1) | 26(1) | 16(1) | −2(1) | 1(1) | 4(1) |
| C(10) | 18(1) | 34(2) | 16(1) | 6(1) | 4(1) | 3(1) |
| C(11) | 17(1) | 24(1) | 23(1) | 8(1) | 2(1) | 0(1) |
| C(12) | 15(1) | 20(1) | 19(1) | 1(1) | 2(1) | −2(1) |
| Cl(2) | 19(1) | 15(1) | 16(1) | 1(1) | 1(1) | 1(1) |

TABLE 11
Hydrogen coordinates (×10⁴) and isotropic
displacement parameters (Å² × 10³) for
(2R,6R)-hydroxynorketamine hydrochloride.

| | x | y | z | U(eq) |
|---|---|---|---|---|
| H(2) | 7830(50) | 7000(40) | 6860(30) | 41(10) |
| H(1A) | 1540(40) | 6930(30) | 6330(20) | 22(8) |
| H(1B) | 1270(40) | 5410(40) | 5500(20) | 23(7) |
| H(1C) | 340(30) | 5450(40) | 6650(20) | 20(7) |
| H(2A) | 6423 | 4708 | 8236 | 17 |
| H(3A) | 6881 | 3297 | 5928 | 20 |
| H(3B) | 7788 | 2467 | 7160 | 20 |
| H(4A) | 4913 | 1604 | 7659 | 21 |
| H(4B) | 5117 | 834 | 6364 | 21 |
| H(5A) | 2184 | 2166 | 6396 | 19 |
| H(5B) | 3304 | 3172 | 5468 | 19 |
| H(9) | 2518 | 6766 | 10741 | 24 |
| H(10) | 1614 | 3989 | 11417 | 27 |
| H(11) | 1351 | 1557 | 10123 | 26 |
| H(12) | 1960 | 1887 | 8168 | 21 |

TABLE 12
Hydrogen bonds for (2R,6R)-hydroxynorketamine
hydrochloride [Å and °].

| D-H . . . A | d(D-H) | d(H . . . A) | d(D . . . A) | <(DHA) |
|---|---|---|---|---|
| O(2)—H(2) . . . | 0.90(2) | 2.43(3) | 3.1348(18) | 135(3) |
| Cl(2) | | | | |
| N(1)—H(1A) . . . | 0.92(2) | 2.39(3) | 3.149(2) | 140(2) |
| Cl(2)#1 | | | | |
| N(1)—H(1B) . . . | 0.94(2) | 2.16(2) | 3.095(2) | 169(2) |
| Cl(2)#2 | | | | |
| N(1)—H(1C) . . . | 0.95(2) | 1.92(2) | 2.816(2) | 156(3) |
| O(2)#1 | | | | |

Symmetry transformations used to generate equivalent atoms:
#1 x + 1, y, z
#2 −x + 1, y + 1/2, −z + 1

### Pharmaceutical Compositions

Compounds disclosed herein can be administered as the neat chemical, but are preferably administered as a pharmaceutical composition. Accordingly, the disclosure provides pharmaceutical compositions comprising a (2S,6S)-HNK, (2R,6R)-HNK, or a salt, hydrate, or prodrug thereof, together with at least one pharmaceutically acceptable carrier. The pharmaceutical composition may contain (2S,6S)-HNK, (2R,6R)-HNK, or a salt, hydrate, or prodrug thereof as the only active agent, but may contain one or more additional active agents. In certain embodiments the pharmaceutical composition is an oral dosage form that contains from about 1 mg to about 5000 mg, from about 10 mg to about 1000 mg, or from about 50 mg to about 500 mg of an active agent which is purified (2R,6R)-hydroxynorketamine, purified (2S,6S)-hydroxynorketamine, or a combination thereof, and optionally from about 0.1 mg to about 2000 mg, from about 10 mg to about 1000 mg, from about 100 mg to about 800 mg, or from about 200 mg to about 600 mg of an additional active agent in a unit dosage form.

Compounds disclosed herein may be administered orally, topically, parenterally, by inhalation or nasal spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions can be formulated for oral administration. Preferred oral dosage forms are formulated for once a day or twice a day administration. These compositions contain between 0.1 and 99 weight % (wt. %) of (2S,6S)-HNK, (2R,6R)-HNK, or a salt, hydrate, or prodrug thereof. Some embodiments contain from about 25 wt. % to about 50 wt. % or from about 5 wt. % to about 75 wt. % of (2S,6S)-HNK, (2R,6R)-HNK, or a salt, hydrate, or prodrug thereof.

### Methods of Treatment

Methods of treatment include providing certain dosage amounts of (2S,6S)-HNK, (2R,6R)-HNK, or a salt, hydrate, or prodrug thereof to a patient. Dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration.

In certain embodiments a therapeutically effect amount is an amount that provide a plasma Cₘₐₓ of (2S,6S)-HNK, (2R,6R)-HNK, or a salt, hydrate, or prodrug thereof of about of 0.25 mcg/mL to about 125 mcg/mL, or about 1 mcg/mL to about 50 mcg/mL. The disclosure also includes intravenous pharmaceutical compositions that provide about 0.2 mg to about 500 mg per dose of (2S,6S)-HNK, (2R,6R)-HNK, or a salt, hydrate, or prodrug thereof, for peripheral indications compounds that provide about 0.5 mg to about 500 mg/dose are preferred.

Methods of treatment include combination methods in which (2S,6S)-HNK, (2R,6R)-HNK, or a salt, hydrate, or prodrug thereof is administered together with an additional active agent or another therapy. Combination administration includes simultaneous administration, concurrent administration, and sequential administration where the order of administration of the additional active agent or other therapy may be before or after administration of the HNK.

Methods of treatment include methods in which the (2S,6S)-HNK, (2R,6R)-HNK, or a salt, hydrate, or prodrug thereof is administered in conjunction with psychotherapy, cognitive behavioral therapy, exposure therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy, cognitive remediation, exercise, or other types of therapy.

Methods of treatment include methods in which the (2S,6S)-HNK, (2R,6R)-HNK, or a salt, hydrate, or prodrug thereof is administered in conjunction with the use of Electroconvulsive therapy, transcranial magnetic stimulation, deep brain stimulation, use of neuromodulation devices, or other neuromodulatory therapy.

The (2S,6S)-HNK, (2R,6R)-HNK, or a salt, hydrate, or prodrug thereof may be the only active agent administered or may be administered together with an additional active agent. For example the HNK active agent may administered together with another active agent that is chosen from any of the following CNS active agents: d-cycloserine, dextromethorphan, escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, vortioxetine, vilazadone, milnacipran, levomilacipran, pramipexole, buspirone, lithium, thyroid or other type of hormones (e.g., estrogen, progesterone, testosterone), aripiprazole, brexpiprazole, cariprazine, clozapine, loxapine, lurasidone, olanzapine, paliperidone, quetiapine, risperidone, ziprasidone, carbamazepine, oxcarbazepine, gabapentin, lamotrigine, phenytoin, pregabalin, donepezil, galantamine, memantine, minocycline, rivastigmine, riluzole, tramiprosate, ketamine, or pharmaceutically active salts or prodrugs thereof, or a combination of the foregoing.

The preceding list of additional active agents is meant to be exemplary rather than fully inclusive. Additional active agents not included in the above list may be administered in combination with (2S,6S)-HNK, (2R,6R)-HNK, or a salt, hydrate, or prodrug thereof. The additional active agent will be dosed according to its approved prescribing information, though in some embodiments the additional active agent will be dosed at less the typically prescribed dose and in some instances less than the minimum approved dose.

The disclosure includes a method of treating depressive disorders where an effective amount of the compound is an amount effective to decrease depressive symptoms, wherein a decrease in depressive symptoms is the achievement of a 50% or greater reduction of symptoms identified on a depression symptom rating scale, or a score less than or equal to 7 on the HRSD₁₇, or less than or equal to 5 on the QID-SR₁₆, or less than or equal to 10 on the MADRS. Likewise the disclosure also provides a method of treating anxiety disorders, anhedonia, fatigue, and suicidal ideation comprising administering and effective amount of a compound of the disclosure, wherein an effective amount of the compound is an amount sufficient to decrease anxiety disorder symptoms, or an amount sufficient to effect an clinically significant decrease of the anxiety disorder, anhedonia, or suicidal ideation symptoms on a symptom rating scale for anxiety, anhedonia, fatigue, or suicidal ideation.

### Examples

### General Methods

### Drugs

(R,S)-ketamine, (S)-ketamine, desipramine, MK-801, phencyclidine (PCP) (Sigma-Aldrich, St. Louis, Mo., USA), (R)-ketamine (Cayman Chemicals, Ann Arbor, Mich., USA) and NBQX (National Institute of Mental Health Chemical Synthesis and Drug Supply Program) were dissolved in 0.9% saline. (2S,6S)-HNK and (2R,6R)-HNK were synthesized as described in the Examples. (2S,6S)-HNK, (2R,6R)-HNK, and 6,6-dideuteroketamine hydrochloride were synthesized and characterized both internally at the National Center for Advancing Translational Sciences and at SRI International (Menlo Park, Calif., USA) as described in this disclosure. Absolute and relative stereochemistry for (2S,6S)-HNK and (2R,6R)-HNK were confirmed by small molecule x-ray crystallography, as described in this disclosure.

All drugs were dissolved in 0.9% saline, and administered intraperitoneally (i.p.) in a volume of 7.5 ml/kg of body mass. Corticosterone (4-pregnen-11β, 21-diol-3, 20-dione 21-hemisuccinate; Steraloids, Newport, R.I., USA) was dissolved in tap water. For the electrophysiology recordings, test drugs were diluted in artificial cerebrospinal fluid (AC SF).

### Chemical Methods

All commercially available reagents and solvents were purchased and used without further purification. All microwave reactions were carried out in a sealed microwave vial equipped with a magnetic stir bar and heated in a Biotage Initiator Microwave Synthesizer. ¹H NMR and ¹³C NMR spectra were recorded on Varian 400 MHz or Varian 600 MHz spectrometers in CD₃OD or CDCl₃ as indicated. For spectra recorded in CD₃OD, chemical shifts are reported in ppm with CD₃OD (3.31 MHz) as reference for ¹H NMR spectra and CD₃OD (49.0 MHz) for ¹³C NMR spectra. Alternatively for spectra recorded in CDCl₃, chemical shifts are reported in ppm relative to deuterochloroform (7.26 ppm for ¹H NMR, 77.23 ppm for ¹³C NMR. The coupling constants (J value) are reported as Hertz (Hz). The splitting patterns of the peaks were described as: singlet (s); doublet (d); triplet (t); quartet (q); multiplet (m) and septet (septet). Samples were analyzed for purity on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 mL/min. High resolution mass spectrometry (HRMS) was recorded on Agilent 6210 Time-of-Flight (TOF) LC/MS system. Optical rotations were measured on a PerkinElmer model 341 polarimeter using a 10 cm cell, at 589 nM and room temperature.

Chiral analysis was carried out with an Agilent 1200 series HPLC using an analytical Chiralpak AD or OJ column (4.6 mm×250 mm; 5 μm). The mobile phase consisted of ethanol containing 0.1% diethylamine as component A and hexanes containing 0.1% diethylamine as component B. An isocratic gradient was run at 0.4 mL/min with 60% A.

### Biochemical Methods

### Mk-801 Displacement Binding

Bindings were performed as previously described. Test compounds were prepared in 50 mM Tris-HCl, by serial dilutions ranging from 0.05 nM to 50 μM. The radioligand, [3H]-MK-801 was diluted to a final concentration of 5 nM. 50 μl of the radioligand were dispensed into the wells of a 96-well plate containing 100 μl of 50 mM Tris-HCl (pH 8.0) and 50 μl of the test compound. Rat brain was homogenized in 50 volumes of ice-cold 50 mM Tris-HCl buffer with 10 mM ethylenediaminetetraacetic acid, pH 8.0 and the homogenate was centrifuged at 35,000×g for 15 min. The resulting pellet was resuspended in chilled 50 mM Tris-HCl (pH 8.0) and homogenized by several passages through a 26-gauge needle. 50 μl of the resultant supernatant was dispensed into each well (final reaction volume: 250 μl). The reactions were incubated for 1.5 hours at room temperature and shielded from light exposure, and then were harvested via rapid filtration onto Whatman GF/B glass fiber filters pre-soaked with 0.3% polyethyleneimine using a 96-well Brandel harvester. To reduce non-specific binding, four washes with 500 μl chilled Standard Binding buffer were performed. Filters were subsequently placed in 6-ml scintillation tubes and allowed to dry overnight and then scintillator was melted onto the filter mates and the radioactivity retained on the filters was counted in a MicroBeta scintillation counter. All assays were done in duplicates.

### Western Blots

To purify synaptoneurosomes, mouse prefrontal cortex or hippocampus were dissected and homogenized in Syn-PER Reagent (ThermoFisher Scientific, Waltham, Mass., USA; Cat #87793) with 1× protease and phosphatase inhibitor cocktail (ThermoFisher Scientific, Waltham, Mass., USA; Cat #78440). The homogenate was centrifuged for 10 min at 1,200×g at 4° C. The supernatant was centrifuged at 15,000×g for 20 min. After centrifugation, the supernatant the pellet (synaptosomal fraction) was re-suspended and sonicated in N-PER Neuronal Protein Extraction Reagent (ThermoFisher Scientific, Waltham, Mass., USA; Cat #87792). For total homogenous tissue lysates, mouse prefrontal cortex or hippocampus were homogenized and sonicated in N-PER Neuronal Protein Extraction Reagent with 1× protease & phosphatase inhibitor cocktail) Protein concentration was determined via the BCA protein assay kit (ThermoFisher Scientific, Waltham, Mass., USA; Cat #23227).

For western blotting, equal amount of proteins (10-40 μg as optimal for each antibody) for each sample were loaded into NuPage 4-12% Bis-Tris gel for electrophoresis. Nitrocellulose membranes with transferred proteins were blocked with 5% milk in TBST (TBS+0.1% Tween-20) for 1 hour and kept with primary antibodies overnight at 4° C. The following primary antibodies were used: phospho-eEF2 (Cell Signaling Technology, Danvers, Mass., USA; Cat #2331), total eEF2 (Cell Signaling Technology, Danvers, Mass., USA; Cat #2332), phospho-mTOR (Cell Signaling Technology, Danvers, Mass., USA; Cat #2971), total mTOR (Cell Signaling Technology, Danvers, Mass., USA; Cat #2983), GluR1 (Cell Signaling Technology, Danvers, Mass., USA; Cat #2983), GluR2 (Cell Signaling Technology, Danvers, Mass., USA; Cat #13607), BDNF (Santa Cruz Biotechnology, Dallas, Tex., USA; Cat # sc-546), and GAPDH (Abcam, Cambridge, Mass., USA; Cat # ab8245). The next day, blots were washed three times in PBST and incubated with horseradish peroxidase conjugated anti-mouse or anti-rabbit secondary antibody (1:5000 to 1:10000) for 1 hour. After final three washes with TBST, bands were detected using enhanced chemiluminescence (ECL) with the Syngene Imaging System (G:Box ChemiXX9). After imaging, the blots then were incubated in the stripping buffer (ThermoFisher Scientific, Waltham, Mass., USA; Cat #46430) for 10-15 min at room temperature followed by three time washes with TBST. The stripped blots were washed in blocking solution for 1 hour and incubated with the primary antibody directed against total levels of the respective protein or GAPDH for loading control. Densitometric analysis of phospho- and total immunoreactive bands for each protein was conducted using Syngene's GenTools software Immunoreactivity was normalized to the saline treated control group for each protein.

### Statistical Analyses

All statistical analyses were performed using Statistica software V10 (StatSoft Inc., Bedford, UK). Specific statistical tests used are reported in the Extended Data Table 1. ANOVAs were followed by a Holm-Šidák post hoc comparison, when significance was reached (i.e., p<0.05).

### Example 1. Ketamine, Ketamine Enantiomers, and Desipramine in Antidepressant Models

The antidepressant effects of ketamine and the classical tricyclic antidepressant desipramine were compared in male CD-1 mice in the forced-swim test at 1 hour (acute) and 24 hour (sustained) time points (forced swim test (FST); FIG. 1a) Administration of ketamine at the dose of 10 mg/kg resulted in acute and long-lasting dose-dependent antidepressant effects in the FST, whereas desipramine only decreased immobility time 1 hour post-injection.

To elucidate whether NMDA inhibition is the main mechanism underlying the antidepressant effects of ketamine, the effects of ketamine and the non-competitive NMDA receptor antagonist MK-801 in the FST were compared, and the antidepressant responses of both ketamine and MK-801 observed acutely. Only ketamine showed sustained effects following 24 hours (FIG. 1e). Moreover, the effects of ketamine's enantiomers (S)- and (R)-ketamine were assessed in the FST (FIG. 1g), novelty-suppressed feeding (NSF; FIG. 1e) and learned helplessness (LH; FIG. 1d) tests.

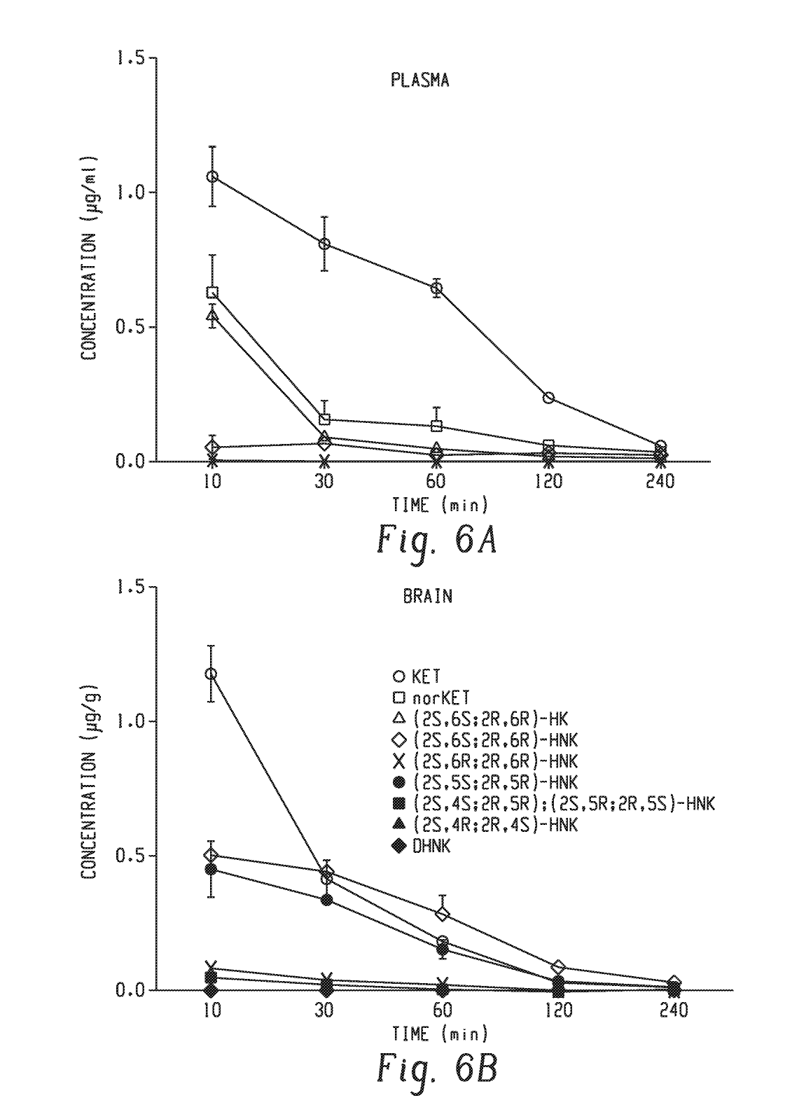

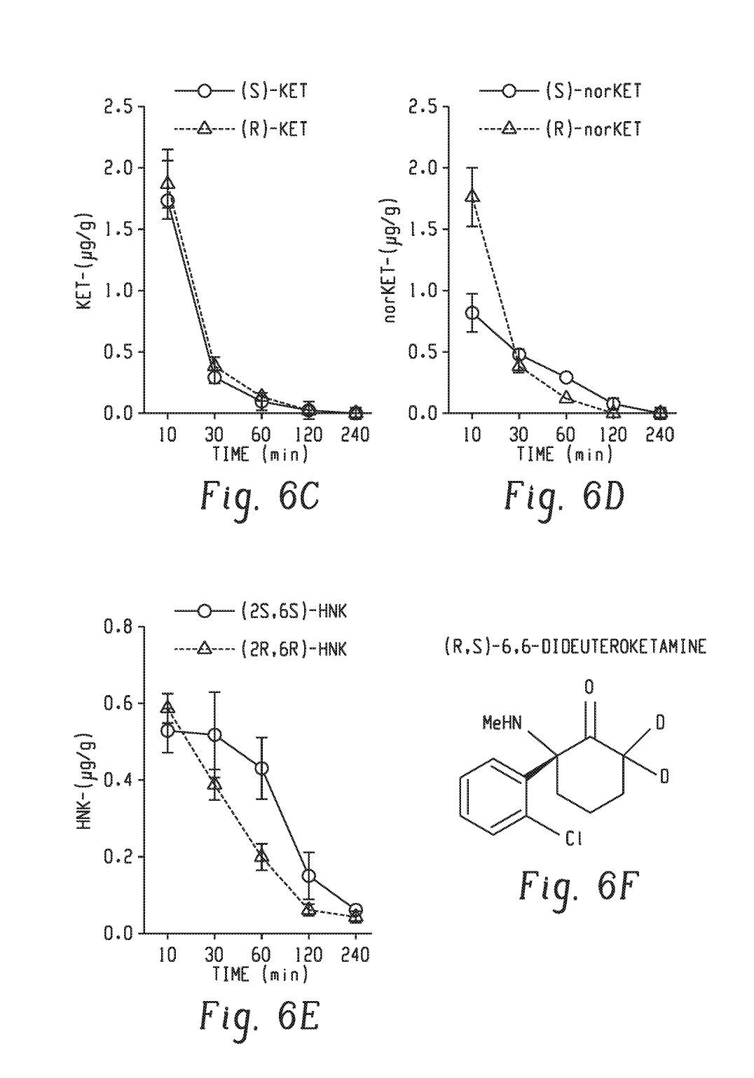

While the NMDA hypothesis of ketamine action would predict greater efficacy of (S)-ketamine since it is a ˜4 fold more potent inhibitor of the NMDA receptor than (R)-ketamine, the present results, in accordance with recent findings, demonstrate a greater potency of (R)-ketamine in all these antidepressant-predictive tasks, an effect which does not result from higher brain levels of (R)-ketamine compared to (S)-ketamine (FIG. 6c-6e). These findings indicate likely non-NMDA mechanism underlying the antidepressant responses of ketamine Determination that R-ketamine produces high brain levels establishes 2R,6R-HNK as the active metabolite.

This finding is consistent with the results of human treatment trials indicating that alternate NMDAR antagonists lack the robust, rapid, or sustained antidepressant properties of ketamine. (Newport, D J, et al., Am. J. Psychiat. (2015) 172: 950-066.) FIG. 1e shows that unlike ketamine, the NMDAR antagonist MK-801, which binds at the same receptor site as ketamine, does not exert sustained (24-hour) antidepressant-like effects in the FST, or reverse social interaction deficits induced by chronic social defeat stress (FIG. 7).

### Example 2. Metabolism of Ketamine and Locomotion Experiments

Ketamine is stereoselectively metabolized into a broad array of metabolites, including norketamine, hydroxyketamines (HK), HNK, and dehydronorketamine (DHNK) (FIG. 1f, FIG. 5). Following ketamine administration, (2S,6S;2R,6R)-HNK is the major metabolite found in the plasma and brain of mice (FIG. 6a,6b) and plasma of humans.

To directly determine if metabolism of ketamine to (2S,6S;2R,6R)-HNK is required for its antidepressant actions, ketamine was deuterated at the C6 position (6,6-dideuteroketamine) Deuteration blocks ketamine metabolism to the metabolites, 2S,6S-HNK and 2R,6R-HNK.

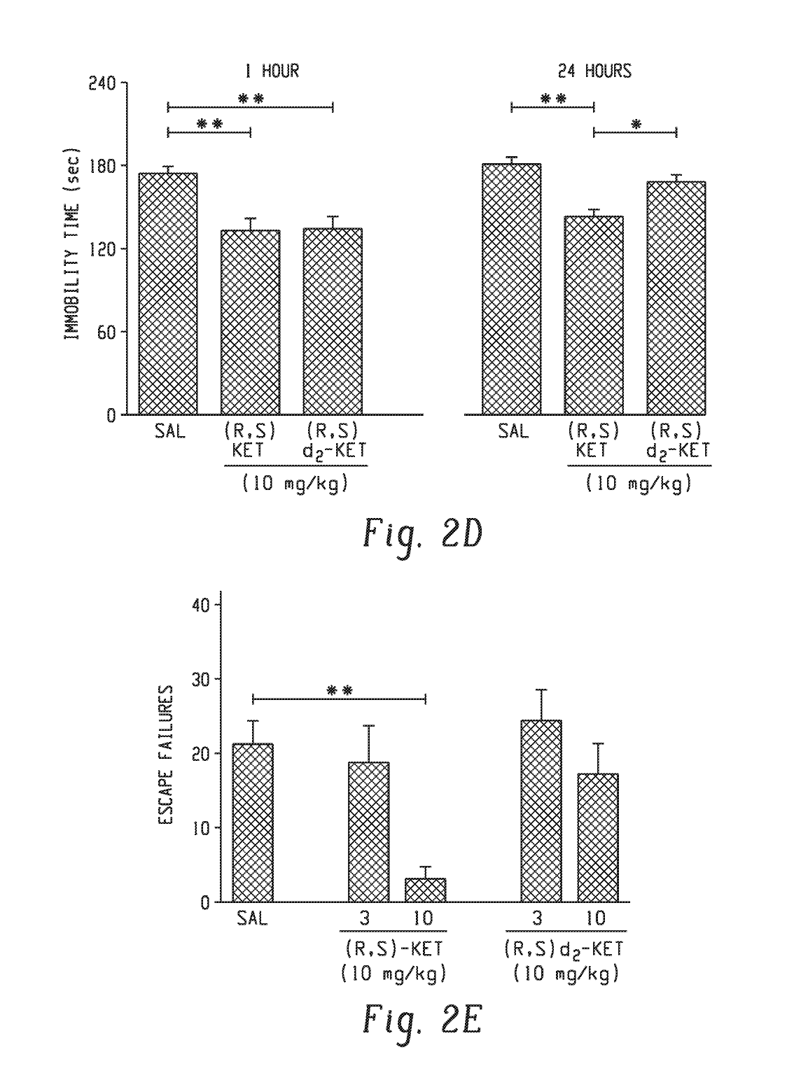

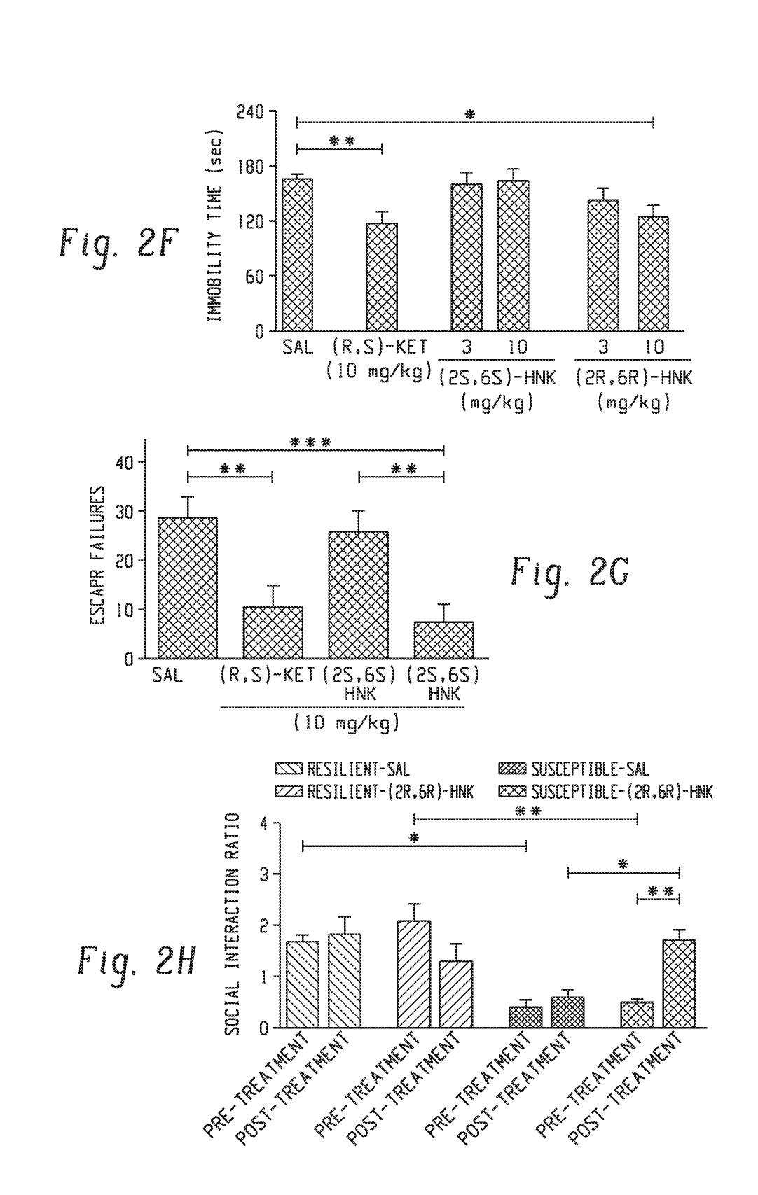

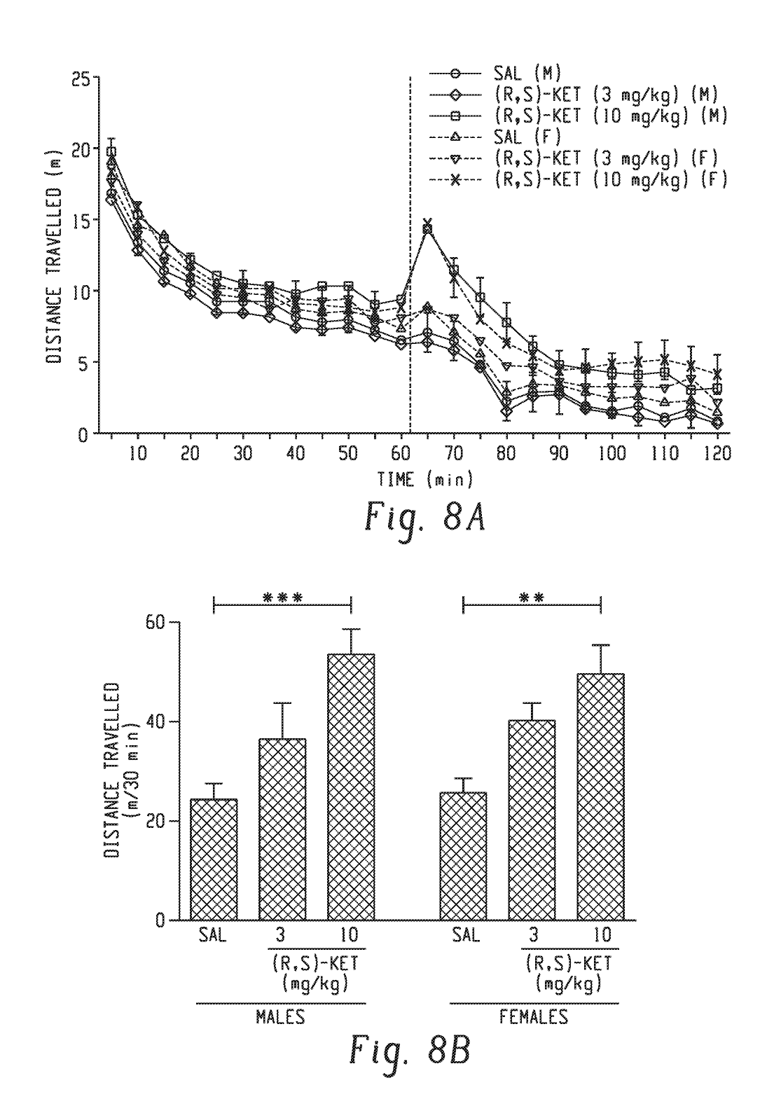

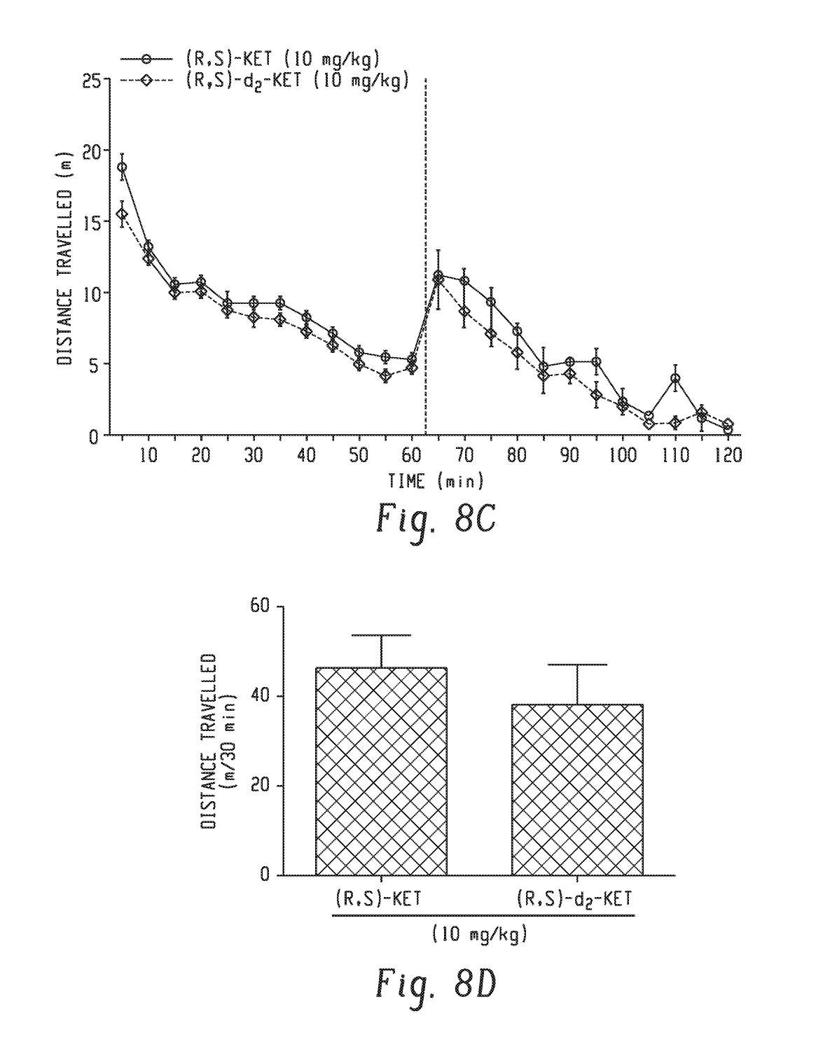

Indeed, 6,6-dideuteroketamine did not change or NMDA-mediated hyperlocomotion (FIG. 8c,8d), but robustly hindered its metabolism to (2S,6S;2R,6R)-HNK, without changes to the levels of ketamine in the brain (FIG. 2a-2c). Unlike ketamine, administration of 6,6-dideuteroketamine did not induce antidepressant actions in the FST (FIG. 2d) or LH (FIG. 2e) 24 hours after administration, indicating a critical role of (2S,6S;2R,6R)-HNK in ketamine's sustained antidepressant effects. Notably, human data reveal a positive correlation between the antidepressant responses of ketamine and plasma (2S,6S;2R,6R)-HNK metabolite levels.

### Example 3. Ketamine, Ketamine Enantiomers, and (2S,6S; 2R,6R)-HNK in Antidepressant Models

In order to investigate whether these sex-dependent antidepressant differences are explained by a different pharmacokinetic profile of ketamine in males versus females, the levels of ketamine and its metabolites were measured in the brains and plasma of mice injected with ketamine. (2S,6S;2R,6R)-HNK is the major HNK metabolite found in the plasma and brain of mice (FIG. 6a,6b), and plasma of humans FIG. 1g shows greater antidepressant potency of ketamine in female mice, similar to previous evidence revealing enhanced ketamine antidepressant responses in female rats compared to males. These differences are not associated with sex differences in ketamine-induced hyperlocomotion, which is likely mediated by NMDAR inhibition (FIG. 8a,8b).

In order to investigate whether these sex-dependent antidepressant differences are predicted by a different pharmacokinetic profile of ketamine in males versus females, the levels of ketamine and its metabolites in the brains of mice following ketamine administration were assessed. While equivalent levels of ketamine and norketamine were found, (2S,6S;2R,6R)-HNK was three fold higher in the brain of female mice compared to males (FIG. 1h-1j), suggesting a primary role of (2S,6S;2R,6R)-HNK in the antidepressant effects of ketamine. The present finding is supported by human data revealing a positive correlation between the antidepressant responses of ketamine and plasma (2S,6S;2R,6R)-HNK metabolite levels.

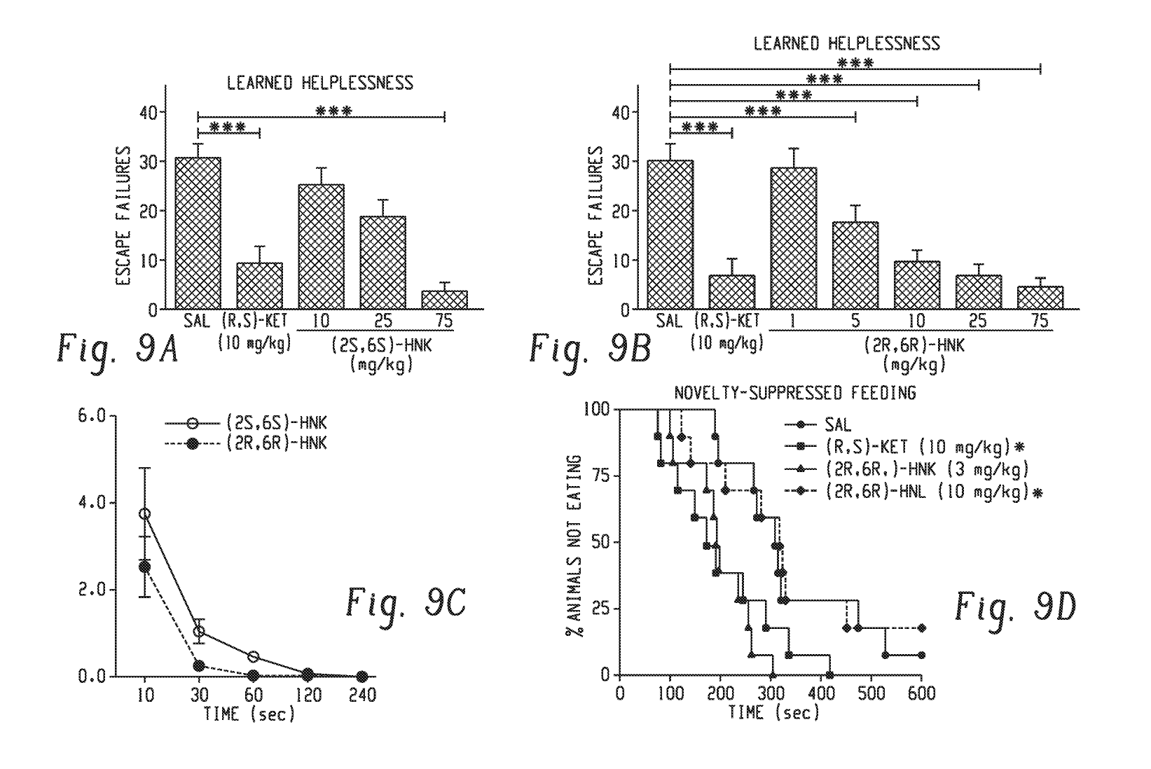

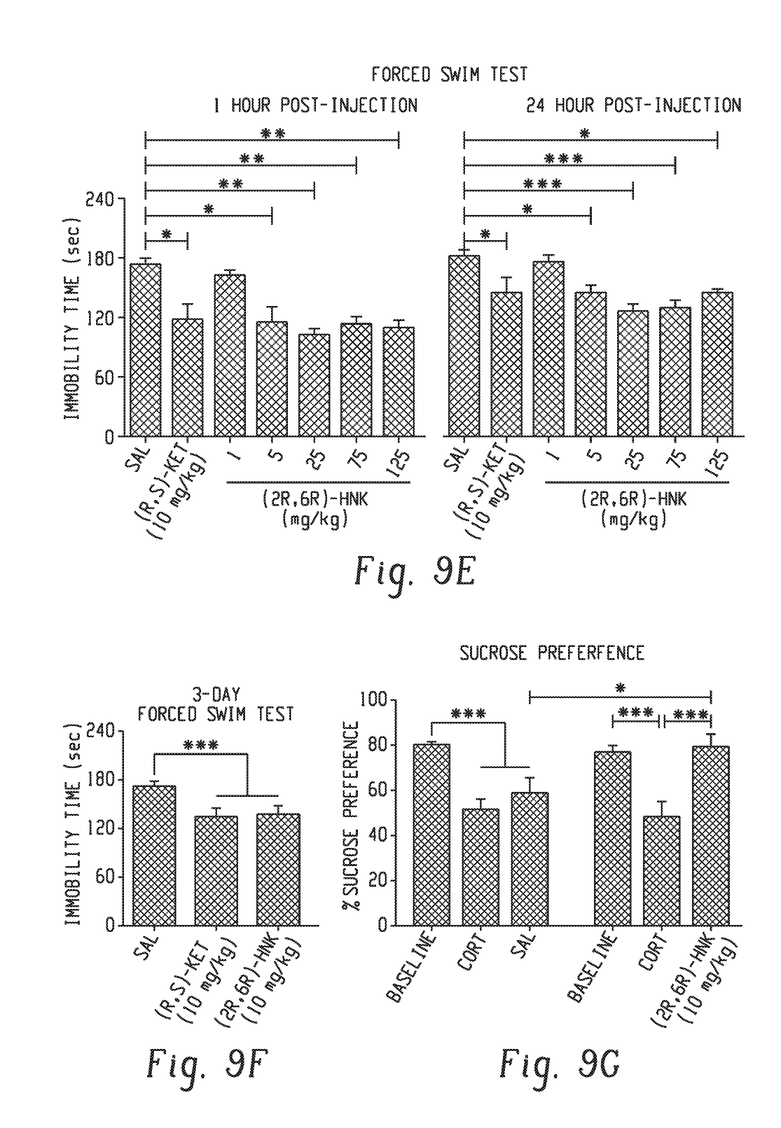

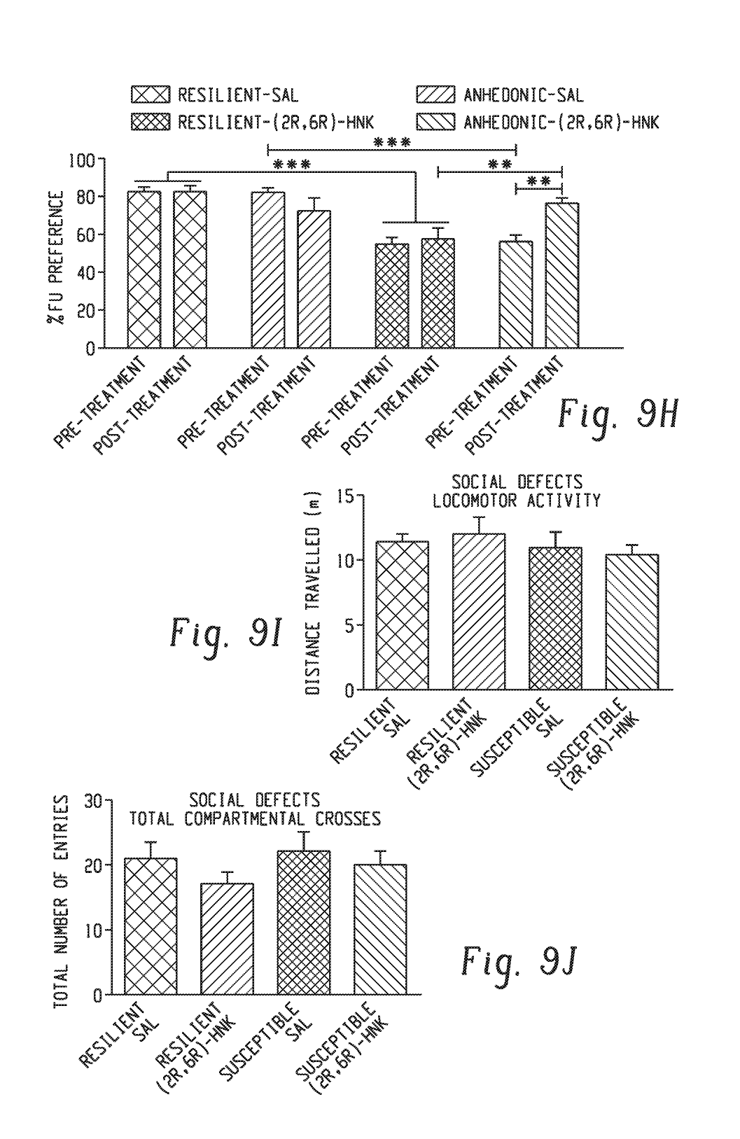

In order to directly determine whether (2S,6S)- or (2R,6R)-HNK exert ketamine-like antidepressant effects, their behavioral effects in the 24-hour FST, NSF and LH paradigms were assessed. FIG. 2f,2g and FIG. 9a,9b show more potent antidepressant effects following administration of the (2R,6R)-HNK metabolite, which is exclusively derived from (R)-ketamine, and thus consistent with the greater antidepressant actions of (R)-ketamine relative to (S)-ketamine (FIG. 1b-1d). The greater antidepressant effects of (2R,6R)-HNK do not result from higher brain levels of the drug compared to (2S,6S)-HNK (FIG. 9c). Moreover, administration of (2R,6R)-HNK resulted in a dose-dependent antidepressant action in the LH, NSF and FST tests (FIG. 9b,9d,9e). The results in the LH are important, because development of helplessness is a maladaptive response to a severe stress. This has parallels to human post traumatic stress disorder, where a similar neurobiological phenomenon is thought to occur. The results in the NSF are important, as this indicates rapid onset in an anxiety test that is only sensitive to the chronic administration of SSRIs. Similar to ketamine, a single (2R,6R)-HNK administration induced persistent antidepressant effects in the FST, lasting for at least three days (FIG. 9f). Notably, a single administration of (2R,6R)-HNK also reversed chronic corticosterone-induced anhedonia as assessed in the sucrose preference and female urine sniffing behavioral tasks (FIG. 9g,9h), as well as social avoidance induced by chronic social defeat stress (FIG. 2h; FIG. 9i-9j). These data are important, as they indicate reversal of anhedonia, potentially independent of depression such as that which occurs in schizophrenia. Furthermore, the reduction in suicidal thinking following ketamine has been linked to a reduction in anhedonia, rather than depressive symptoms per se, indicating the capacity of (2R,6R)-HNK to rapidly treat suicidal thoughts.

### Example 4. AMPA Activity

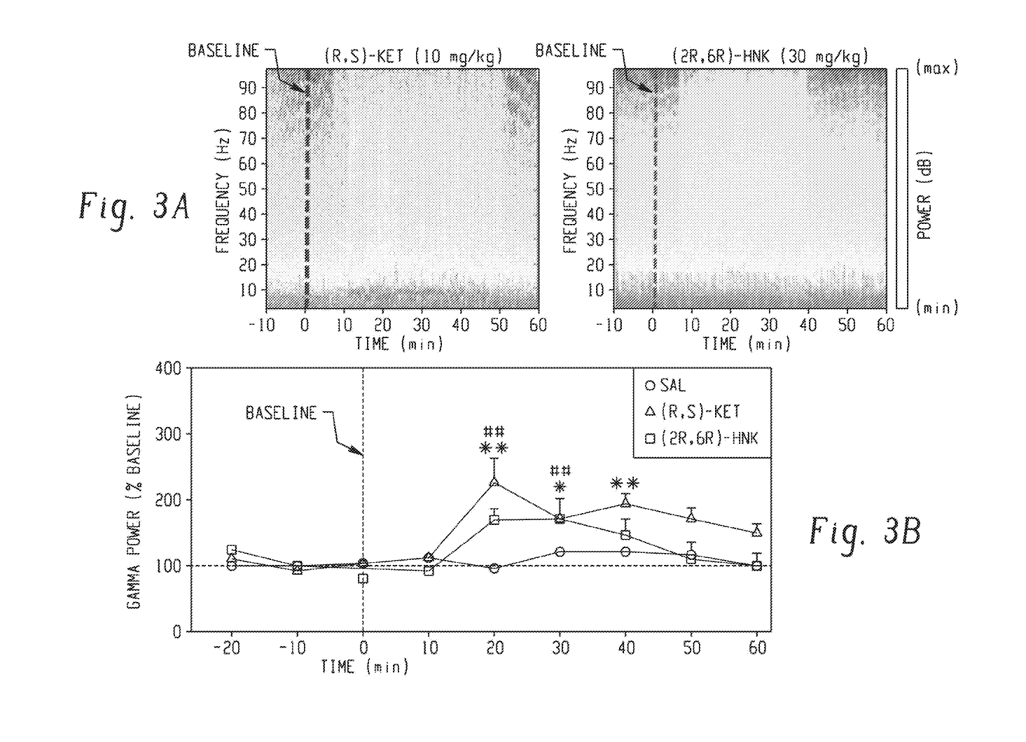

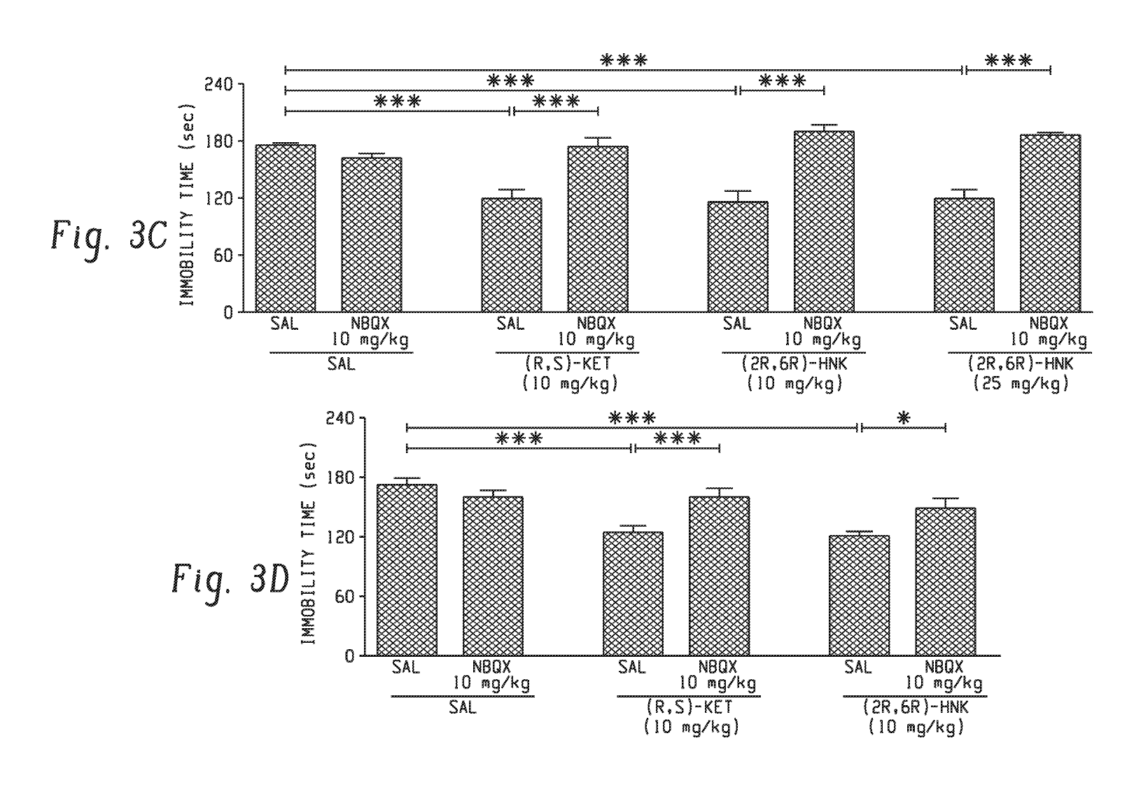

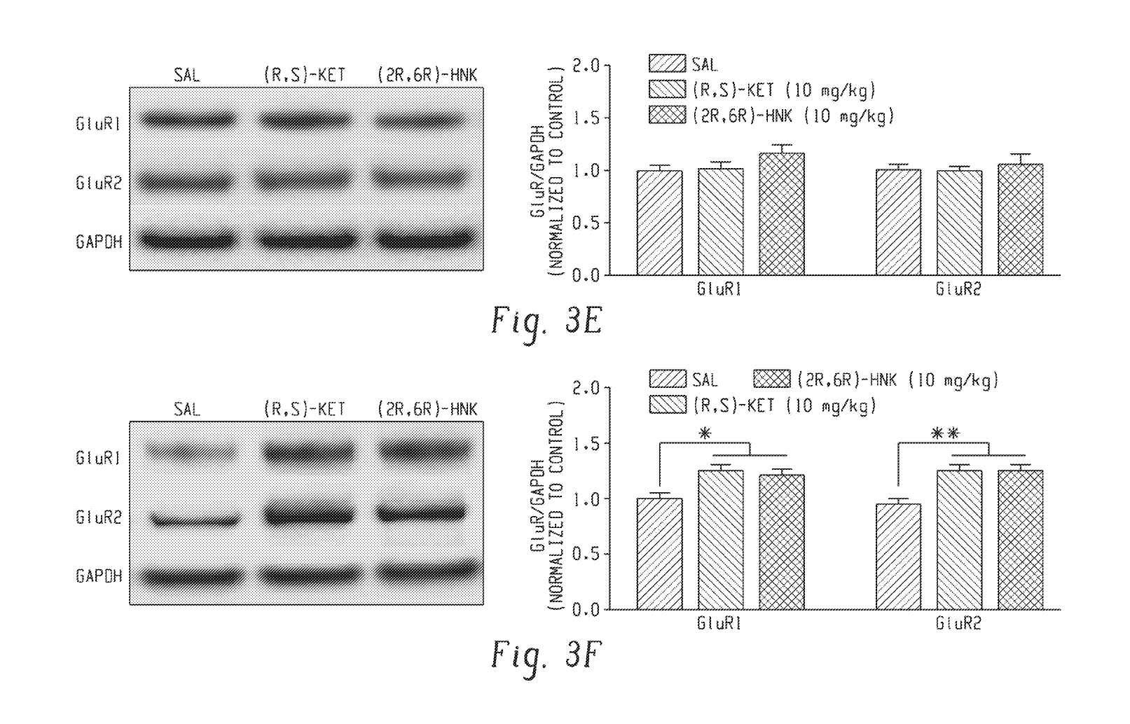

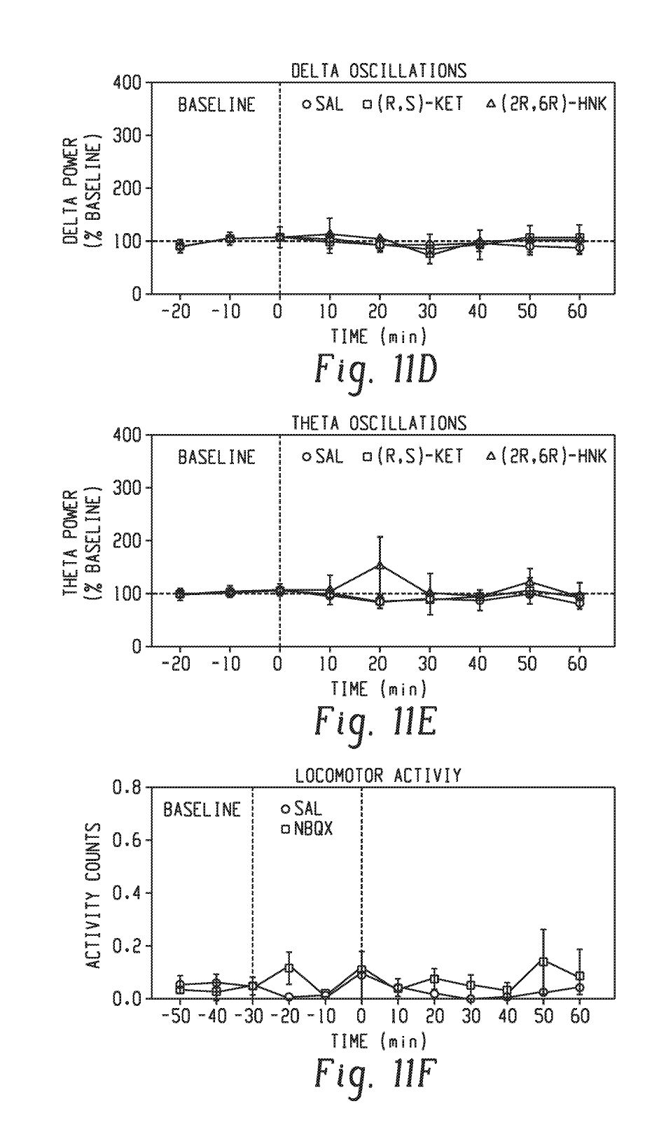

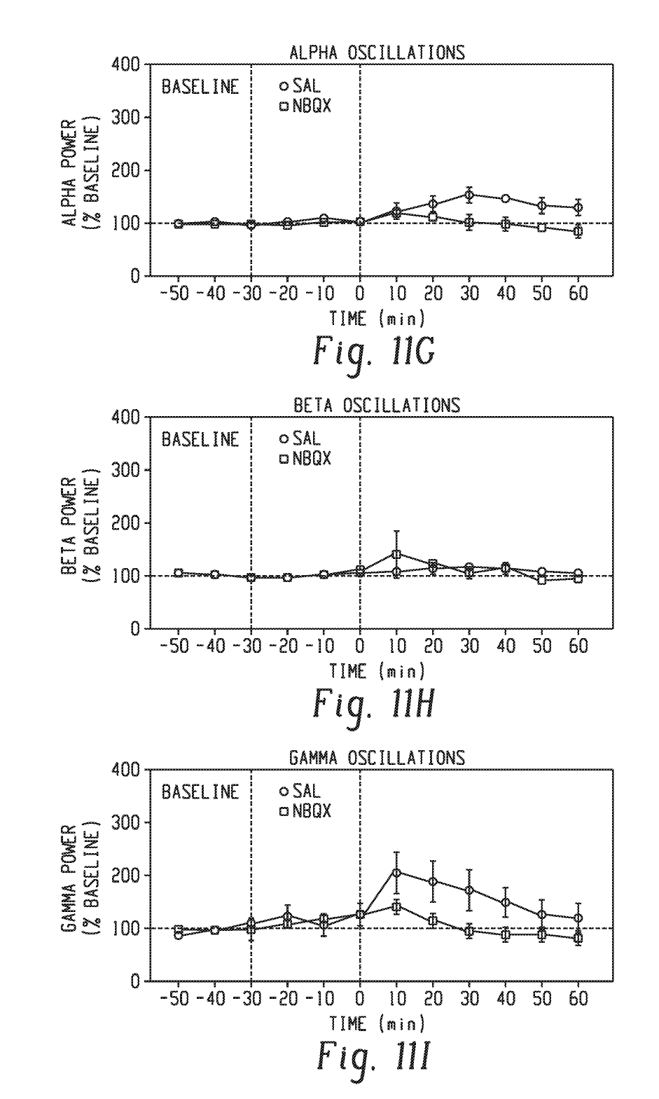

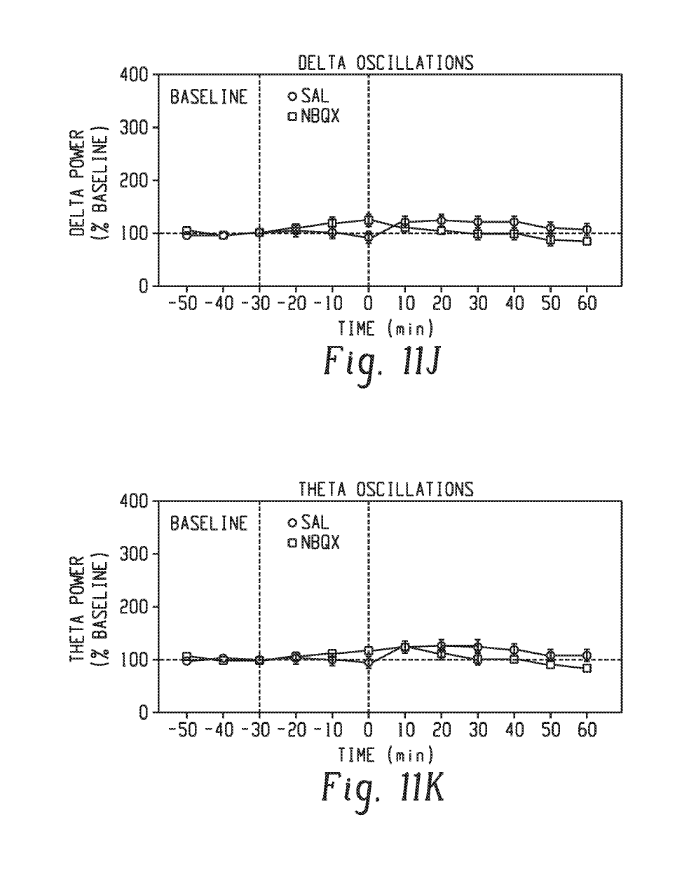

A non-invasive method used to assess ketamine-activated circuitry in both humans and rodents is the quantitative electroencephalography (qEEG) measurement of gamma-band power. This disclosure shows that similar to ketamine, (2R,6R)-HNK administration acutely increases gamma power measured via surface electrodes in vivo (FIG. 3a,3b), independent of locomotor activity changes, and without altering alpha, beta, delta or theta oscillations (FIG. 11a-11e). Gamma power oscillations have been shown to reflect activation of fast ionotropic excitatory receptors, including AMPA receptors. Importantly, we show that pre-administration of the AMPA receptor antagonist 2,3-dihydroxy-6-nitro-7-sulfamoyl-benzo[f]quinoxaline-2,3-dione (NBQX) prevented (2R,6R)-HNK-induced increases in gamma power, thus implicating AMPA receptors in (2R,6R)-HNK mechanism of action (FIG. 11f-11k). To test whether the behavioral antidepressant effects of (2R,6R)-HNK require AMPA receptor activation in vivo, similar to what has been previously shown with ketamine, mice were pretreated with NBQX followed by ketamine or (2R,6R)-HNK (10 min later) and tested 1 hour (FIG. 3c) or 24 hours (FIG. 3d) later in the FST. NBQX pretreatment prevented both the 1- and 24-hour antidepressant effects of (2R,6R)-HNK, indicating that its effects depend on acute activation of AMPA receptors.

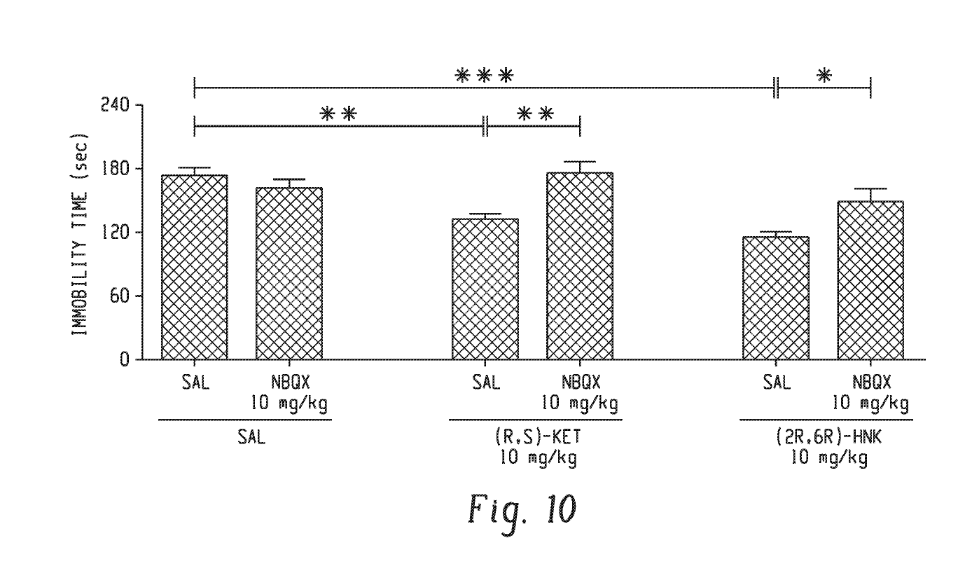

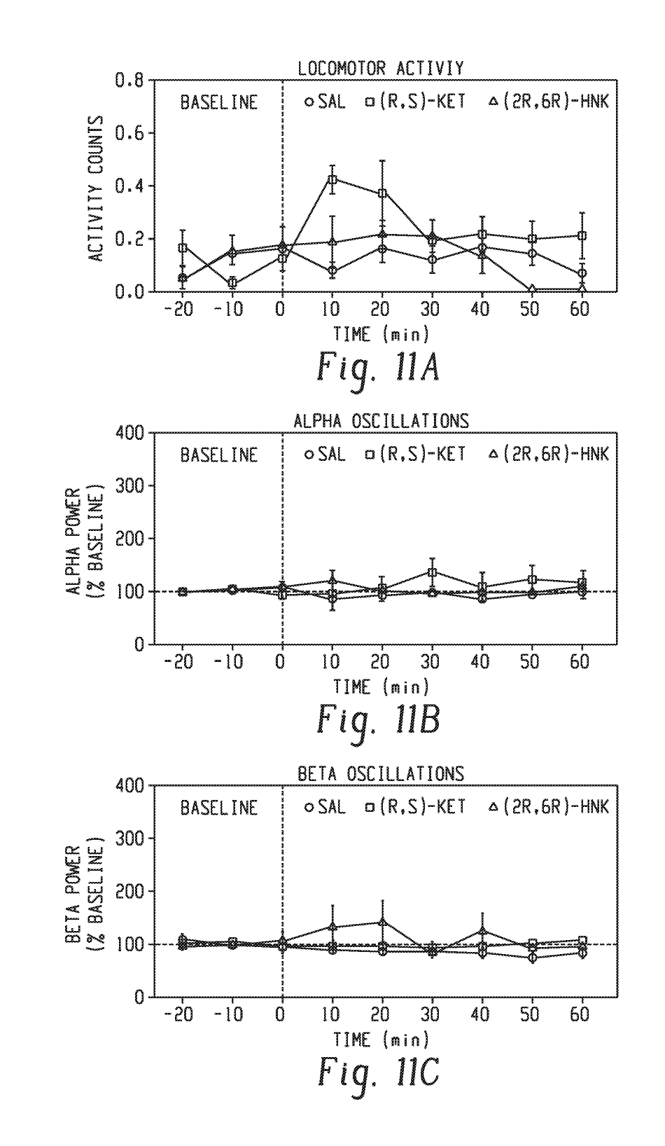

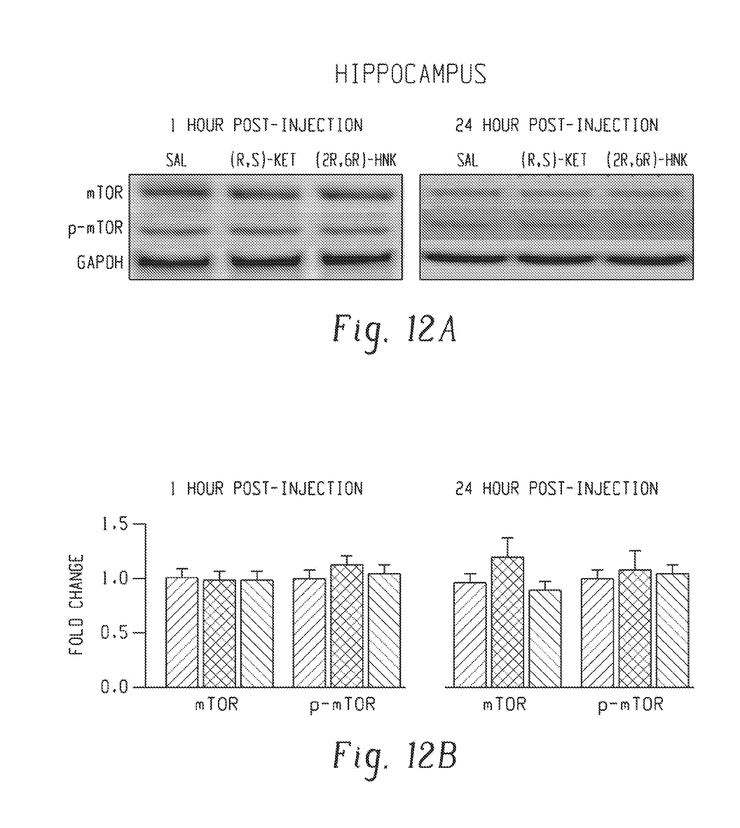

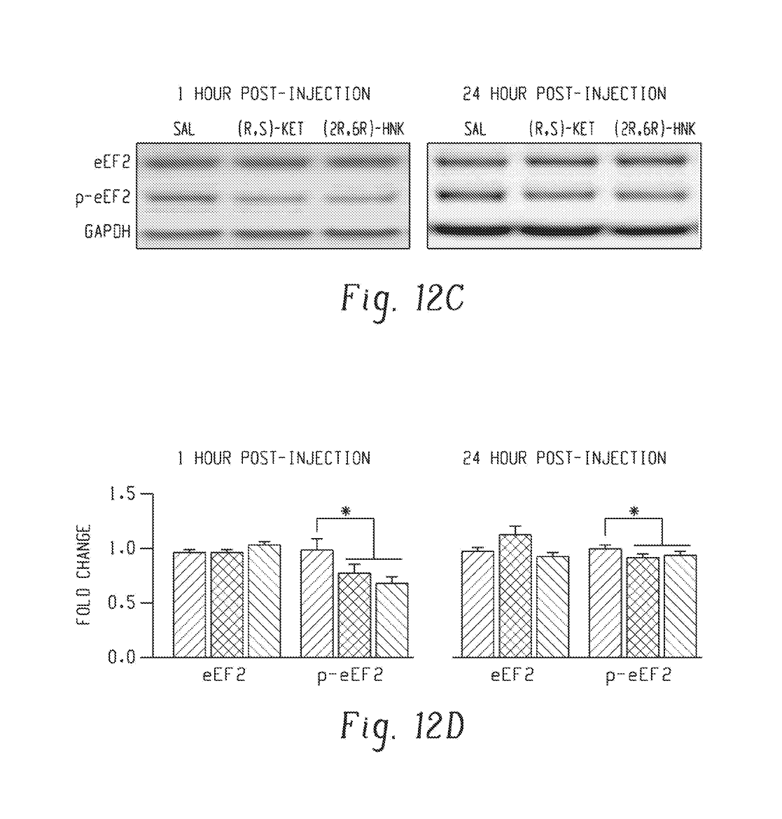

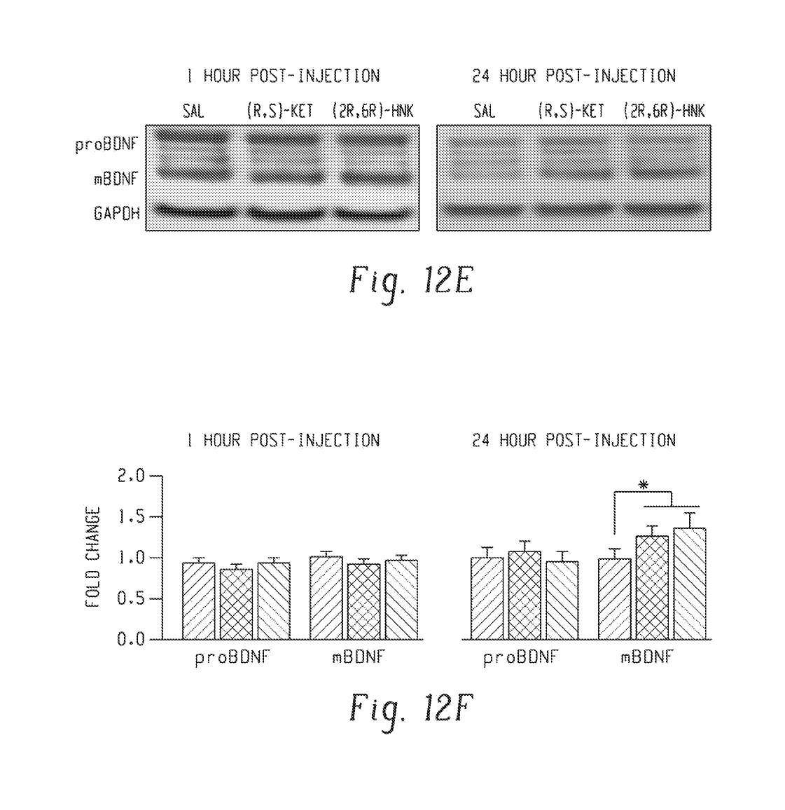

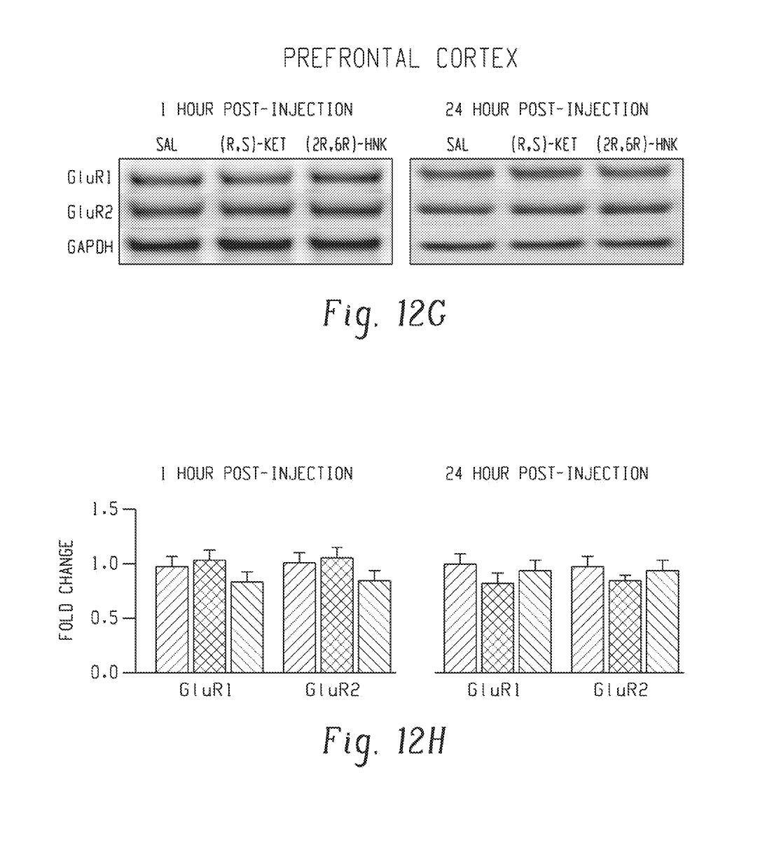

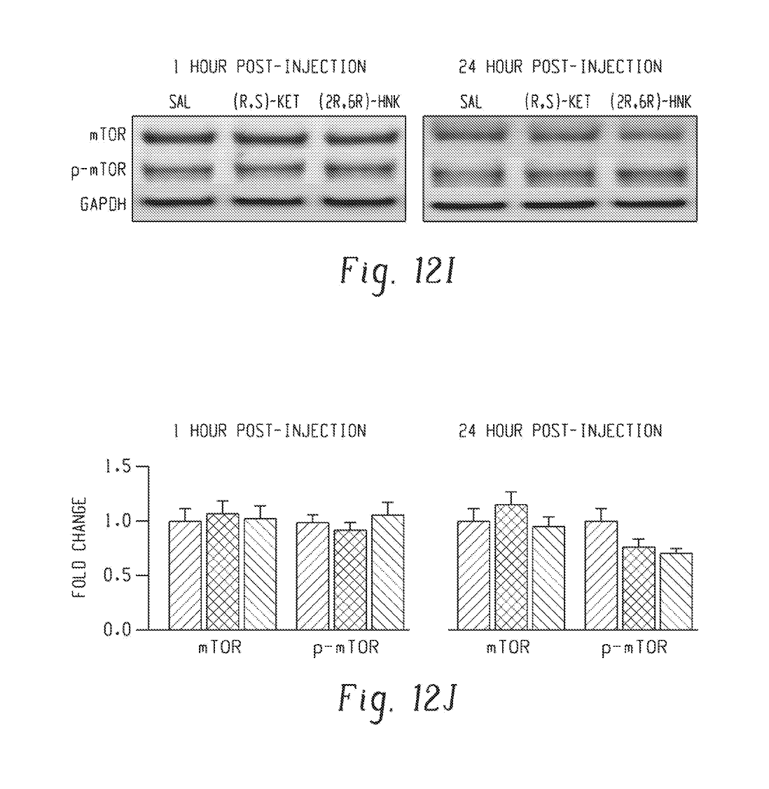

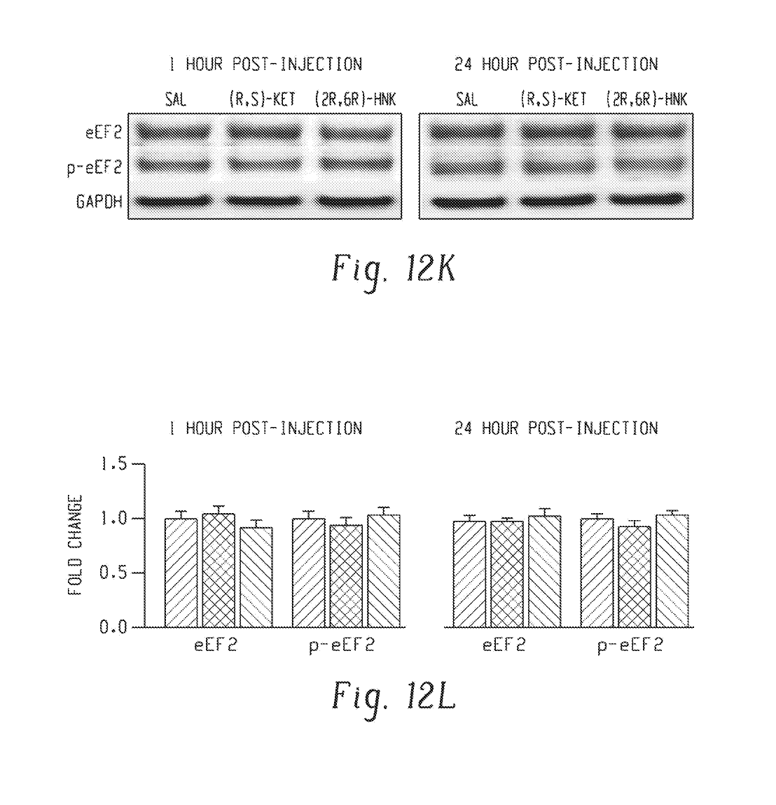

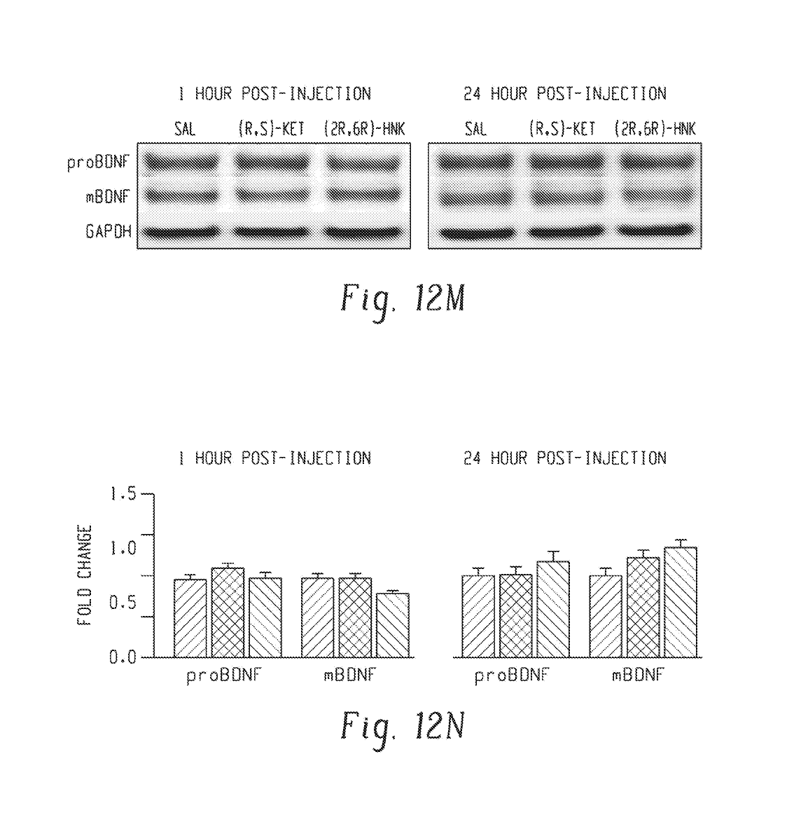

Synaptic plasticity changes involving AMPA receptors have been shown to underlie the long-term antidepressant actions of ketamine. This disclosure shows that while neither ketamine nor (2R,6R)-HNK administration altered the levels of GluR1 and GluR2 in hippocampal synaptoneurosomes 1 hour post-treatment (FIG. 3e), they both increased GluR1 and GluR2 levels 24 hours post-treatment (FIG. 31) in mouse hippocampal, but not prefrontal cortex synaptoneurosomes (FIG. 12g,12h). Consistent with an increase in synaptic AMPA receptors being involved in the sustained, 24-hour, antidepressant actions, administration of NBQX thirty minutes prior to the 24-hour FST (23.5 hours after antidepressant treatment) prevented the antidepressant actions of both ketamine and (2R,6R)-HNK (FIG. 10). Overall, these findings implicate an AMPA receptor activation-dependent initiation and maintenance of synaptic plasticity to underlie the antidepressant effects of (2R,6R)-HNK.

### Example 5. Effects on mTOR, EEF2, and BDNF

Evidence indicates that mTOR signaling, protein synthesis through eEF2 dephosphorylation, as well as BDNF signaling underlie the antidepressant responses of ketamine. Whether administration of (2R,6R)-HNK affects phosphorylation of mTOR (Ser 2448) and eEF2, or BDNF levels in synaptoneurosome fractions of the hippocampus and prefrontal cortex was examined. Regulation of the phosphorylation of mTOR was not observed following administration of ketamine or (2R,6R)-HNK in the hippocampus or the prefrontal cortex of mice (FIG. 12a,12b,12i,12j). However, ketamine induced a decrease in eEF2 phosphorylation in the hippocampus (but not the prefrontal cortex) 1 and 24 hours post-injection, and increased hippocampal BDNF at 24 hours. These effects were recapitulated by (2R,6R)-HNK administration (FIG. 12c,12d,12k,12l,12e,12f,12m,12n).

### Example 6. Effects on Cortical Gamma Power

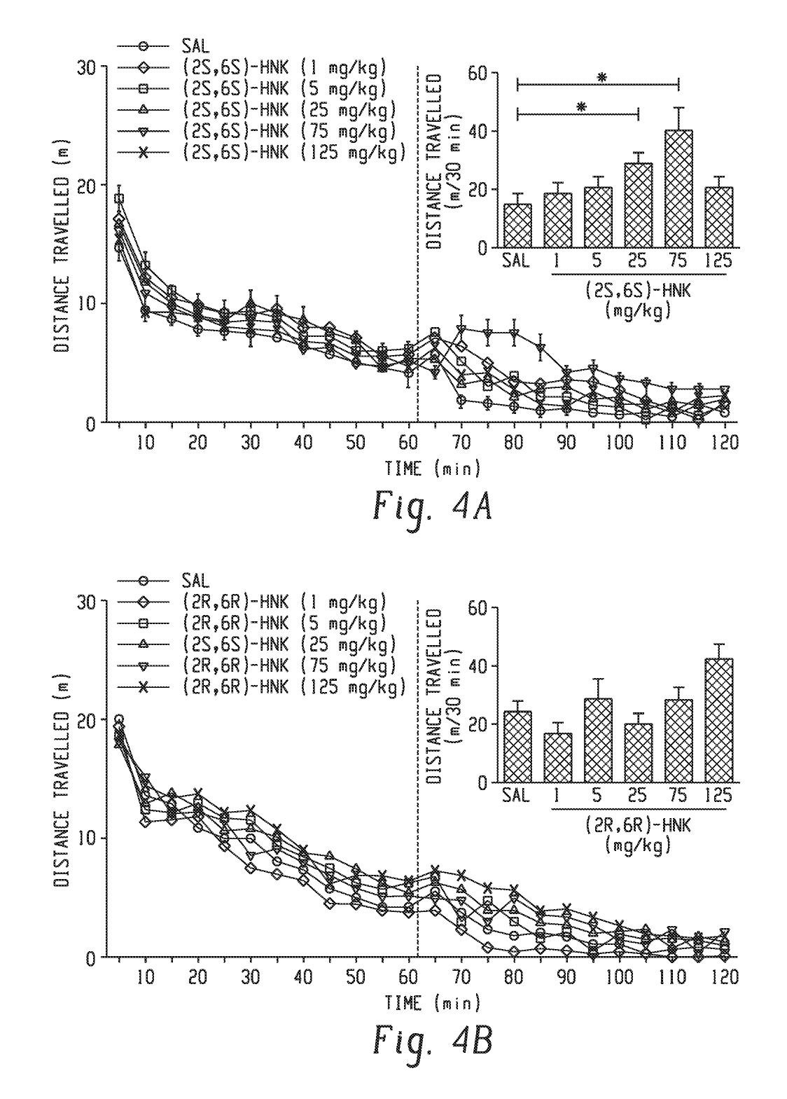

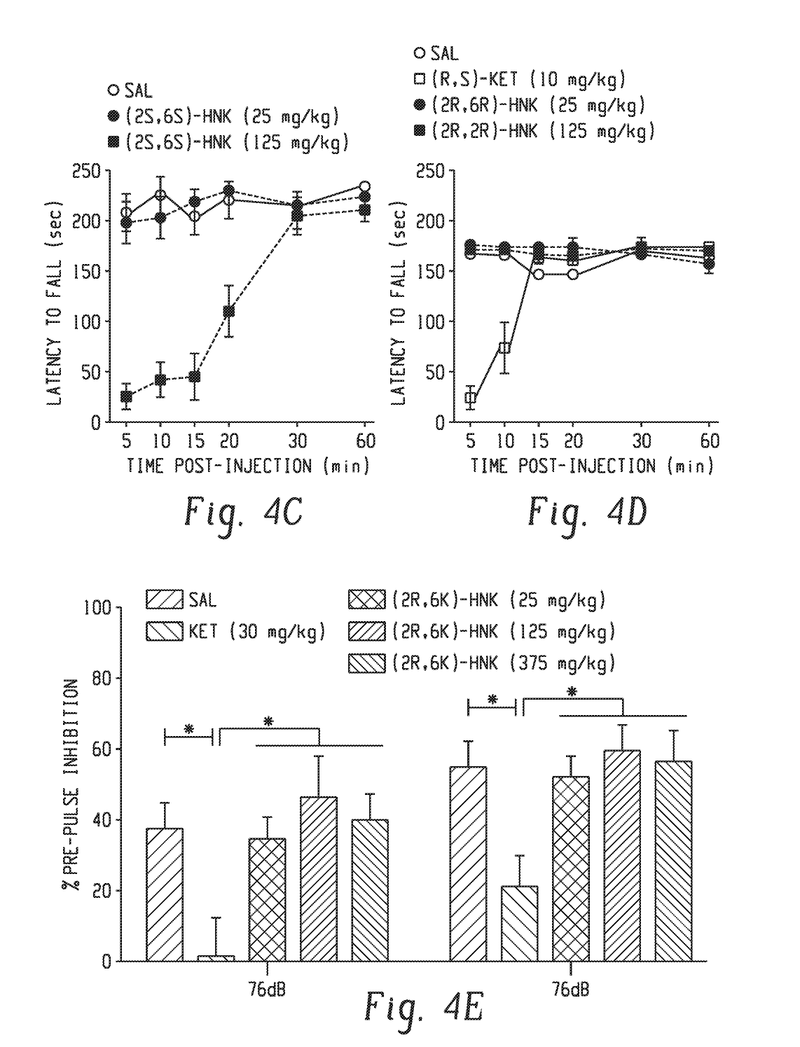

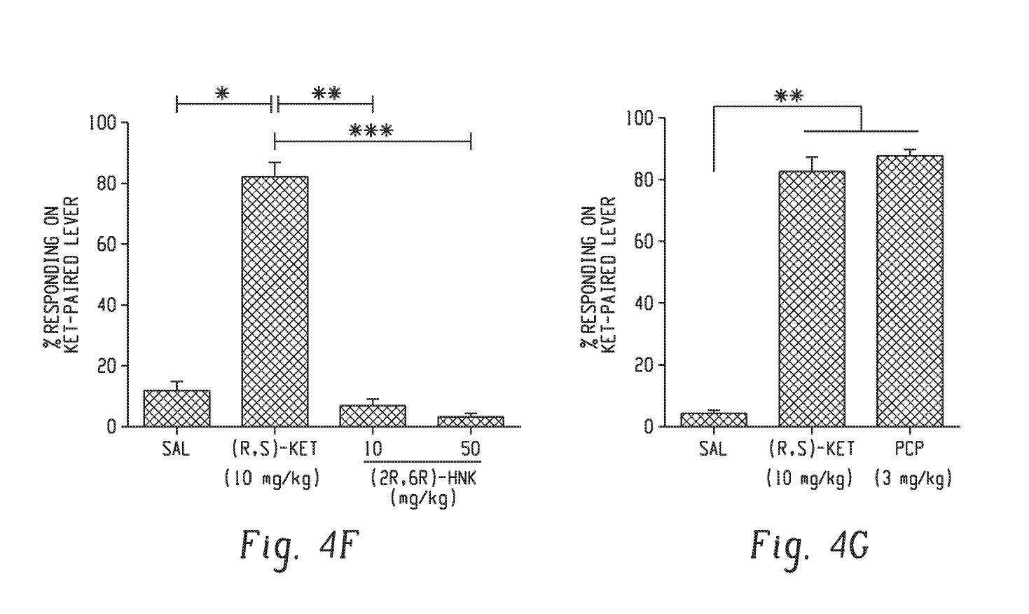

Gamma power oscillations have been hypothesized to reflect activation of fast ionotropic excitatory receptors, including AMPA receptors. A non-invasive method used to assess activation of prefrontal circuits activated by ketamine in both humans and rodents is the quantitative electroencephalography (qEEG) measurement of gamma-band power. Ketamine-induced increases in gamma power are abolished following inhibition of either glutamate release, or AMPA receptors activation, indicating a glutamate- and AMPA-dependent mechanism. Present experiments show that similar to ketamine, (2R,6R)-HNK administration acutely increases cortical gamma power (FIG. 4a-4e), independent of locomotor activity changes induced by ketamine, and without altering alpha, beta, delta or theta oscillations (FIG. 11a-11k).

### Example 7. (2R,6R)-HNK does not Cause Increased Locomotor Activity or Motor Incoordination Compared to Ketamine

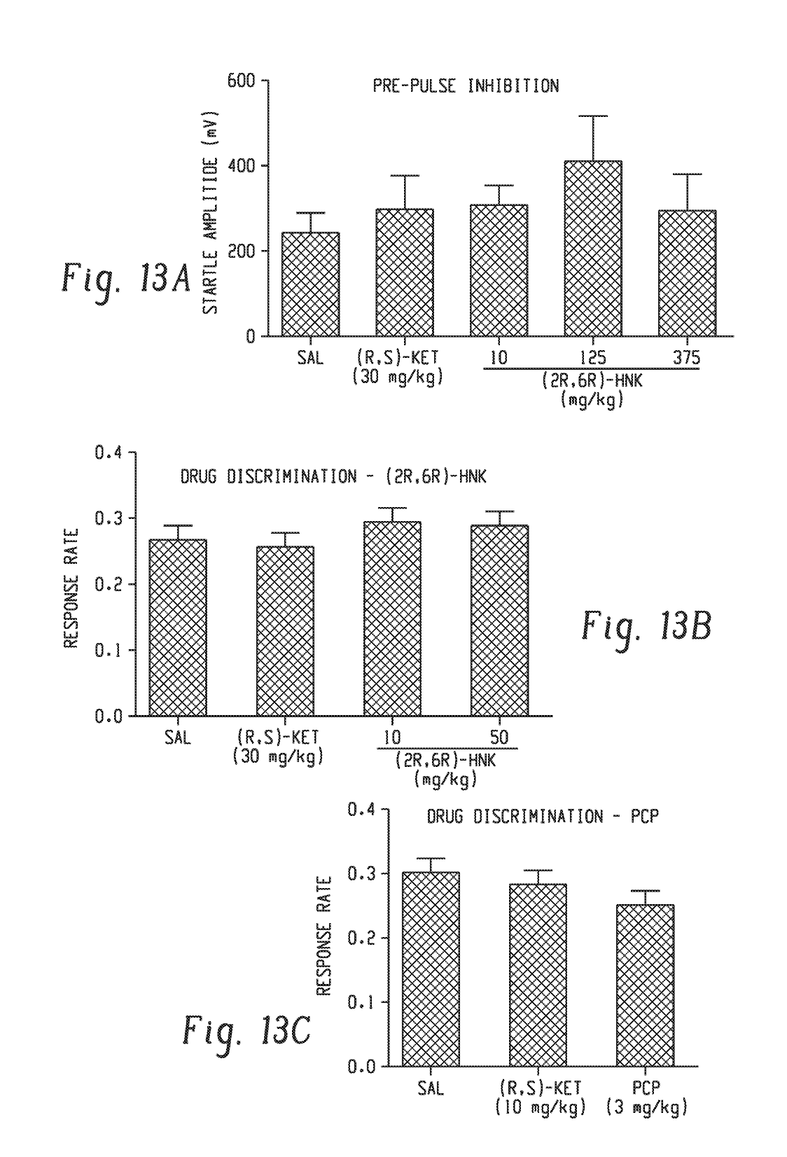

While administration of (2S,6S)-HNK (FIG. 4a) was associated with increased locomotor activity and motor incoordination (FIG. 4c), (2R,6R)-HNK did not induce any significant change in locomotion, and did not affect coordination in the accelerating rotarod test (FIG. 4b,4d). This disclosure shows that (2R,6R)-HNK administration, even at high doses (375 mg/kg), did not affect sensorimotor gating as assessed with pre-pulse inhibition (FIG. 4e) or startle amplitude (FIG. 13a). Non-competitive NMDAR antagonists, including ketamine and phencyclidine, produce discriminative stimulus effects in drug discrimination protocols and manifest cross-drug substitution profiles at an antidepressant-relevant dose range. In ketamine-trained mice, (2R,6R)-HNK administration did not produce ketamine-related discrimination responses, whereas phencyclidine (PCP) did (FIG. 4f,4g; FIG. 13b,13c), further supporting a non-NMDAR mechanism for (2R,6R)-HNK action including interoceptive effects, unlike the abused drugs ketamine and PCP. Overall, (2R,6R)-HNK administration revealed an innocuous side-effect profile compared to ketamine.

### Example 8. Prepulse Inhibition

Experiments were performed to test whether (2R,6R)-HNK inhibits pre-pulse inhibition of the acoustic startle response. Present experiments show that (2R,6R)-HNK administration, even at high doses (375 mg/kg), did not affect pre-pulse inhibition (FIG. 4e) or startle amplitude (FIG. 13a). Non-competitive NMDA receptor antagonists, including ketamine, have been shown to produce discriminative stimulus effects in drug discrimination protocols and have shown cross-drug substitution profiles at an antidepressant-relevant dose range. Here, it is shown that (2R,6R)-HNK administration did not produce discriminative stimulus behaviors, whereas PCP administration produced ketamine-like discriminative properties (FIG. 4g; FIG. 13b, 13c). In addition, (2R,6R)-HNK did not induce any stimulant-like hyperlocomotion, revealing a safe side-effect profile for this metabolite.

### Specific Embodiments

The disclosure includes the following specific embodiments:

### Embodiment 1

A method of treating Psychotic Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, Anhedonia, Post Traumatic Stress Disorder, Unspecified Anxiety Disorder, and fatigue related to mental or medication conditions (e.g, Chronic Fatigue Syndrome, fatigue associated with cancer or other medical conditions or medications to treatment these disorders or conditions), the method comprising administering a pharmaceutical composition containing an effective amount of an active agent, wherein the active agent is purified (2R,6R)-hydroxynorketamine, purified (2S,6S)-hydroxynorketamine, a prodrug thereof, a pharmaceutically acceptable salt of any of the foregoing, or a combination of any of the foregoing.

### Embodiment 2

The method of embodiment 1, wherein the active agent is purified (2R,6R)-hydroxynorketamine or salt thereof.

### Embodiment 3

The method of embodiment 1, wherein the active agent is purified (2S,6S)-hydroxynorketamine or salt thereof.

### Embodiment 4

The method of any of the preceding embodiments, wherein the active agent is administered to the patient together with an additional active agent psychotherapy, talk therapy, cognitive behavioral therapy, exposure therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy, cognitive remediation, exercise, or other types of therapy.

### Embodiment 5

The method of any of the preceding embodiments, wherein the pharmaceutical composition is administered in a dosage form which is an oral, intravenous, intraperitoneal, intranasal subcutaneous, sublingual, intrathecal, transdermal, buccal, vaginal, or rectal dosage form.

### Embodiment 6

The method of any of the preceding embodiments, wherein the unitdosage form contains an amount of the active agent of from 1 mg to 5000 mg, from 1 mg to 2000 mg, from 1 mg to 1000 mg, from 1 mg to 500 mg, from 1 mg to 50 mg, from 10 mg to 200 mg, from 10 mg to 500 mg, or from 10 mg to 200 mg.

### Embodiment 7

The method of embodiments 1 to 5 wherein 0.005 mg/kg to 50 mg/kg, 0.01 mg/kg to 10 mg/kg, 0.05 mg/kg to 10 mg/kg, or 0.1 mg/kg to 5 mg/kg of the active agent is administered to the patient in a 24 hour period.

### Embodiment 8

The method according of any of the preceding embodiments, wherein the dosage form is administered to the patient once per day, twice per day, three times per day, or four times per day.

### Embodiment 9

The method of any of the preceding embodiments, wherein the dosage form is administered to the patient as an infusion over a period of 10 minutes to 24 hours, 30 minutes to 12 hours, 10 minutes to 10 hours, 10 minutes to 4 hours, or 30 minutes to 4 hours.

### Embodiment 10

The method of any of the preceding embodiments of treating Psychotic Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, where an effective amount of the compound is an amount effective to decrease depressive symptoms, wherein a decrease in depressive symptoms is the achievement of
a 50% or greater reduction of symptoms identified on a depression symptom rating scale, or
a score less than or equal to 7 on the HRSD₁₇, or
less than or equal to 5 on the QID-SR₁₆, or
less than or equal to 10 on the MADRS.

### Embodiment 11

The method of any one of embodiments 1 to 9 for treating fatigue, where an effective amount of the compound is an amount effective to decrease fatigue symptoms, wherein a decrease in fatigue symptoms is the achievement of a 50% or greater reduction of fatigue symptoms identified on a fatigue symptom rating scale.

### Embodiment 12

The method of any of embodiments 1 to 9 of treating Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, and Unspecified Anxiety Disorder, wherein an effective amount is an amount effective to decrease anxiety symptoms; wherein a decrease in anxiety symptoms is the achievement of
a 50% or greater reduction of anxiety symptoms on an anxiety symptom rating scale, or
a score less than or equal to 39 on the STAI, or
less than or equal to 9 on the BAI, or
less than or equal to 7 on the HADS-A.

### Embodiment 13

The method of any one of embodiments 1-8 of treating Anhedonia, wherein an effective amount is an amount effective to decrease Anhedonia, wherein a decrease in Anhedonia is the achievement of a clinically significant decrease in Anhedonia on an Anhedonia rating scale, wherein the Anhedonia rating scale is the Shaith-Hamilton Pleasure Scale (SHAPS and SHAPS-C) or the Temporal Experience of Pleasure Scale (TEPS).

### Embodiment 14

The method of any one of embodiments 1-9 of treating suicidal ideation, wherein an effective amount is an amount effective to decrease suicidal ideation, wherein a decrease in suicide ideation is the achievement of a clinically significant decrease in suicidal ideation on a suicidal ideation rating scale, wherein the suicidal ideation rating scale is Scale for Suicidal Ideation (SSI), the Suicide Status Form (SSF), or the Columbia Suicide Severity Rating Scale (C-SSRS).

### Embodiment 15

The method of any of the preceding embodiments, wherein the patient is human. In certain embodiments the patient may be a non-human animal such as a livestock animal or a companion animal such as a cat or dog.

### Embodiment 16

The method of any one of the preceding claims, additionally comprising determining whether the patient is a ketamine non-responder or a ketamine responder and administering an efficacious amount of active agent based on the patient's status as a ketamine non-responder or ketamine responder. Additional embodiments include the method of any of the preceding claims in which any one of the disorders listed in claim 1 is the only disorder listed in the embodiment.

1. A method of treating Psychotic Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, Anhedonia, Post Traumatic Stress Disorder, Unspecified Anxiety Disorder, or fatigue the method comprising administering a pharmaceutical composition containing an effective amount of an active agent, wherein the active agent is purified (2R,6R)-hydroxynorketamine, purified (2S,6S)-hydroxynorketamine, a prodrug thereof, a pharmaceutically acceptable salt of any of the foregoing thereof or a combination thereof, together with a pharmaceutically acceptable carrier to a patient in need of such treatment.

2. The method of claim 1, wherein the active agent is purified (2R,6R)-hydroxynorketamine or salt thereof.

3. The method of claim 1, wherein the active agent is purified (2S,6S)-hydroxynorketamine or salt thereof.

4. The method of claim 1, wherein the active agent is administered to the patient together with an additional active agent or administered together with psychotherapy, talk therapy, cognitive behavioral therapy, exposure therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy, cognitive remediation, exercise, or other types of therapy.

5. The method of claim 1, wherein the pharmaceutical composition is administered in a dosage form which is an oral, intravenous, intraperitoneal, intranasal, subcutaneous, sublingual, intrathecal, transdermal, buccal, vaginal, or rectal dosage form.

6. The method according to claim 5, wherein the unit dosage of the dosage form contains an amount of the active agent of from 1 mg to 5000 mg, from 1 mg to 1000 mg, from 1 mg to 500 mg, or from 10 mg to 200 mg.

7. The method according to claim 5, wherein 0.005 mg/kg to 50 mg/kg, 0.05 mg/kg to 10 mg/kg, or 0.1 mg/kg to 5 mg/kg of the active agent is administered to the patient in a 24 hour period.

8. The method of claim 5, wherein the dosage form is administered to the patient once per day, twice per day, three times per day, or four times per day.

9. The method according to claim 5, wherein the dosage form is administered to the patient as an infusion over a period of 10 minutes to 24 hours, or 30 minutes to 12 hours, or 30 minutes to 4 hours.

10. The method of claim 1 of treating Psychotic Depression, Suicidal Ideation, Disruptive Mood Dysregulation Disorder, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, or fatigue where an effective amount of the compound is an amount effective to decrease depressive symptoms, wherein a decrease in depressive symptoms is the achievement of
a 50% or greater reduction of symptoms identified on a depression symptom rating scale, or
a score less than or equal to 7 on the HRSD₁₇, or
less than or equal to 5 on the QID-SR₁₆, or
less than or equal to 10 on the MADRS.

11. A method for treating fatigue, where an effective amount of the compound is an amount effective to decrease fatigue symptoms, wherein a decrease in fatigue symptoms is the achievement of a 50% or greater reduction of fatigue symptoms identified on a fatigue symptom rating scale, the method comprising administering a pharmaceutical composition containing an effective amount of an active agent, wherein the active agent is purified (2R,6R)-hydroxynorketamine, purified (2S,6S)-hydroxynorketamine, a prodrug thereof, a pharmaceutically acceptable salt of any of the foregoing thereof or a combination thereof, together with a pharmaceutically acceptable carrier to a patient in need of such treatment.

12. The method of claim 1 of treating Separation Anxiety Disorder, Selective Mutism, Specific Phobia, Social Anxiety Disorder (Social Phobia), Panic Disorder, Panic Attack (Specifier), Agoraphobia, Generalized Anxiety Disorder, Substance/Medication-Induced Anxiety Disorder, Anxiety Disorder Due to Another Medical, Other Specified Anxiety Disorder, and Unspecified Anxiety Disorder, wherein an effective amount is an amount effective to decrease anxiety symptoms; wherein a decrease in anxiety symptoms is the achievement of
a 50% or greater reduction of anxiety symptoms on an anxiety symptom rating scale, or
a score less than or equal to 39 on the STAT, or
less than or equal to 9 on the BAT, or
less than or equal to 7 on the HADS-A.

13. A method of treating Anhedonia, wherein an effective amount is an amount effective to decrease Anhedonia, wherein a decrease in Anhedonia is the achievement of a clinically significant decrease in Anhedonia on an Anhedonia rating scale, wherein the Anhedonia rating scale is the Shaith-Hamilton Pleasure Scale (SHAPS and SHAPS-C) or the Temporal Experience of Pleasure Scale (TEPS), the method comprising administering a pharmaceutical composition containing an effective amount of an active agent, wherein the active agent is purified (2R,6R)-hydroxynorketamine, purified (2S,6S)-hydroxynorketamine, a prodrug thereof, a pharmaceutically acceptable salt of any of the foregoing thereof or a combination thereof, together with a pharmaceutically acceptable carrier to a patient in need of such treatment.

14. The method of claim 1 of treating suicidal ideation, wherein an effective amount is an amount effective to decrease suicidal ideation, wherein a decrease in suicidal ideation is the achievement of a clinically significant decrease in suicidal ideation on a suicidal ideation rating scale, wherein the suicidal ideation rating scale is Scale for Suicidal Ideation (SSI), the Suicide Status Form (SSF), or the Columbia Suicide Severity Rating Scale (C-SSRS).

15. The method of any of claims 1 to 13 wherein the patient is human.

16. The method of claim 1, additionally comprising determining whether the patient is a ketamine non-responder or a ketamine responder and administering an efficacious amount of active agent based on the patient's status as a ketamine non-responder or ketamine responder.

17. The method of claim 11, wherein the active agent is purified (2R,6R)-hydroxynorketamine or salt thereof.

18. The method of claim 17, additionally comprising determining whether the patient is a ketamine non-responder or a ketamine responder and administering an efficacious amount of active agent based on the patient's status as a ketamine non-responder or ketamine responder.

19. The method of claim 13, wherein the active agent is purified (2R,6R)-hydroxynorketamine or salt thereof.

20. The method of claim 17, additionally comprising determining whether the patient is a ketamine non-responder or a ketamine responder and administering an efficacious amount of active agent based on the patient's status as a ketamine non-responder or ketamine responder.
